(19) United States
(12) Patent Application Publication
(10) Pub. No.: US 2025/0215091 A1
(43) Pub. Date: Jul. 3, 2025

(54) Anti-GAL3 Antibodies and Compositions

(71) Applicant: Les Laboratoires Servier, Suresnes Cedex (FR)

(72) Inventors: Frédéric DE CEUNINCK, Paris (FR); Céline ORTEIGA-FERREIRA, Longpont sur Orge (FR); Catherine GALLOU, Gômetz Le Chatel (FR); Gautier ROBIN, Montpellier (FR)

(73) Assignee: Les Laboratoires Servier, Suresnes Cedex (FR)

(21) Appl. No.: 18/850,892

(22) PCT Filed: Mar. 24, 2023
(86) PCT No.: PCT/EP2023/057647

§ 371 Date: Sep. 25, 2024

(30) Foreign Application Priority Data
Mar. 25, 2022 (EP) 22305372.9
Aug. 4, 2022 (EP) 22306186.2

(51) Int. Cl. C07K 16/28; A61K 39/00; A61P 11/00; A61P 17/00

(52) U.S. Cl. CPC C07K 16/2851; A61P 11/00; A61P 17/00; A61K 2039/505; C07K 2317/33; C07K 2317/622; C07K 2317/76; C07K 2317/92

(57) ABSTRACT

This invention relates to anti-Gal3 antibodies and methods of using them in treating diseases and conditions that benefit from modulating Gal3 activity, e.g., fibrotic disease, inflammatory disease, autoimmune disease, 5 immune-mediated disorder, cancer, neurodegenerative disease, metabolic disease or infectious disease.

### BACKGROUND OF THE INVENTION

Galectins are multifunctional proteins that specifically bind to beta-galactoside containing ligands through their carbohydrate recognition domain (CRD).

Galectin-3 (Gal3) is a member of this family encompassing 15 galectins in mammals, which are evolutionary highly conserved. Most galectins contain two carbohydrate recognition domain (CRD), that are either homodimers (Galectin-1, -2, -5, -7, -10, -11, -13, 14, -15) or heterodimers (Galectin-5, -6, -8, -9, -12). Gal3 is the only monomeric galectin, comprising a single CRD linked to a collagen-like N-terminal domain, and is also described to undergo oligomerisation through this N-terminal domain depending on environmental conditions.

Gal3 is able to bind several carbohydrate-containing ligands with preferential recognition for N-acetyl-lactosamine structures present in intracellular, membrane-associated or extracellular proteins. Asialofetuin (ASF) glycoprotein is one of the prototypical ligand of Gal3, and is also able to associate with Galectin-1 (Gall) or Galectin-7 (Gal7).

Gal3 is a multicompartment protein, present intracellularly, extracellularly, or associated with cell membranes. Intracellularly, Gal3 has been described to regulate several processes linked to cell growth or apoptotic pathways, or cell signalling events. When associated to ligands of the plasma membrane, Gal3 has been observed to influence the expression, localization, and activity of several cell surface receptors, thus modulating various biological functions such as cell migration or cell adhesion. In the extracellular compartment Gal3 is involved mainly in maintaining the integrity of the extracellular matrix and cell adhesion processes. In some conditions, disruption of the expression of Gal3 and/or its extracellular SUBSTITUTE SHEET (RULE 26) interactome has been shown to trigger several pathological processes linked to inflammation, fibrosis dysregulation of the immune system, angiogenesis, tumour metastasis and progression, and cellular homeostasis.

Gal3 is widely expressed in cells of the innate immune system among others and has been shown to stimulate inflammatory responses by increasing the chemoattraction and stimulating the production of proinflammatory cytokines by cells of the myeloid lineage, including macrophages. Gal3 is also expressed by fibroblasts and endothelial cells and, in combination with activated macrophages, promotes tissue fibrosis through proliferation of fibroblasts and increased collagen production.

Increased Gal3 expression is associated with a number of fibrotic diseases including heart failure, kidney failure, systemic sclerosis, lung fibrosis, hepatic fibrosis, atherosclerosis, as well as inflammatory diseases, autoimmune diseases, immune-mediated disorders, neurodegenerative diseases, metabolic diseases, infectious diseases and different kind of cancers. Inhibition of Gal3 interaction is therefore an attractive mechanism in the treatment of cited conditions.

Primary modalities used for targeting Gal3 have been small molecules and large complex carbohydrates such as pectin-derivatives. They are often described as being able to target Gal3, but sometimes other galectins, thus lacking selectivity

For instance, TD139 (GB-0139) developed by Galecto Inc. and Bristol-Myers Squibb or Belapectin (GR-MD-02) developed by Galectin Therapeutics are said to be dual inhibitors of Gal3 and Gall. They thus respectively show poor selectivity for Gal3. Besides, TD139 has poor bioavailability and is formulated for inhalation twice a day being able to reach primarily the lung and developed for the treatment of idiopathic pulmonary fibrosis. Such administration route could also be a disadvantage for the treatment of other conditions.

This dual selectivity is also associated with the complex carbohydrates Davanat (GM-CT-01, also developed by Galectin Therapeutics) or GCS-100 developed by La Jolla Pharmaceuticals.

Another small molecule in development is GB1211 from Galecto Inc. which is an oral inhibitor and is described as targeting Gal3 including intracellular Gal3, due to its ability to cross the cell plasma membrane, distinct from our antibodies targeting extracellular Gal3.

The identification of small molecules or complex carbohydrates Gal3 inhibitors have encountered many hurdles resulting from complex and difficult glycochemistry design and potentially leading to compounds with low selectivity and/or potency and/or bioavailability.

Therapeutic approach with antibodies is suited to reach a target with low druggability and could show higher selectivity, potency and bioavailability compared to chemical entities and pectin derivatives.

An anti-Gal3 antibody has recently been described by True Binding, whose TB001 monoclonal antibody is in preclinical phase and TB006 monoclonal antibody is in phase 1 in the indications of Alzheimer's disease and acute ischemic stroke. These antibodies are described as inhibitors of Gal3 and TIM-3 interaction. They are not described as targeting specifically Gal3-CRD.

Thus there is a need to develop new therapeutic entities targeting specifically Gal3, particularly Gal3 in its CRD, to reach the site of interaction with its ligands, avoiding selectivity, bioavailability or potency concerns encountered with small molecule compounds or pectin derivatives in development.

### SUMMARY OF THE INVENTION

The present invention is directed to novel monoclonal antibodies binding to extracellular Galectin-3 (Gal3), particularly binding to carbohydrate recognition domain of Gal3 (Gal3-CRD), and binding neither to Galectin-1 (Gall) nor to Galectin-7 (Gal7).

The present invention is also directed to pharmaceutical compositions comprising one or more of these antibodies, and use of the antibodies and pharmaceutical compositions for inhibition of disease progression in a patient or for amelioration, prevention, and/or treatment of a patient suffering from Gal3-related disease or Gal3-related disorder, including fibrotic diseases, such as heart failure, kidney failure, systemic sclerosis, lung fibrosis and hepatic fibrosis, and including inflammatory diseases, autoimmune diseases, neurodegenerative diseases, metabolic diseases, infectious diseases as well as several cancers. Compared to currently available molecules in development targeting Gal3-related disease or Gal3-related disorder, it is contemplated that the antibodies of the present invention show specific properties either alone or in combination with another therapeutic molecule.

In one embodiment, the present invention provides an anti-Gal3 antibody or an antigen-binding fragment thereof, wherein the anti-Gal3 antibody or antigen-binding fragment thereof is any of the antibodies referred to herein as antibody D06, D11, E01, E02, E07, G03, H07, H10, E12 or B12, or a variant thereof, where the variant may contain certain minimum amino acid changes relative to antibody D06, D11, E01, E02, E07, G03, H07, H10, E12 or B12, without losing the antigen-binding specificity of the parent antibody. In one embodiment, the anti-Gal3 antibody or antigen-binding fragment thereof competes for binding to human Gal3 with an antibody whose heavy chain (H) CDR1-3 and light chain (L) CDR1-3 are the same as or derived from the H-CDR1-3 and L-CDR1-3 of antibody D06, D11, E01, E02, E07, G03, H07, H10, E12 or B12.

In one embodiment, the anti-Gal3 antibody or antigen-binding fragment thereof comprises H-CDR1-3 comprising the H-CDR1-3 sequences, respectively, of antibody D06, D11, E01, E02, E07, G03, H07, H10, E12 or B12.

In one embodiment, the anti-Gal3 antibody or antigen-binding fragment thereof comprises L-CDR1-3 comprising the L-CDR1-3 sequences, respectively, of antibody D06, D11, E01, E02, E07, G03, H07, H10, E12 or B12.

In one embodiment, the anti-Gal3 antibody or antigen-binding fragment thereof comprises the H-CDR1-3 and L-CDR1-3 amino acid sequences of antibody D06, D11, E01, E02, E07, G03, H07, H10, E12 or B12.

In one embodiment, the anti-Gal3 antibody or antigen-binding fragment thereof comprises a heavy chain complementarity-determining region (H-CDR) 1 comprising the amino acid sequence of any one of SEQ ID NO: 1-5.

In one embodiment, the anti-Gal3 antibody or antigen-binding fragment thereof comprises a heavy chain complementarity-determining region (H-CDR) 2 comprising the amino acid sequence of any one of SEQ ID NO: 6-11.

In one embodiment, the anti-Gal3 antibody or antigen-binding fragment thereof comprises a heavy chain complementarity-determining region (H-CDR) 3 comprising the amino acid sequence of any one of SEQ ID NO: 12-19.

In one embodiment, the anti-Gal3 antibody or antigen-binding fragment thereof comprises a heavy chain complementarity-determining region (L-CDR) 1 comprising the amino acid sequence of any one of SEQ ID NO: 20-26.

In one embodiment, the anti-Gal3 antibody or antigen-binding fragment thereof comprises a heavy chain complementarity-determining region (L-CDR) 2 comprising the amino acid sequence of any one of SEQ ID NO: 27-29.

In one embodiment, the anti-Gal3 antibody or antigen-binding fragment thereof comprises a heavy chain complementarity-determining region (L-CDR) 3 comprising the amino acid sequence of any one of SEQ ID NO: 30-37.

In one embodiment, the anti-Gal3 antibody or antigen-binding fragment thereof has a heavy chain variable domain (VH) that is at least 90% (e.g., at least 92%, at least 95%, at least 98%, or at least 99%) identical in sequence to the VH amino acid sequence of SEQ ID NO: 38, 40, 42, 44, 46, 48, 50, 52, 54, 56, 81, 82, 84, 85, 87, 88, 90, 92, 94, 95, 97, 99, 101, 103 or 104.

In one embodiment, the anti-Gal3 antibody or antigen-binding fragment thereof has a VH that comprises SEQ ID NO: 38, 40, 42, 44, 46, 48, 50, 52, 54, 56, 81, 82, 84, 85, 87, 88, 90, 92, 94, 95, 97, 99, 101, 103 or 104.

In one embodiment, the anti-Gal3 antibody or antigen-binding fragment thereof has a heavy chain (HC) that comprises the VH amino acid sequence of SEQ ID NO: 38, 40, 42, 44, 46, 48, 50, 52, 54, 56, 81, 82, 84, 85, 87, 88, 90, 92, 94, 95, 97, 99, 101, 103 or 104 and the heavy chain constant region amino acid sequence of SEQ ID NO: 58 or 59.

In one embodiment, the anti-Gal3 antibody or antigen-binding fragment thereof has a light chain variable domain (VL) that is at least 90% (e.g., at least 92%, at least 95%, at least 98%, or at least 99%) identical in sequence to the VL amino acid sequence of SEQ ID NO: 39, 41, 43, 45, 47, 49, 51, 53, 55, 57, 83, 86, 89, 91, 93, 96, 98, 100, 102 or 105.

In one embodiment, the anti-Gal3 antibody or antigen-binding fragment thereof has a VL that comprises SEQ ID NO: 39, 41, 43, 45, 47, 49, 51, 53, 55, 57, 83, 86, 89, 91, 93, 96, 98, 100, 102 or 105.

In one embodiment, the anti-Gal3 antibody or antigen-binding fragment thereof has a light chain (LC) that comprises the VL amino acid sequence of SEQ ID NO: 39, 41, 43, 45, 47, 49, 51, 53, 55, 57, 83, 86, 89, 91, 93, 96, 98, 100, 102 or 105 and the light chain constant region amino acid sequence of SEQ ID NO: 60.

In certain embodiments, the anti-Gal3 antibody or antigen-binding fragment thereof of the invention comprises the H-CDR 1-3 and L-CDR 1-3 amino acid sequences of:
a) SEQ ID NOs: 1, 6, 12, 20, 27 and 30, respectively;
b) SEQ ID NOs: 1, 6, 13, 21, 27 and 31, respectively;
c) SEQ ID NOs: 1, 6, 14, 22, 27 and 31, respectively;
d) SEQ ID NOs: 5, 7, 3, 21, 28 and 32, respectively;
e) SEQ ID NOs: 3, 8, 15, 23, 27 and 33, respectively;
f) SEQ ID NOs: 2, 6, 16, 24, 27 and 34, respectively;
g) SEQ ID NOs: 1, 9, 14, 24, 27 and 34, respectively;
h) SEQ ID NOs: 2, 10, 17, 25, 27 and 35, respectively;
i) SEQ ID NOs: 4, 11, 18, 21, 29 and 36, respectively; or
j) SEQ ID NOs: 2, 10, 19, 26, 27 and 37, respectively.

In certain embodiments, the anti-Gal3 antibody or antigen-binding fragment thereof of the invention:
a) has a VH that is at least 90% (e.g, at least 92%, at least 95%, at least 98%, or at least 99%) identical in sequence to the amino acid sequence of SEQ ID NO: 38, 81 or 82, and a VL that is at least 90% (e.g, at least 92%, at least 95%, at least 98%, or at least 99%) identical in sequence to the amino acid sequence of SEQ ID NO: 39 or 83;
b) has a VH that is at least 90% (e.g, at least 92%, at least 95%, at least 98%, or at least 99%) identical in sequence to the amino acid sequence of SEQ ID NO: 40, 84 or 85, and a VL that is at least 90% (e.g, at least 92%, at least 95%, at least 98%, or at least 99%) identical in sequence to the amino acid sequence of SEQ ID NO: 41 or 86;
c) has a VH that is at least 90% (e.g, at least 92%, at least 95%, at least 98%, or at least 99%) identical in sequence to the amino acid sequence of SEQ ID NO: 42, 87 or 88, and a VL that is at least 90% (e.g, at least 92%, at least 95%, at least 98%, or at least 99%) identical in sequence to the amino acid sequence of SEQ ID NO: 43 or 89;
d) has a VH that is at least 90% (e.g, at least 92%, at least 95%, at least 98%, or at least 99%) identical in sequence to the amino acid sequence of SEQ ID NO: 44 or 90, and a VL that is at least 90% (e.g, at least 92%, at least 95%, at least 98%, or at least 99%) identical in sequence to the amino acid sequence of SEQ ID NO: 45 or 91;
e) has a VH that is at least 90% (e.g, at least 92%, at least 95%, at least 98%, or at least 99%) identical in sequence to the amino acid sequence of SEQ ID NO: 46 or 92, and a VL that is at least 90% (e.g, at least 92%, at least 95%, at least 98%, or at least 99%) identical in sequence to the amino acid sequence of SEQ ID NO: 47 or 93;
f) has a VH that is at least 90% (e.g, at least 92%, at least 95%, at least 98%, or at least 99%) identical in sequence to the amino acid sequence of SEQ ID NO: 48, 94 or 95, and a VL that is at least 90% (e.g, at least 92%, at least 95%, at least 98%, or at least 99%) identical in sequence to the amino acid sequence of SEQ ID NO: 49 or 96;
g) has a VH that is at least 90% (e.g, at least 92%, at least 95%, at least 98%, or at least 99%) identical in sequence to the amino acid sequence of SEQ ID NO: 50 or 97, and a VL that is at least 90% (e.g, at least 92%, at least 95%, at least 98%, or at least 99%) identical in sequence to the amino acid sequence of SEQ ID NO: 51 or 98;
h) has a VH that is at least 90% (e.g, at least 92%, at least 95%, at least 98%, or at least 99%) identical in sequence to the amino acid sequence of SEQ ID NO: 52 or 99, and a VL that is at least 90% (e.g, at least 92%, at least 95%, at least 98%, or at least 99%) identical in sequence to the amino acid sequence of SEQ ID NO: 53 or 100;
i) has a VH that is at least 90% (e.g, at least 92%, at least 95%, at least 98%, or at least 99%) identical in sequence to the amino acid sequence of SEQ ID NO: 54 or 101, and a VL that is at least 90% (e.g, at least 92%, at least 95%, at least 98%, or at least 99%) identical in sequence to the amino acid sequence of SEQ ID NO: 55 or 102; or
j) has a VH that is at least 90% (e.g, at least 92%, at least 95%, at least 98%, or at least 99%) identical in sequence to the amino acid sequence of SEQ ID NO: 56, 103 or 104, and a VL that is at least 90% (e.g, at least 92%, at least 95%, at least 98%, or at least 99%) identical in sequence to the amino acid sequence of SEQ ID NO: 57 or 105.

In certain embodiments, the anti-Gal3 antibody or antigen-binding fragment thereof of the invention:
a) has a VH that comprises or consists of the amino acid sequence of SEQ ID NO: 38, 81 or 82, and a VL that comprises or consists of the amino acid sequence of SEQ ID NO: 39 or 83;
b) has a VH that comprises or consists of the amino acid sequence of SEQ ID NO: 40, 84 or 85, and a VL that comprises or consists of the amino acid sequence of SEQ ID NO: 41 or 86;
c) has a VH that comprises or consists of the amino acid sequence of SEQ ID NO: 42, 87 or 88, and a VL that comprises or consists of the amino acid sequence of SEQ ID NO: 43 or 89;
d) has a VH that comprises or consists of the amino acid sequence of SEQ ID NO: 44 or 90, and a VL that comprises or consists of the amino acid sequence of SEQ ID NO: 45 or 91;
e) has a VH that comprises or consists of the amino acid sequence of SEQ ID NO: 46 or 92, and a VL that comprises or consists of the amino acid sequence of SEQ ID NO: 47 or 93;
f) has a VH that comprises or consists of the amino acid sequence of SEQ ID NO: 48, 94 or 95, and a VL that comprises or consists of the amino acid sequence of SEQ ID NO: 49 or 96;
g) has a VH that comprises or consists of the amino acid sequence of SEQ ID NO: 50 or 97, and a VL that comprises or consists of the amino acid sequence of SEQ ID NO: 51 or 98;
h) has a VH that comprises or consists of the amino acid sequence of SEQ ID NO: 52 or 99, and a VL that comprises or consists of the amino acid sequence of SEQ ID NO: 53 or 100;
j) has a VH that comprises or consists of the amino acid sequence of SEQ ID NO: 54 or 101, and a VL that comprises or consists of the amino acid sequence of SEQ ID NO: 55 or 102; or
i) has a VH that comprises or consists of the amino acid sequence of SEQ ID NO: 56, 103 or 104, and a VL that comprises or consists of the amino acid sequence of SEQ ID NO: 57 or 105.

In certain embodiments, the anti-Gal3 antibody:
a) has an LC that comprises or consists of the amino acid sequences of SEQ ID NOs: 39 and 60; and an HC that comprises or consists of the amino acid sequences of SEQ ID NOs: 38 and 58;
b) has an LC that comprises or consists of the amino acid sequences of SEQ ID NOs: 41 and 60; and an HC that comprises or consists of the amino acid sequences of SEQ ID NOs: 40 and 58;
c) has an LC that comprises or consists of the amino acid sequences of SEQ ID NOs: 43 and 60; and an HC that comprises or consists of the amino acid sequences of SEQ ID NOs: 42 and 58;
d) has an LC that comprises or consists of the amino acid sequences of SEQ ID NOs: 45 and 60; and an HC that comprises or consists of the amino acid sequences of SEQ ID NOs: 44 and 58;
e) has an LC that comprises or consists of the amino acid sequences of SEQ ID NOs: 47 and 60; and an HC that comprises or consists of the amino acid sequences of SEQ ID NOs: 46 and 58;
f) has an LC that comprises or consists of the amino acid sequences of SEQ ID NOs: 49 and 60; and an HC that comprises or consists of the amino acid sequences of SEQ ID NOs: 48 and 58;
g) has an LC that comprises or consists of the amino acid sequences of SEQ ID NOs: 51 and 60; and an HC that comprises or consists of the amino acid sequences of SEQ ID NOs: 50 and 58;
h) has an LC that comprises or consists of the amino acid sequences of SEQ ID NOs: 53 and 60; and an HC that comprises or consists of the amino acid sequences of SEQ ID NOs: 52 and 58;
i) has an LC that comprises or consists of the amino acid sequences of SEQ ID NOs: 55 and 60; and an HC that comprises or consists of the amino acid sequences of SEQ ID NOs: 54 and 58;
j) has an LC that comprises or consists of the amino acid sequences of SEQ ID NOs: 57 and 60; and an HC that comprises or consists of the amino acid sequences of SEQ ID NOs: 56 and 58.

In certain embodiments, the anti-Gal3 antibody:
a) has an LC that comprises or consists of the amino acid sequences of SEQ ID NOs: 39 and 60; and an HC that comprises or consists of the amino acid sequences of SEQ ID NOs: 38 and 59;
b) has an LC that comprises or consists of the amino acid sequences of SEQ ID NOs: 41 and 60; and an HC that comprises or consists of the amino acid sequences of SEQ ID NOs: 40 and 59;
c) has an LC that comprises or consists of the amino acid sequences of SEQ ID NOs: 43 and 60; and an HC that comprises or consists of the amino acid sequences of SEQ ID NOs: 42 and 59;
d) has an LC that comprises or consists of the amino acid sequences of SEQ ID NOs: 45 and 60; and an HC that comprises or consists of the amino acid sequences of SEQ ID NOs: 44 and 59;
e) has an LC that comprises or consists of the amino acid sequences of SEQ ID NOs: 47 and 60; and an HC that comprises or consists of the amino acid sequences of SEQ ID NOs: 46 and 59;
f) has an LC that comprises or consists of the amino acid sequences of SEQ ID NOs: 49 and 60; and an HC that comprises or consists of the amino acid sequences of SEQ ID NOs: 48 and 59;
g) has an LC that comprises or consists of the amino acid sequences of SEQ ID NOs: 51 and 60; and an HC that comprises or consists of the amino acid sequences of SEQ ID NOs: 50 and 59;
h) has an LC that comprises or consists of the amino acid sequences of SEQ ID NOs: 53 and 60; and an HC that comprises or consists of the amino acid sequences of SEQ ID NOs: 52 and 59;
i) has an LC that comprises or consists of the amino acid sequences of SEQ ID NOs: 55 and 60; and an HC that comprises or consists of the amino acid sequences of SEQ ID NOs: 54 and 59;
j) has an LC that comprises or consists of the amino acid sequences of SEQ ID NOs: 57 and 60; and an HC that comprises or consists of the amino acid sequences of SEQ ID NOs: 56 and 59.

In certain embodiments, the anti-Gal3 antibody:
a) has an LC that comprises or consists of the amino acid sequences of SEQ ID NOs: 83 and 60; and an HC that comprises or consists of the amino acid sequences of SEQ ID NOs: 81 or 82 and of SEQ ID NOs: 58 or 59;
b) has an LC that comprises or consists of the amino acid sequences of SEQ ID NOs: 86 and 60; and an HC that comprises or consists of the amino acid sequences of SEQ ID NOs: 84 or 85 and of SEQ ID NOs: 58 or 59;
c) has an LC that comprises or consists of the amino acid sequences of SEQ ID NOs: 89 and 60; and an HC that comprises or consists of the amino acid sequences of SEQ ID NOs: 87 or 88 and of SEQ ID NOs: 58 or 59;
d) has an LC that comprises or consists of the amino acid sequences of SEQ ID NOs: 91 and 60; and an HC that comprises or consists of the amino acid sequences of SEQ ID NOs: 90 and of SEQ ID NOs: 58 or 59;
e) has an LC that comprises or consists of the amino acid sequences of SEQ ID NOs: 93 and 60; and an HC that comprises or consists of the amino acid sequences of SEQ ID NOs: 92 and of SEQ ID NOs: 58 or 59;
f) has an LC that comprises or consists of the amino acid sequences of SEQ ID NOs: 96 and 60; and an HC that comprises or consists of the amino acid sequences of SEQ ID NOs: 94 or 95 and of SEQ ID NOs: 58 or 59;
g) has an LC that comprises or consists of the amino acid sequences of SEQ ID NOs: 98 and 60; and an HC that comprises or consists of the amino acid sequences of SEQ ID NOs: 97 and of SEQ ID NOs: 58 or 59;
h) has an LC that comprises or consists of the amino acid sequences of SEQ ID NOs: 100 and 60; and an HC that comprises or consists of the amino acid sequences of SEQ ID NOs: 99 and of SEQ ID NOs: 58 or 59;
i) has an LC that comprises or consists of the amino acid sequences of SEQ ID NOs: 102 and 60; and an HC that comprises or consists of the amino acid sequences of SEQ ID NOs: 101 and of SEQ ID NOs: 58 or 59;
j) has an LC that comprises or consists of the amino acid sequences of SEQ ID NOs: 105 and 60; and an HC that comprises or consists of the amino acid sequences of SEQ ID NOs: 103 or 104 and of SEQ ID NOs: 58 or 59.

In one embodiment, the anti-Gal3 antibody competes for binding to human Gal3 with antibody D06, D11, E01, E02, E07, G03, H07, H10, B12 or E12.

In one embodiment, the anti-Gal3 antibody competes for binding to human Gal3 with antibody D06-G1, D06-G2, D11-G1, D11-G2, E01-G, E02-G, E07-G1, E07-G2, G03-G1, G03-G2, H07-G, H10-G, B12-G, E12-G1 or E12-G2.

In some embodiments, the antibody of the present invention is of isotype IgG, for example, of isotype IgG subclass IgG1 or IgG2. IgG1 constant chain sequence is provided in SEQ ID NO 58. In certain embodiments, the antibody comprises at least one mutation in the Fc region. In particular embodiments, the antibody comprises a mutation in one or more of heavy chain amino acid positions 228, 234 and 235, which are numbered according to the EU numbering or IMGT® numbering scheme. For example, one or both of the amino acid residues at positions 234 and 235 may be mutated from Leu to Ala, and/or the amino acid residue at position 228 may be mutated from Ser to Pro. Sequence of IgG1 constant chain mutated at positions 234 and 235 from Leu to Ala is provided in SEQ ID NO 59.

In another aspect, the present invention provides pharmaceutical compositions comprising at least one anti-Gal3 antibody or antigen-binding fragment thereof as described herein and a pharmaceutically acceptable excipient, optionally with an additional therapeutic agent as described herein.

The present invention further provides isolated nucleic acid molecules comprising a nucleotide sequence that encodes the heavy chain or VH of an anti-Gal3 antibody or antigen-binding fragment, comprising a nucleotide sequence that encodes the light chain or VL of an anti-Gal3 antibody or antigen-binding fragment or comprising both nucleotide sequences encoding heavy chain or VH or the light chain or VL of anti-Gal3 antibody or antigen-binding fragment thereof. The present invention further a combination of isolated nucleic acid molecules encoding an anti-Gal3 antibody or an antigen-binding fragment thereof, in particular a first isolated nucleic acid molecule comprising or consisting of a sequence encoding a heavy chain sequence or a VH sequence, and a second isolated nucleic acid molecule comprising or consisting of a sequence encoding a light chain sequence or a VL sequence.

The invention also provides vectors comprising such an isolated nucleic acid molecule or a combination of isolated nucleic acid molecules, wherein said vector may further comprise an expression control sequence.

The present invention also provides host cells comprising a nucleotide sequence that encodes the heavy chain or an antigen-binding fragment thereof, a nucleotide sequence that encodes the light chain or an antigen-binding fragment thereof, or both, of an anti-Gal3 antibody or antigen-binding fragment thereof as described herein.

The present invention also provides a method for producing an antibody or antigen-binding fragment thereof as described herein, comprising providing a host cell that comprises a nucleotide sequence that encodes the heavy chain or VH and a nucleotide sequence that encodes the light chain or VL, or that encodes both, of an anti-Gal3 antibody or antigen-binding fragment thereof as described herein, or a host cell that comprises a combination of isolated nucleic acid molecules encoding an anti-Gal3 antibody or an antigen-binding fragment thereof, in particular a first isolated nucleic acid molecule comprising or consisting of a sequence encoding a heavy chain sequence or a VH sequence, and a second isolated nucleic acid molecule comprising or consisting of a sequence encoding a light chain sequence or a VL sequence, culturing said host cell under conditions suitable for expression of the antibody or fragment thereof, and isolating the resulting recombinant antibody or fragment.

The present invention also provides a multi-specific (e.g., bi-specific) binding molecule comprising the antigen-binding fragment of an anti-Gal3 antibody described herein, and the antigen-binding fragment of another distinct antibody such as another anti-Gal3 antibody (e.g., as described herein) or an antibody that targets a different protein.

The present invention also provides a method for ameliorating the condition of a patient, preventing and/or treating a patient with a Gal3-related disorder or disease, including fibrotic diseases, inflammatory diseases, autoimmune diseases, immune-mediated disorders, neurodegenerative diseases, metabolic diseases, infectious diseases as well as several cancers, comprising administering to said patient an anti-Gal3 antibody or an antigen-binding fragment thereof, a pharmaceutical composition, or a bi-specific binding molecule as described herein. Unless otherwise indicated, a patient refers herein to a human patient.

The present invention also provides a method for inhibiting extracellular Gal3 interactions in a patient, comprising administering to said patient an anti-Gal3 antibody or an antigen-binding fragment thereof, a pharmaceutical composition, or a bi-specific binding molecule as described herein.

The present invention further provides a method for ameliorating the condition of a patient, preventing and/or treating a fibrotic disease, an inflammatory diseases, an autoimmune disease, immune-mediated disorder, a neurodegenerative disease, a metabolic disease, an infectious disease, a cancer, in a patient, comprising administering to said patient an anti-Gal3 antibody or an antigen-binding fragment thereof, a pharmaceutical composition, or a bi-specific binding molecule as described herein. In some embodiments, the fibrotic disease originates in a tissue selected from skin, lung, liver, heart, kidney and vessels.

The present invention further provides the use of an antibody composition comprising an anti-Gal3 antibody or antigen-binding fragment thereof as described herein for the manufacture of a medicament for ameliorating the condition of a patient, preventing and/or treating a patient, with a Gal3-related disease, including fibrotic diseases, such as heart failure, kidney failure, systemic sclerosis, lung fibrosis and hepatic fibrosis, and including inflammatory diseases, autoimmune diseases, immune-mediated disorders, neurodegenerative diseases, metabolic diseases, infectious diseases as well as several cancers, and/or inhibiting Gal3 interactions in a patient in need thereof.

The present invention further provides an anti-Gal3 antibody or antigen-binding fragment as described herein for ameliorating the condition of a patient, preventing and/or treating a patient with a Gal3-related disease or with a Gal3-related disorder, including fibrotic diseases, autoimmune diseases, immune-mediated disorders, neurodegenerative diseases, metabolic diseases, infectious diseases as well as several cancers, and/or inhibiting Gal3 interactions in a patient in need thereof.

The present invention further provides an article of manufacture comprising an anti-Gal3 antibody or antigen-binding fragment as described herein, wherein said article of manufacture is suitable for detecting human Gal3, or for ameliorating the condition of a patient, preventing and/or treating a patient with a Gal3-related disease, including fibrotic diseases, inflammatory diseases, autoimmune diseases, immune-mediated disorders, neurodegenerative diseases, metabolic diseases, infectious diseases as well as several cancers, and/or for inhibiting Gal3 interactions in a patient in need thereof.

### BRIEF DESCRIPTION OF THE DRAWINGS

FIG. 1 shows scFvs binding specificity by ELISA on human Gal3 full-length and CRD domain (respectively Hu-Gal3-FL, black bar, and Hu-Gal3-CRD, white bar), on human Gall (Hu-Gall, grey striped bar) and human Gal7 (Hu-Gal7, black striped bar), and on murine Gal3 (m-Gal3-FL, grey bar). Control with no antigen is represented by dotted bar (last bar on the right for each group). The first 2 bar groups are the positive and negative controls, respectively: anti-Gal3, anti-Gall and anti-Gal7 commercial antibodies to confirm the coating of the five antigens tested, and an irrelevant scFv tested on the 5 antigens.

FIG. 2 shows inhibition of Gal3-ASF complex formation by scFv expressed in the supernatant by competitive ELISA. Absorbance results were expressed in percentages where ASF with an irrelevant scFv corresponds to 100% signal (upper dashed line), and the lower dashed line corresponds to the signal with no binding, i.e. 100% inhibition (secondary antibody alone). Data are representative of three independent experiments and values are expressed in mean±SEM.

FIG. 3 shows human and mouse cross-reactivity by ELISA on Hu-Gal3-FL (1^{st} white bar), Hu-Gal3-CRD (2^{nd} grey bar), and m-Gal3-FL (3^{rd} striped bar). The first 2 groups are the controls, respectively: commercial anti-Gal3 antibody to confirm the coating of Hu-Gal3 antigen, and an irrelevant IgG tested on the 3 antigens.

FIG. 4 shows the absence of binding of anti-Gal3 antibodies to Hu-Gall (2^{nd} black bar) and Hu-Gal7 (3^{rd} striped bar) compared to the positive control to Hu-Gal3-CRD (1^{st} white bar). The first 3 bar groups are the positive and negative controls, respectively: anti-Gall and anti-Gal7 antibodies to confirm the coating of the two antigens tested and an irrelevant IgG tested on the 3 conditions.

FIG. 5 shows antibody binding to Hu-Gal3-FL and m-Gal3-FL in circle and triangle respectively. The EC50 are the best fit values (Hill equation).

FIG. 6 shows the dose-response ELISA inhibition of ASF binding to Hu-Gal3-CRD by the anti-Gal3 antibodies. Measured (triangle) and fitted (curve) OD450 nm for each anti-Gal3 antibody concentration are in black. Measured (circles) OD450 nm for the irrelevant antibody is in grey.

FIG. 7 shows the evolution of skin thickness variation during two independent studies, comparing to control group mice treated with TD139, E07 and D11 antibodies in one study (FIG. 7A) and TD139, E07 and H10 antibodies in the other study (FIG. 7B). Data are quoted as means±SEM. * p<0.05, ** p<0.01, ***p<0.001 compared to HOCI group. ###p<0.001 compared to control group. Repeated measures ANOVA with Dunnett's post hoc test per day.

FIG. 8 shows histograms depicting Image J software-assisted quantification of collagen deposition in Picrosirius red stained lung (FIG. 8A) and skin (FIG. 8B) sections. Data are expressed as means±SEM. * p<0.05, ** p<0.01, compared to HOCI group. ###p<0.001 compared to control group. Mixed 1 way-ANOVA (mice as random effect) with Dunnett's post hoc test.

FIG. 9 shows histograms depicting IL-5 and IL-6 levels in plasma at day 42 in control group, HOCI group and HOCI antibody- or TD139-treated groups. Data are expressed as means±SEM. ** p<0.01 compared to HOCI group. ##p<0.01 compared to control group. Repeated measures ANCOVA (day minus 1 measurement as covariate) on log-transformed data with Dunnett's post hoc test per day.

FIG. 10 shows histograms depicting Gal3 quantification in mice plasma before test item administration (day 0) and at day 42, in control group, HOCI group and HOCI antibody- or TD139-treated groups. Data are expressed as means±SEM. ***p<0.001 compared to HOCL group. ##p<0.01 compared to control group. Mixed ANCOVA (day minus 1 measurement as covariate and mice as random effect) with Dunnett's post hoc test.

FIG. 11 shows a heatmap representation of the genes with differential expression at day 42 in whole blood cells, using RNA sequencing methodology in control group, HOCI group, HOCI E07 antibody-treated group and TD139-treated group.

FIG. 12 shows the change in body weight in bleomycin treated mice with or without treatment with D06, D11, E07 or TD139 from day minus 1 to day 21.

FIG. 13 shows the time to weight growth (TWG) and the time to baseline (TTB) in bleomycin-treated mice with or without treatment with D06, D11, E07 or TD139. Data are expressed as individual values predicted by a non linear model and median with interquartiles. * p<0.05, ** p<0.01 compared to bleo group. Logrank test followed by Bonferroni-Holm adjustment for multiplicity.

FIG. 14 shows a picture representing the sections made on lungs before staining with picrosirius red and scoring with the use of the Modified Ashcroft fibrosis score.

FIG. 15 shows the frequency of median lung fibrosis Ashcroft scores in bleomycin-treated mice without any treatment or with a treatment with D06 antibody, D11 antibody, E07 antibody or TD139.

FIG. 16 shows scatter dot plot of neutrophils counts in Bronchoalveolar Lavage fluid of rats from Acute Respiratory Distress Syndrome (ARDS) model. Control with no LPS challenge and no treatment is represented by black circle, positive control with LPS challenge but no treatment is represented by black square and group with LPS Challenge and dexamethasone (Dexa) as reference treatment is depicted as white square. Groups treated by anti-Gal3 antibodies prior to LPS challenge are depicted by black triangle for D11 antibody, inversed black triangle for D06 antibody, black diamond for E07 antibody and white circle for H10 antibody. Data are expressed as means+/−SEM. ####p<0.0001 between LPS and CTRL group; ** p<0.01; **** p<0.0001 compared to positive control LPS group. One-way ANOVA on log-transformed data using Dunnett adjustment method.

FIG. 17 shows scatter dot plot of protein quantification in Bronchoalveolar Lavage fluid of rats from the Acute Respiratory Distress Syndrome (ARDS) model. Control with no LPS challenge and no treatment is represented by black circle, positive control with LPS challenge but no treatment is represented by black square and group with LPS Challenge and dexamethasone (Dexa) as reference treatment is depicted as white square. Groups treated by anti-Gal3 antibodies prior to LPS challenge are depicted by black triangle for D11 antibody, inversed black triangle for D06 antibody, black diamond for E07 antibody and white circle for H10 antibody. Data are expressed as means+/−SEM. #p<0.05 between LPS and CTRL group; ** p<0.051 compared to positive control LPS group. One-way ANOVA on log-transformed data using Dunnett adjustment method.

FIG. 18 shows scatter dot plot of TNF alpha quantification by MSD in the plasma of rats from the Acute Respiratory Distress Syndrome (ARDS) model. Control with no LPS challenge and no treatment is represented by black circle, positive control with LPS challenge but no treatment is represented by black square and group with LPS Challenge and dexamethasone (Dexa) as reference treatment is depicted as white square. Groups treated by anti-Gal3 antibodies prior to LPS challenge are depicted by black triangle for D11 antibody, inversed black triangle for D06 antibody, black diamond for E07 antibody and white circle for H10 antibody. Data are expressed as means+/−SEM. ####p<0.0001 between LPS and CTRL group; ** p<0.01; **** p<0.0001 compared to positive control LPS group. One-way ANOVA on log-transformed data using Dunnett adjustment method.

FIG. 19 shows scatter dot plot of KC-GRO quantification by MSD in the plasma of rats from the Acute Respiratory Distress Syndrome (ARDS) model. Control with no LPS challenge and no treatment is represented by black circle, positive control with LPS challenge but no treatment is represented by black square and group with LPS Challenge and dexamethasone (Dexa) as reference treatment is depicted as white square. Groups treated by anti-Gal3 antibodies prior to LPS challenge are depicted by black triangle for D11 antibody, inversed black triangle for D06 antibody, black diamond E07 antibody and white circle for H10 antibody. Data are expressed as means+/−SEM. ####p<0.0001 between LPS and CTRL group; **** other p<0.0001 values as indicated compared to positive control LPS group. One-way ANOVA on log-transformed data using Dunnett adjustment method.

### DETAILED DESCRIPTION OF THE INVENTION

The present invention provides new anti-human Galectin-3 (Gal3) antibodies. A human Gal3 polypeptide sequence is available under UniProt Accession No. P17931 (LEG3_HUMAN).

The present invention is directed to novel monoclonal antibodies targeting extracellular Galectin-3 (Gal3), and binding neither to Galectin-1 (Gall) nor Galectin-7 (Gal7), and to pharmaceutical compositions comprising one or more of these antibodies.

The present invention is also directed to the use of said anti-Gal3 antibodies and pharmaceutical compositions for inhibiting Gal3 interactions in a patient in need thereof or for ameliorating, preventing and/or treating a patient with a Gal3-related disease or a Gal3-related disorder, including fibrotic diseases, inflammatory diseases, autoimmune diseases, immune-mediated disorders, neurodegenerative diseases, metabolic diseases, infectious diseases as well as several cancers.

Tissue fibrosis is a progressive debilitating disease characterized by an abundant accumulation of extracellular matrix proteins such as collagens and fibronectin, leading to tissue scarring, organ injury, organ function decline, and subsequent organ failure. Tissue fibrosis can be located in the kidney, liver, lung, heart, skin, pancreas, intestine, eye, nervous system, joint, tendon, mediastinum, or retroperitoneum. Features of tissue fibrosis comprise epithelial and endothelial injury and dysfunction, abnormal proliferation of myofibroblasts, smooth muscle cells and stellate cells, and extracellular matrix proteins deposition. The presence of cytokines, chemokines, growth factors, and angiogenic factors further regulate the activation of the extracellular matrix proteins producing cells during profibrotic process.

The term “antibody” (Ab) or “immunoglobulin” (Ig), as used herein, refers to a tetramer comprising two heavy (H) chains (about 50-70 kDa) and two light (L) chains (about 25 kDa) inter-connected by disulfide bonds. Each heavy chain is comprised of a heavy chain variable domain (VH) and a heavy chain constant region (CH). Each light chain is composed of a light chain variable domain (VL) and a light chain constant region (CL). The VH and VL domains can be subdivided further into regions of hypervariability, termed “complementarity determining regions” (CDRs), interspersed with regions that are more conserved, termed “framework regions” (FRs). Each VH and VL is composed of three CDRs (H-CDR herein designates a CDR from the heavy chain; and L-CDR herein designates a CDR from the light chain) and four FRs, arranged from amino-terminus to carboxyl-terminus in the following order: FR1, CDR1, FR2, CDR2, FR3, CDR3, FR4. The assignment of amino acid numbers in the heavy or light chain may be in accordance with EU numbering or IMGT® definitions (Lefranc et al., Dev Comp Immunol 27(1):55-77 (2003)); or the definitions of Kabat, Sequences of Proteins of Immunological Interest (National Institutes of Health, Bethesda, MD (1987 and 1991)); Chothia & Lesk, J. Mol. Biol. 196:901-917 (1987); or Chothia et al., Nature 342:878-883 (1989). The term “recombinant antibody” refers to an antibody that is expressed from a cell or cell line comprising the nucleotide sequence(s) that encode the antibody, wherein said nucleotide sequence(s) are not naturally associated with the cell.

The term “isolated protein”, “isolated polypeptide” or “isolated antibody” refers to a protein, polypeptide or antibody that by virtue of its origin or source of derivation (1) is not associated with naturally associated components that accompany it in its native state, (2) is free of other proteins from the same species, (3) is expressed by a cell from a different species, and/or (4) does not occur in nature. Thus, a polypeptide that is chemically synthesized or synthesized in a cellular system different from the cell from which it naturally originates will be “isolated” from its naturally associated components. A protein may also be rendered substantially free of naturally associated components by isolation, using protein purification techniques well known in the art.

As used herein, the term “germline” refers to the nucleotide and amino acid sequences of antibody genes and gene segments as they are passed from parents to offspring via germ cells. Germline sequences are distinguished from the nucleotide sequences encoding antibodies in mature B cells, which have been altered by recombination and hypermutation events during the course of B cell maturation. An antibody that “utilizes” a particular germline sequence has a nucleotide or amino acid sequence that aligns with that germline nucleotide sequence or with the amino acid sequence that it specifies more closely than with any other germline nucleotide or amino acid sequence.

The term “affinity” refers to a measure of the attraction between an antigen and an antibody. The intrinsic attractiveness of the antibody for the antigen is typically expressed as the binding affinity equilibrium constant (KD) of a particular antibody-antigen interaction. An antibody is said to specifically bind to an antigen when the KD is ≤1 mM, preferably ≤100 nM. A KD binding affinity constant can be measured, e.g., by surface plasmon resonance (BIAcore™) or Bio-Layer Interferometry, for example using the IBIS MX96 SPR system from IBIS Technologies or the Octet™ system from ForteBio.

The term “humanize” refers to the fact that where an antibody is wholly or partially of non-human origin (for example, a murine or chicken antibody obtained from immunization of mice or chickens, respectively, with an antigen of interest, or a chimeric antibody based on such a murine or chicken antibody), it is possible to replace certain amino acids, in particular in the framework regions and constant regions of the heavy and light chains, in order to avoid or minimize an immune response in humans. Although it is not possible to precisely predict the immunogenicity, and thereby the human anti-antibody response, of a particular antibody, non-human antibodies tend to be more immunogenic in humans than human antibodies. Chimeric antibodies, where the foreign (e.g. rodent or avian) constant regions have been replaced with sequences of human origin, have been shown to be generally less immunogenic than antibodies of fully foreign origin, and the trend in therapeutic antibodies is towards humanized or fully human antibodies. Chimeric antibodies or other antibodies of non-human origin thus can be humanized to reduce the risk of a human anti-antibody response.

Numerous methods for humanization of an antibody sequence are known in the art; see, e.g., the review by Almagro & Fransson, Front Biosci. 13:1619-1633 (2008). One commonly used method is CDR grafting, which for, e.g., a murine-derived chimeric antibody involves identification of human germline gene counterparts to the murine variable domain genes and grafting of the murine CDR sequences into this framework. The specificity of an antibody's interaction with a target antigen resides primarily in the amino acid residues located in the six CDRs of the heavy and light chain.

The amino acid sequences within CDRs are therefore much more variable between individual antibodies than sequences outside of CDRs. Because CDR sequences are responsible for most antibody-antigen interactions, it is possible to express recombinant antibodies that mimic the properties of a specific naturally occurring antibody, or more generally any specific antibody with a given amino acid sequence, e.g., by constructing expression vectors that express CDR sequences from the specific antibody grafted into framework sequences from a different antibody. As a result, it is possible to “humanize” a non-human antibody and still substantially maintain the binding specificity and affinity of the original antibody. CDR grafting may be based on the Kabat CDR definitions, although a more recent publication (Magdelaine-Beuzelin et al., Crit Rev. Oncol Hematol. 64:210-225 (2007)) has suggested that the IMGT® definition (the international ImMunoGeneTics information System®, www.imgt.org) (or EU numbering) may improve the result of the humanization (see Lefranc et al., Dev. Comp Immunol. 27:55-77 (2003)).

In some cases, CDR grafting may reduce the binding specificity and affinity, and thus the biological activity, of a CDR-grafted antibody as compared to the parent antibody from which the CDRs are obtained. Back mutations (sometimes referred to as “framework repair”) may be introduced at selected positions of the CDR-grafted antibody, typically in the framework regions, in order to reestablish the binding specificity and affinity of the parent antibody. Positions for possible back mutations can be identified using information available in the literature and in antibody databases. Amino acid residues that are candidates for back mutations are typically those that are located at the surface of an antibody molecule, while residues that are buried or that have a low degree of surface exposure will not normally be altered. An alternative humanization technique to CDR grafting and back mutation is resurfacing, in which non-surface exposed residues of non-human origin are retained, while surface residues are altered to human residues.

In certain cases, it also may be desirable to alter one or more CDR amino acid residues in order to improve binding affinity to the target epitope. This is known as “affinity maturation” and may optionally be performed in connection with humanization, for example in situations where humanization of an antibody leads to reduced binding specificity or affinity and it is not possible to sufficiently improve the binding specificity or affinity by back mutations alone. Various affinity maturation methods are known in the art, for example the in vitro scanning saturation mutagenesis method described by Burks et al., Proc Natl Acad Sci USA, 94:412-417 (1997), and the stepwise in vitro affinity maturation method of Wu et al., Proc Natl Acad Sci USA 95:6037-6042 (1998).

The term “antigen-binding fragment” of an antibody (or simply “antibody fragment”), as used herein, refers to one or more fragments or fragments of an antibody that retain the ability to specifically bind to an antigen (e.g., human Gal3, or a fragment thereof). It has been shown that certain fragments of a full-length antibody can perform the antigen-binding function of the antibody. Examples of binding fragments encompassed within the term “antigen-binding fragment” include (i) a Fab fragment: a monovalent fragment consisting of the VL, VH, CL and CH1 domains; (ii) a F(ab′)2 fragment: a bivalent fragment comprising two Fab fragments linked by a disulfide bridge at the hinge region; (iii) an Fd fragment consisting of the VH and CH1 domains; (iv) a Fv fragment consisting of the VL and VH domains of a single arm of an antibody, (v) a dAb fragment, which consists of a VH domain; and (vi) an isolated complementarity determining region (CDR) capable of specifically binding to an antigen. Furthermore, although the two domains of the Fv fragment, VL and VH, are encoded by separate genes, they can be joined, using recombinant methods, by a synthetic linker that enables them to be made as a single protein chain in which the VL and VH domains pair to form monovalent molecules (known as single chain Fv (scFv or scFvs)). Also within the invention are antigen-binding molecules comprising a VH and/or a VL. In the case of a VH, the molecule may also comprise one or more of a CH1, hinge, CH2, or CH3 region. Such single chain antibodies are also intended to be encompassed within the term “antigen-binding fragment” of an antibody. Other forms of single chain antibodies, such as diabodies, are also encompassed. Diabodies are bivalent, bi-specific antibodies in which VH and VL domains are expressed on a single polypeptide chain, but using a linker that is too short to allow for pairing between the two domains on the same chain, thereby forcing the domains to pair with complementary domains of another chain and creating two antigen-binding sites.

Antibody fragments, such as Fab and F(ab′)2 fragments, can be prepared from whole antibodies using conventional techniques, such as papain or pepsin digestion of whole antibodies. Moreover, antibodies, antibody fragments and immunoadhesion molecules can be obtained using standard recombinant DNA techniques, e.g., as described herein.

The class (isotype) and subclass of anti-Gal3 antibodies may be determined by any method known in the art. In general, the class and subclass of an antibody may be determined using antibodies that are specific for a particular class and subclass of antibody. Such antibodies are available commercially. The class and subclass can be determined by ELISA, Western Blot as well as other techniques. Alternatively, the class and subclass may be determined by sequencing all or a fragment of the constant regions of the heavy and/or light chains of the antibodies, comparing their amino acid sequences to the known amino acid sequences of various classes and subclasses of immunoglobulins, and determining the class and subclass of the antibodies.

Unless otherwise indicated, all antibody amino acid residue numbers referred to in this disclosure are those under the EU numbering or IMGT® numbering scheme.

### Anti-Gal3 Antibodies

The present invention provides antibodies directed against Gal3, and antigen-binding fragments thereof. One advantage of the novel anti-Gal3 antibodies of the invention is that they are able to bind selectively to Gal3, particularly to Gal3 CRD, but neither to Gall nor to Gal7 (see Example 4). They are able to inhibit Gal3 binding to its ligands (see Example 6). Another potential advantage of the anti-Gal3 antibodies of the invention is a low level of secondary effector functions in antibodies having the “LALA” mutations (L234A/L235A), which hinder significant antibody binding to human FcgR (Fc gamma receptors) and hence depletion of effector T-cells. In another embodiment, the present invention provides antibodies with framework mutations to germline in the variable domains of the antibodies outside the CDR regions, allowing potentially reduced immunogenicity when administered to humans, while substantially retaining the specificity and affinity of the parental antibodies.

In one embodiment, the anti-Gal3 antibody or antigen-binding fragment thereof has a heavy chain variable domain (VH) that is at least 90% identical in sequence to any one of SEQ ID NO: 38, 40, 42, 44, 46, 48, 50, 52, 54, 56, 81, 82, 84, 85, 87, 88, 90, 92, 94, 95, 97, 99, 101, 103 or 104, e.g. at least 92% identical, such as at least 95%, at least 96%, at least 97%, at least 98% or at least 99% identical in sequence to the VH amino acid sequence of SEQ ID NO: 38, 40, 42, 44, 46, 48, 50, 52, 54, 56, 81, 82, 84, 85, 87, 88, 90, 92, 94, 95, 97, 99, 101, 103 or 104.

In one embodiment, the anti-Gal3 antibody has a heavy chain variable domain (VH) that is at least 90% identical in sequence to any one of SEQ ID NO: 38, 40, 42, 44, 46, 48, 50, 52, 54, 56, 81, 82, 84, 85, 87, 88, 90, 92, 94, 95, 97, 99, 101, 103 or 104, e.g. at least 92% identical, such as at least 95%, at least 96%, at least 97%, at least 98% or at least 99% identical in sequence to the VH amino acid sequence of SEQ ID NO: 38, 40, 42, 44, 46, 48, 50, 52, 54, 56, 81, 82, 84, 85, 87, 88, 90, 92, 94, 95, 97, 99, 101, 103 or 104; and a heavy chain constant region that is at least 90% identical in sequence to SEQ ID NO: 58 or 59, e.g. at least 92% identical, such as at least 95%, at least 96%, at least 97%, at least 98% or at least 99% identical to SEQ ID NO: 58 or 59.

In one embodiment, the anti-Gal3 antibody has a heavy chain (HC) that comprises the VH amino acid sequence of any one of SEQ ID NO: 38, 40, 42, 44, 46, 48, 50, 52, 54, 56, 81, 82, 84, 85, 87, 88, 90, 92, 94, 95, 97, 99, 101, 103 or 104 and the heavy chain constant region amino acid sequence of SEQ ID NO: 58 or 59.

In one embodiment, the anti-Gal3 antibody or antigen-binding fragment thereof has a light chain variable domain (VL) that is at least 90% identical in sequence to the VL amino acid sequence of any one of SEQ ID NO: 39, 41, 43, 45, 47, 49, 51, 53, 55, 57, 83, 86, 89, 91, 93, 96, 98, 100, 102 or 105, e.g. at least 92% identical, such as at least 95%, at least 96%, at least 97%, at least 98% or at least 99% identical to any one of SEQ ID NO: 39, 41, 43, 45, 47, 49, 51, 53, 55, 57, 83, 86, 89, 91, 93, 96, 98, 100, 102 or 105.

In one embodiment, the anti-Gal3 antibody has a light chain variable domain (VL) that is at least 90% identical in sequence to the VL amino acid sequence of any one of SEQ ID NO: 39, 41, 43, 45, 47, 49, 51, 53, 55, 57, 83, 86, 89, 91, 93, 96, 98, 100, 102 or 105, e.g. at least 92% identical, such as at least 95%, at least 96%, at least 97%, at least 98% or at least 99% identical to any one of SEQ ID NO: 39, 41, 43, 45, 47, 49, 51, 53, 55, 57, 83, 86, 89, 91, 93, 96, 98, 100, 102 or 105; and a light chain constant region amino acid sequence that is at least 90% identical in sequence to SEQ ID NO: 60, e.g. at least 92% identical, such as at least 95%, at least 96%, at least 97%, at least 98% or at least 99% identical to SEQ ID NO: 60.

In another embodiment, the anti-Gal3 antibody has a light chain that comprises any one of SEQ ID NO: 39, 41, 43, 45, 47, 49, 51, 53, 55, 57, 83, 86, 89, 91, 93, 96, 98, 100, 102 or 105, and SEQ ID NO: 60.

In certain embodiments, the anti-Gal3 antibody or antigen-binding fragment of the invention comprises the H-CDR 1-3 and L-CDR 1-3 amino acid sequences of:
a) SEQ ID NOs: 1, 6, 12, 20, 27 and 30, respectively;
b) SEQ ID NOs: 1, 6, 13, 21, 27 and 31, respectively;
c) SEQ ID NOs: 1, 6, 14, 22, 27 and 31, respectively;
d) SEQ ID NOs: 5, 7, 3, 21, 28 and 32, respectively;
e) SEQ ID NOs: 3, 8, 15, 23, 27 and 33, respectively;
f) SEQ ID NOs: 2, 6, 16, 24, 27 and 34, respectively;
g) SEQ ID NOs: 1, 9, 14, 24, 27 and 34, respectively;
h) SEQ ID NOs: 2, 10, 17, 25, 27 and 35, respectively;
i) SEQ ID NOs: 4, 11, 18, 21, 29 and 36, respectively; or
j) SEQ ID NOs: 2, 10, 19, 26, 27 and 37, respectively.

In some embodiments, the anti-Gal3 antibody or antigen-binding fragment of the invention comprises a VH and a VL that are 80%, 85%, 90%, 91%, 92%, 93%, 94%, 95%, 96%, 97%, 98%, or 99% identical to the amino acid sequences of:
a) SEQ ID NOs: 38 and 39, respectively;
b) SEQ ID NOs: 40 and 41, respectively;
c) SEQ ID NOs: 42 and 43, respectively;
d) SEQ ID NOs: 44 and 45, respectively;
e) SEQ ID NOs: 46 and 47, respectively;
f) SEQ ID NOs: 48 and 49, respectively;
g) SEQ ID NOs: 50 and 51, respectively;
h) SEQ ID NOs: 52 and 53, respectively;
i) SEQ ID NOs: 54 and 55, respectively;
j) SEQ ID NOs: 56 and 57, respectively;
k) SEQ ID NOs: 81 and 83, respectively;
l) SEQ ID NOs: 82 and 83, respectively;
m) SEQ ID NOs: 84 and 86, respectively;
n) SEQ ID NOs: 85 and 86, respectively;
o) SEQ ID NOs: 87 and 89, respectively;
p) SEQ ID NOs: 88 and 89, respectively;
q) SEQ ID NOs: 90 and 91, respectively;
r) SEQ ID NOs: 92 and 93, respectively;
s) SEQ ID NOs: 94 and 96, respectively;
t) SEQ ID NOs: 95 and 96, respectively;
u) SEQ ID NOs: 97 and 98, respectively;
v) SEQ ID NOs: 99 and 100, respectively;
w) SEQ ID NOs: 101 and 102, respectively;
x) SEQ ID NOs: 103 and 105, respectively; or
y) SEQ ID NOs: 104 and 105, respectively.

In some embodiments, the anti-Gal3 antibody or antigen-binding fragment of the invention comprises a VH and a VL that have the amino acid sequences of:
a) SEQ ID NOs: 38 and 39, respectively;
b) SEQ ID NOs: 40 and 41, respectively;
c) SEQ ID NOs: 42 and 43, respectively;
d) SEQ ID NOs: 44 and 45, respectively;
e) SEQ ID NOs: 46 and 47, respectively;
f) SEQ ID NOs: 48 and 49, respectively;
g) SEQ ID NOs: 50 and 51, respectively;
h) SEQ ID NOs: 52 and 53, respectively;
i) SEQ ID NOs: 54 and 55, respectively;
j) SEQ ID NOs: 56 and 57, respectively;
k) SEQ ID NOs: 81 and 83, respectively;
l) SEQ ID NOs: 82 and 83, respectively;
m) SEQ ID NOs: 84 and 86, respectively;
n) SEQ ID NOs: 85 and 86, respectively;
o) SEQ ID NOs: 87 and 89, respectively;
p) SEQ ID NOs: 88 and 89, respectively;
q) SEQ ID NOs: 90 and 91, respectively;
r) SEQ ID NOs: 92 and 93, respectively;
s) SEQ ID NOs: 94 and 96, respectively;
t) SEQ ID NOs: 95 and 96, respectively;
u) SEQ ID NOs: 97 and 98, respectively;
v) SEQ ID NOs: 99 and 100, respectively;
w) SEQ ID NOs: 101 and 102, respectively;
x) SEQ ID NOs: 103 and 105, respectively; or
y) SEQ ID NOs: 104 and 105, respectively.

In certain embodiments, the anti-Gal3 antibody:
a) has an LC that comprises or consists of the amino acid sequences of SEQ ID NOs: 39 and 60; and an HC that comprises or consists of the amino acid sequences of SEQ ID NO: 38 and of SEQ ID NOs: 58 or 59;
b) has an LC that comprises or consists of the amino acid sequences of SEQ ID NOs: 41 and 60; and an HC that comprises or consists of the amino acid sequences of SEQ ID NO: 40 and of SEQ ID NOs: 58 or 59;
c) has an LC that comprises or consists of the amino acid sequences of SEQ ID NOs: 43 and 60; and an HC that comprises or consists of the amino acid sequences of SEQ ID NO: 42 and of SEQ ID NOs: 58 or 59;
d) has an LC that comprises or consists of the amino acid sequences of SEQ ID NOs: 45 and 60; and an HC that comprises or consists of the amino acid sequences of SEQ ID NO: 44 and of SEQ ID NOs: 58 or 59;
e) has an LC that comprises or consists of the amino acid sequences of SEQ ID NOs: 47 and 60; and an HC that comprises or consists of the amino acid sequences of SEQ ID NO: 46 and of SEQ ID NOs: 58 or 59;
f) has an LC that comprises or consists of the amino acid sequences of SEQ ID NOs: 49 and 60; and an HC that comprises or consists of the amino acid sequences of SEQ ID NO: 48 and of SEQ ID NOs: 58 or 59;
g) has an LC that comprises or consists of the amino acid sequences of SEQ ID NOs: 51 and 60; and an HC that comprises or consists of the amino acid sequences of SEQ ID NO: 50 and of SEQ ID NOs: 58 or 59;
h) has an LC that comprises or consists of the amino acid sequences of SEQ ID NOs: 53 and 60; and an HC that comprises or consists of the amino acid sequences of SEQ ID NO: 52 and of SEQ ID NOs: 58 or 59;
i) has an LC that comprises or consists of the amino acid sequences of SEQ ID NOs: 55 and 60; and an HC that comprises or consists of the amino acid sequences of SEQ ID NO: 54 and of SEQ ID NOs: 58 or 59;
j) has an LC that comprises or consists of the amino acid sequences of SEQ ID NOs: 57 and 60; and an HC that comprises or consists of the amino acid sequences of SEQ ID NO: 56 and of SEQ ID NOs: 58 or 59.

In certain embodiments, the anti-Gal3 antibody:
a) has an LC that comprises or consists of the amino acid sequences of SEQ ID NOs: 83 and 60; and an HC that comprises or consists of the amino acid sequences of SEQ ID NOs: 81 or 82 and of SEQ ID NOs: 58 or 59;
b) has an LC that comprises or consists of the amino acid sequences of SEQ ID NOs: 86 and 60; and an HC that comprises or consists of the amino acid sequences of SEQ ID NOs: 84 or 85 and of SEQ ID NOs: 58 or 59;
c) has an LC that comprises or consists of the amino acid sequences of SEQ ID NOs: 89 and 60; and an HC that comprises or consists of the amino acid sequences of SEQ ID NOs: 87 or 88 and of SEQ ID NOs: 58 or 59;
d) has an LC that comprises or consists of the amino acid sequences of SEQ ID NOs: 91 and 60; and an HC that comprises or consists of the amino acid sequences of SEQ ID NOs: 90 and of SEQ ID NOs: 58 or 59;
e) has an LC that comprises or consists of the amino acid sequences of SEQ ID NOs: 93 and 60; and an HC that comprises or consists of the amino acid sequences of SEQ ID NOs: 92 and of SEQ ID NOs: 58 or 59;
f) has an LC that comprises or consists of the amino acid sequences of SEQ ID NOs: 96 and 60; and an HC that comprises or consists of the amino acid sequences of SEQ ID NOs: 94 or 95 and of SEQ ID NOs: 58 or 59;
g) has an LC that comprises or consists of the amino acid sequences of SEQ ID NOs: 98 and 60; and an HC that comprises or consists of the amino acid sequences of SEQ ID NOs: 97 and of SEQ ID NOs: 58 or 59;
h) has an LC that comprises or consists of the amino acid sequences of SEQ ID NOs: 100 and 60; and an HC that comprises or consists of the amino acid sequences of SEQ ID NOs: 99 and of SEQ ID NOs: 58 or 59;
i) has an LC that comprises or consists of the amino acid sequences of SEQ ID NOs: 102 and 60; and an HC that comprises or consists of the amino acid sequences of SEQ ID NOs: 101 and of SEQ ID NOs: 58 or 59;
j) has an LC that comprises or consists of the amino acid sequences of SEQ ID NOs: 105 and 60; and an HC that comprises or consists of the amino acid sequences of SEQ ID NOs: 103 or 104 and of SEQ ID NOs: 58 or 59.

In some embodiments, the anti-Gal3 antibody or antigen-binding fragment of the invention comprises the H-CDR 1-3 and L-CDR 1-3 amino acid sequences of antibody D06, D11, E01, E02, E07, G03, H07, H10, B12 or E12.

In some embodiments, the anti-Gal3 antibody or antigen-binding fragment of the invention comprises a VH and VL that are at least 90% identical in amino acid sequence to the VH and VL respectively of antibody D06, D11, E01, E02, E07, G03, H07, H10, B12 or E12.

In some embodiments, the anti-Gal3 antibody or antigen-binding fragment of the invention comprises a VH and VL that are the VH and VL respectively of antibody D06, D11, E01, E02, E07, G03, H07, H10, B12 or E12.

In some embodiments, the anti-Gal3 antibody or antigen-binding fragment of the invention comprises a VH and VL that are at least 90% identical in amino acid sequence to the VH and VL respectively of antibody D06-G1, D06-G2, D11-G1, D11-G2, E01-G, E02-G, E07-G1, E07-G2, G03-G1, G03-G2, H07-G, H10-G, B12-G, E12-G1 or E12-G2.

In some embodiments, the anti-Gal3 antibody or antigen-binding fragment of the invention comprises a VH and VL that are the VH and VL respectively of antibody D06, D11, E01, E02, E07, G03, H07, H10, B12, E12, D06-G1, D06-G2, D11-G1, D11-G2, E01-G, E02-G, E07-G1, E07-G2, G03-G1, G03-G2, H07-G, H10-G, B12-G, E12-G1 or E12-G2.

In some embodiments, the anti-Gal3 antibody or antigen-binding fragment thereof of the invention is the antibody D06, D11, E01, E02, E07, G03, H07, H10, B12, E12, D06-G1, D06-G2, D11-G1, D11-G2, E01-G, E02-G, E07-G1, E07-G2, G03-G1, G03-G2, H07-G, H10-G, B12-G, E12-G1 or E12-G2, or antigen-binding fragment thereof or an antibody or antigen-binding fragment thereof with the same amino acid sequences as said antibody or antigen-binding fragment thereof.

In some embodiments, any of the anti-Gal3 antibodies or antigen-binding fragment thereof do not bind to Gall or Gal7.

In some embodiments, any of the anti-Gal3 antibodies or antigen-binding fragment thereof may inhibit and/or reduce Gal3 expression and/or Gal3 interactions with its ligands, allowing amelioration, prevention and/or treatment of a Gal3-related disease or Gal3-related disorder.

In some embodiments, any of the anti-Gal3 antibodies or antigen-binding fragment thereof may inhibit IL-6 and/or IL-5 levels in plasma.

In some embodiments, any of the anti-Gal3 antibodies or antigen-binding fragment thereof may reduce skin thickness or collagen deposit in fibrotic disease affected patient.

The class of an anti-Gal3 antibody obtained by the methods described herein may be changed or switched with another class or subclass. In one aspect of the invention, a nucleic acid molecule encoding VL or VH is isolated using methods well-known in the art such that it does not include nucleic acid sequences encoding CL or CH, respectively. The nucleic acid molecules encoding VL or VH then are operatively linked to a nucleic acid sequence encoding a CL or CH, respectively, from a different class of immunoglobulin molecule. This may be achieved using a vector or nucleic acid molecule that comprises a CL or CH chain, as described above. For example, an anti-Gal3 antibody that was originally IgM may be class switched to IgG. Further, the class switching may be used to convert one IgG subclass to another, e.g., from IgG1 to IgG2 or from IgG1 to IgG4. A κ light chain constant region can be changed, e.g., to a λ light chain constant region. A preferred method for producing an antibody of the invention with a desired Ig isotype comprises the steps of isolating a nucleic acid molecule encoding the heavy chain of an anti-Gal3 antibody and a nucleic acid molecule encoding the light chain of an anti-Gal3 antibody, obtaining the variable domain of the heavy chain, ligating the variable domain of the heavy chain with the constant region of a heavy chain of the desired isotype, expressing the light chain and the ligated heavy chain in a cell, and collecting the anti-Gal3 antibody with the desired isotype.

The anti-Gal3 antibody of the invention can be an IgG, an IgM, an IgE, an IgA, or an IgD molecule, but is typically of the IgG isotype, e.g. of IgG subclass IgG1, IgG2a or IgG2b, IgG3 or IgG4. In one embodiment, the antibody is an IgG1. In another embodiment, the antibody is an IgG2 or an IgG4.

In one embodiment, the anti-Gal3 antibody may comprise at least one mutation in the Fc region. A number of different Fc mutations are known, where these mutations provide altered effector function. For example, in many cases it will be desirable to reduce or eliminate effector function, e.g. where ligand/receptor interactions are undesired or in the case of antibody-drug conjugates.

In one embodiment, the anti-Gal3 antibody comprises at least one mutation in the Fc region that reduces effector function. Fc region amino acid positions that may be advantageous to mutate in order to reduce effector function include one or more of positions 228, 233, 234 and 235, where amino acid positions are numbered according to the EU numbering or IMGT® numbering scheme.

In one embodiment, one or both of the amino acid residues at positions 234 and 235 may be mutated, for example from Leu to Ala (L234A/L235A).

These mutations reduce effector function of the Fc region of IgG1 antibodies. The sequence of such mutated IgG1 constant chain is provided in SEQ ID NO 59. Additionally, or alternatively, the amino acid residue at position 228 may be mutated, for example to Pro. In some embodiments, the amino acid residue at position 233 may be mutated, e.g., to Pro, the amino acid residue at position 234 may be mutated, e.g., to Val, and/or the amino acid residue at position 235 may be mutated, e.g., to Ala. The amino acid positions are numbered according to the EU numbering or IMGT® numbering scheme.

In some embodiments, where the antibody is of the IgG4 subclass, it may comprise the mutation S228P, i.e. having a proline in position 228, where the amino acid position is numbered according to the EU numbering or IMGT® numbering scheme. This mutation is known to reduce undesired Fab arm exchange.

In another embodiment, a fusion antibody may be made that comprises all or a fragment of an anti-Gal3 antibody of the invention linked to another polypeptide. In certain embodiments, only the variable domains of the anti-Gal3 antibody are linked to the polypeptide. In certain embodiments, the VH domain of an anti-Gal3 antibody is linked to a first polypeptide, while the VL domain of an anti-Gal3 antibody is linked to a second polypeptide that associates with the first polypeptide in a manner such that the VH and VL domains can interact with one another to form an antigen-binding site. In another preferred embodiment, the VH domain is separated from the VL domain by a linker such that the VH and VL domains can interact with one another (e.g., single-chain antibodies). The VH-linker-VL antibody is then linked to the polypeptide of interest. In addition, fusion antibodies can be created in which two (or more) single-chain antibodies are linked to one another. This is useful if one wants to create a divalent or polyvalent antibody on a single polypeptide chain, or if one wants to create a bi-specific antibody.

In other embodiments, other modified antibodies may be prepared using anti-Gal3 antibody-encoding nucleic acid molecules. For instance, “kappa bodies” (III et al., Protein Eng. 10:949-57 (1997)), “minibodies” (Martin et al., EMBO J. 13:5303-9 (1994)), “diabodies” (Holliger et al., Proc. Natl. Acad. Sci. USA 90:6444-6448 (1993)), or “Janusins” (Traunecker et al., EMBO J. 10:3655-3659 (1991) and Traunecker et al., Int. J. Cancer (Suppl.) 7:51-52 (1992)) may be prepared using standard molecular biological techniques following the teachings of the specification.

An anti-Gal3 antibody or antigen-binding fragment of the invention can be derivatized or linked to another molecule (e.g., another peptide or protein). In general, the antibodies or fragments thereof are derivatized such that Gal3 binding is not affected adversely by the derivatization or labeling. Accordingly, the antibodies and antibody fragments of the invention are intended to include both intact and modified forms of the human anti-Gal3 antibodies described herein. For example, an antibody or antibody fragment of the invention can be functionally linked (by chemical coupling, genetic fusion, noncovalent association or otherwise) to one or more other molecular entities, such as another antibody (e.g., a bi-specific antibody or a diabody), a detection agent, a pharmaceutical agent, and/or a protein or peptide that can mediate association of the antibody or antibody fragment with another molecule (such as a streptavidin core region or a polyhistidine tag).

One type of derivatized antibody is produced by crosslinking two or more antibodies (of the same type or of different types, e.g., to create bi-specific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). An anti-Gal3 antibody or antigen-binding fragment thereof can also be derivatized with a chemical group such as polyethylene glycol (PEG), a methyl or ethyl group, or a carbohydrate group. These groups may be useful to improve the biological characteristics of the antibody, e.g., to increase serum half-life.

An antibody according to the present invention may also be labeled. As used herein, the terms “label” or “labeled” refer to incorporation of another molecule in the antibody. In one embodiment, the label is a detectable marker, e.g., incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). In another embodiment, the label or marker can be therapeutic, e.g., a drug conjugate or toxin. Various methods of labeling polypeptides and glycoproteins are known in the art and may be used. Examples of labels for polypeptides include, but are not limited to, the following: radioisotopes or radionuclides (e.g., 3H, 14C, 15N, 35S, 90Y, 99Tc, 111In, 125I, 131I), fluorescent labels (e.g., FITC, rhodamine, lanthanide phosphors), enzymatic labels (e.g., horseradish peroxidase, β galactosidase, luciferase, alkaline phosphatase), chemiluminescent markers, biotinyl groups, predetermined polypeptide epitopes recognized by a secondary reporter (e.g., leucine zipper pair sequences, binding sites for secondary antibodies, metal binding domains, epitope tags), magnetic agents, such as gadolinium chelates, toxins such as pertussis toxin, taxol, cytochalasin B, gramicidin D, ethidium bromide, emetine, mitomycin, etoposide, tenoposide, vincristine, vinblastine, colchicine, doxorubicin, daunorubicin, dihydroxy anthracin dione, mitoxantrone, mithramycin, actinomycin D, 1 dehydrotestosterone, glucocorticoids, procaine, tetracaine, lidocaine, propranolol, and puromycin and analogs or homologs thereof. In some embodiments, labels are attached by spacer arms of various lengths to reduce potential steric hindrance.

In certain embodiments, the antibodies of the invention may be present in a neutral form (including zwitter ionic forms) or as a positively or negatively-charged species. In some embodiments, the antibodies may be complexed with a counterion to form a pharmaceutically acceptable salt.

The term “pharmaceutically acceptable salt” refers to a complex comprising one or more antibodies and one or more counterions, wherein the counterions are derived from pharmaceutically acceptable inorganic and organic acids and bases.

### Bi-Specific Binding Molecules

In a further aspect, the invention provides a bi-specific binding molecule having the binding specificity (e.g., comprising the antigen-binding fragments) of an anti-Gal3 antibody or antigen-binding fragment thereof described herein and the binding specificity of another anti-Gal3 antibody or antigen-binding fragment thereof (e.g., another anti-Gal3 antibody described herein) or an antibody that targets a same or different protein, such as another immune checkpoint protein, a cancer antigen, or another cell surface molecule whose activity mediates a disease condition such as cancer. Such bi-specific binding molecules are known in the art, and examples of different types of bi-specific binding molecules are given elsewhere herein.

### Nucleic Acid Molecules and Vectors

The present invention also provides nucleic acid molecules and sequences encoding anti-Gal3 antibodies or antigen-binding fragments thereof described herein. In some embodiments, different nucleic acid molecules encode the heavy chain or a VH sequence and encode a light chain amino acid sequences or a VL sequence of the anti-Gal3 antibody or an antigen-binding fragment thereof. In some embodiments, a combination of isolated nucleic acid molecules encodes an anti-Gal3 antibody or an antigen-binding fragment thereof, in particular a first isolated nucleic acid molecule comprising or consisting of a sequence encoding a heavy chain sequence or a VH sequence, and a second isolated nucleic acid molecule comprising or consisting of a sequence encoding a light chain sequence or a VL sequence. In other embodiments, the same nucleic acid molecule encodes the heavy chain or the VH sequence and the light chain or the VL sequence of the anti-Gal3 antibody or an antigen-binding fragment thereof.

A reference to a nucleotide sequence encompasses its complement unless otherwise specified. Thus, a reference to a nucleic acid having a particular sequence should be understood to encompass its complementary strand, with its complementary sequence. The term “polynucleotide” as referred to herein means a polymeric form of nucleotides of at least 10 bases in length, either ribonucleotides or deoxynucleotides or a modified form of either type of nucleotide. The term includes single- and double-stranded forms.

The invention also provides nucleotide sequences that are at least 70%, 75%, 80%, 85%, 90%, 95%, 97%, 98% or 99% identical to one or more nucleotide sequences recited herein, e.g., to a nucleotide sequence selected from the group consisting of SEQ ID NOs: 61-80, or to a nucleotide sequence encoding an amino acid sequence selected from the group consisting of SEQ ID NOs: 38-57. The term “percent sequence identity” in the context of nucleic acid sequences refers to the residues in two sequences that are the same when aligned for maximum correspondence. The length of sequence identity comparison may be over a stretch of at least about nine nucleotides, usually at least about 18 nucleotides, more usually at least about 24 nucleotides, typically at least about 28 nucleotides, more typically at least about 32 nucleotides, and preferably at least about 36, 48 or more nucleotides. There are a number of different algorithms known in the art which can be used to measure nucleotide sequence identity. For instance, polynucleotide sequences can be compared using FASTA, Gap or Bestfit, which are programs in Wisconsin Package Version 10.0, Genetics Computer Group (GCG), Madison, Wisconsin. FASTA, which includes, e.g., the programs FASTA2 and FASTA3, provides alignments and percent sequence identity of the regions of the best overlap between the query and search sequences (see, e.g., Pearson, Methods Enzymol. 183:63-98 (1990); Pearson, Methods Mol. Biol. 132:185-219 (2000); Pearson, Methods Enzymol. 266:227-258 (1996); and Pearson, J. Mol. Biol. 276:71-84 (1998)). Unless otherwise specified, default parameters for a particular program or algorithm are used. For instance, percent sequence identity between nucleic acid sequences can be determined using FASTA with its default parameters (a word size of 6 and the NOPAM factor for the scoring matrix) or using Gap with its default parameters as provided in GCG Version 6.1.

In one aspect, the invention provides a nucleic acid molecule comprising a nucleotide sequence selected from the group consisting of SEQ ID NOs: 61-80. In certain embodiments, the nucleic acid molecule comprises the nucleotide sequences of SEQ ID NOs: 61 and 62, SEQ ID NOs: 63 and 64, SEQ ID NOs: 65 and 66, SEQ ID NOs: 67 and 68, SEQ ID NOs: 69 and 70, SEQ ID NOs: 71 and 72, SEQ ID NOs: 73 and 74, SEQ ID NOs: 75 and 76, SEQ ID NOs: 77 and 78 or SEQ ID NOs: 79 and 80.

In any of the above embodiments, the nucleic acid molecules may be isolated.

In a further aspect, the present invention provides a vector suitable for expressing one of the chains of an antibody or antigen-binding fragment thereof as described herein. The term “vector”, as used herein, means a nucleic acid molecule capable of transporting another nucleic acid to which it has been linked. In some embodiments, the vector is a plasmid, i.e., a circular double stranded piece of DNA into which additional DNA segments may be ligated. In some embodiments, the vector is a viral vector, wherein additional DNA segments may be ligated into the viral genome. In some embodiments, the vectors are capable of autonomous replication in a host cell into which they are introduced (e.g., bacterial vectors having a bacterial origin of replication and episomal mammalian vectors). In other embodiments, the vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”).

The invention provides vectors comprising nucleic acid molecules that encode the heavy chain or VH of an anti-Gal3 antibody of the invention or an antigen-binding fragment thereof, the light chain or VL of an anti-Gal3 antibody of the invention or an antigen-binding fragment thereof, or both the heavy and light chains or VH and VL of an anti-Gal3 antibody of the invention or an antigen-binding fragment thereof. The invention further provides vectors comprising nucleic acid molecules encoding fusion proteins, modified antibodies, antibody fragments, and probes thereof.

A nucleic acid molecule encoding the heavy and/or light chain of an anti-Gal3 antibody or antigen-binding fragment thereof of the invention can be isolated from any source that produces such an antibody or fragment. In various embodiments, the nucleic acid molecules are isolated from B cells that express an anti-Gal3 antibody isolated from an animal immunized with a human Gal3 antigen, or from an immortalized cell produced from such a B cell. Methods of isolating nucleic acids encoding an antibody are well-known in the art. mRNA may be isolated and used to produce cDNA for use in polymerase chain reaction (PCR) or cDNA cloning of antibody genes. In certain embodiments, a nucleic acid molecule of the invention can be synthesized rather than isolated.

In some embodiments, a nucleic acid molecule of the invention can comprise a nucleotide sequence encoding a VH domain from an anti-Gal3 antibody or antigen-binding fragment of the invention joined in-frame to a nucleotide sequence encoding a heavy chain constant region from any source. Similarly, a nucleic acid molecule of the invention can comprise a nucleotide sequence encoding a VL domain from an anti-Gal3 antibody or antigen-binding fragment of the invention joined in-frame to a nucleotide sequence encoding a light chain constant region from any source.

In a further aspect of the invention, nucleic acid molecules encoding the variable domain of the heavy (VH) and/or light (VL) chains may be “converted” to full-length antibody genes. In one embodiment, nucleic acid molecules encoding the VH or VL domains are converted to full-length antibody genes by insertion into an expression vector already encoding heavy chain constant (CH) or light chain constant (CL) regions, respectively, such that the VH segment is operatively linked to the CH segment(s) within the vector, and/or the VL segment is operatively linked to the CL segment within the vector. In another embodiment, nucleic acid molecules encoding the VH and/or VL domains are converted into full-length antibody genes by linking, e.g., ligating, a nucleic acid molecule encoding a VH and/or VL domains to a nucleic acid molecule encoding a CH and/or CL region using standard molecular biological techniques. Nucleic acid molecules encoding the full-length heavy and/or light chains may then be expressed from a cell into which they have been introduced and the anti-Gal3 antibody isolated.

The nucleic acid molecules may be used to recombinantly express large quantities of anti-Gal3 antibodies. The nucleic acid molecules also may be used to produce chimeric antibodies, bi-specific antibodies, single chain antibodies, immunoadhesins, diabodies, mutated antibodies and antibody derivatives, as described herein.

In another embodiment, a nucleic acid molecule of the invention is used as a probe or PCR primer for a specific antibody sequence. For instance, the nucleic acid can be used as a probe in diagnostic methods or as a PCR primer to amplify regions of DNA that could be used, inter alia, to isolate additional nucleic acid molecules encoding variable domains of anti-Gal3 antibodies. In some embodiments, the nucleic acid molecules are oligonucleotides. In some embodiments, the oligonucleotides are from highly variable domains of the heavy and light chains of the antibody of interest. In some embodiments, the oligonucleotides encode all or a part of one or more of the CDRs of the anti-Gal3 antibodies or antigen-binding fragments thereof of the invention as described herein.

In another embodiment, the nucleic acid molecules and vectors may be used to make mutated anti-Gal3 antibodies or antigen-binding fragments thereof. The antibodies may be mutated in the variable domains of the heavy and/or light chains, e.g., to alter a binding property of the antibody. For example, a mutation may be made in one or more of the CDRs or increase or decrease the KD of the anti-Gal3 antibody or antigen-binding fragment thereof or to alter the binding specificity of the antibody or antigen-binding fragment thereof. In another embodiment, one or more mutations are made at an amino acid residue that is known to be changed compared to the germline in a monoclonal antibody of the invention. The mutations may be made in a CDR or framework region of a variable domain, or in a constant region. In a preferred embodiment, the mutations are made in a variable domain. In some embodiments, one or more mutations are made at an amino acid residue that is known to be changed compared to the germline in a CDR or framework region of a variable domain of an antibody or antigen-binding fragment thereof of the invention.

In another embodiment, the framework region(s) are mutated so that the resulting framework region(s) have the amino acid sequence of the corresponding germline gene. A mutation may be made in a framework region or constant region to increase the half-life of the anti-Gal3 antibody. See, e.g., PCT Publication WO 00/09560. A mutation in a framework region or constant region also can be made to alter the immunogenicity of the antibody, and/or to provide a site for covalent or non-covalent binding to another molecule. According to the invention, a single antibody may have mutations in any one or more of the CDRs or framework regions of the variable domain or in the constant region.

In some embodiments, the anti-Gal3 antibodies of the invention or antigen-binding fragments thereof are expressed by inserting DNAs encoding partial or full-length light and heavy chains, obtained as described above, into expression vectors such that the genes are operatively linked to necessary expression control sequences such as transcriptional and translational control sequences. Expression vectors include plasmids, retroviruses, adenoviruses, adeno-associated viruses (AAV), plant viruses such as cauliflower mosaic virus, tobacco mosaic virus, cosmids, YACs, EBV derived episomes, and the like. The antibody coding sequence may be ligated into a vector such that transcriptional and translational control sequences within the vector serve their intended function of regulating the transcription and translation of the antibody coding sequence. The expression vector and expression control sequences may be chosen to be compatible with the expression host cell used. The antibody light chain coding sequence and the antibody heavy chain coding sequence can be inserted into separate vectors, and may be operatively linked to the same or different expression control sequences (e.g., promoters). In one embodiment, both coding sequences are inserted into the same expression vector and may be operatively linked to the same expression control sequences (e.g., a common promoter), to separate identical expression control sequences (e.g., promoters), or to different expression control sequences (e.g., promoters). The antibody coding sequences may be inserted into the expression vector by standard methods (e.g., ligation of complementary restriction sites on the antibody gene fragment and vector, or blunt end ligation if no restriction sites are present).

A convenient vector is one that encodes a functionally complete human CH or CL immunoglobulin sequence, with appropriate restriction sites engineered so that any VH or VL sequence can easily be inserted and expressed, as described above. The HC- and LC-encoding genes in such vectors may contain intron sequences that will result in enhanced overall antibody protein yields by stabilizing the related mRNA. The intron sequences are flanked by splice donor and splice acceptor sites, which determine where RNA splicing will occur. Location of intron sequences can be either in variable or constant regions of the antibody chains, or in both variable and constant regions when multiple introns are used. Polyadenylation and transcription termination may occur at native chromosomal sites downstream of the coding regions. The recombinant expression vector also can encode a signal peptide that facilitates secretion of the antibody chain from a host cell. The antibody chain gene may be cloned into the vector such that the signal peptide is linked in-frame to the amino terminus of the immunoglobulin chain. The signal peptide can be an immunoglobulin signal peptide or a heterologous signal peptide (i.e., a signal peptide from a non immunoglobulin protein).

In addition to the antibody chain genes, the recombinant expression vectors of the invention may carry regulatory sequences that control the expression of the antibody chain genes in a host cell. It will be appreciated by those skilled in the art that the design of the expression vector, including the selection of regulatory sequences, may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from retroviral LTRs, cytomegalovirus (CMV) (such as the CMV promoter/enhancer), Simian Virus 40 (SV40) (such as the SV40 promoter/enhancer), adenovirus, (e.g., the adenovirus major late promoter (AdMLP)), polyoma and strong mammalian promoters such as native immunoglobulin and actin promoters. For further description of viral regulatory elements, and sequences thereof, see e.g., U.S. Pat. Nos. 5,168,062, 4,510,245 and 4,968,615. Methods for expressing antibodies in plants, including a description of promoters and vectors, as well as transformation of plants, are known in the art. See, e.g., U.S. Pat. No. 6,517,529. Methods of expressing polypeptides in bacterial cells or fungal cells, e.g., yeast cells, are also well known in the art.

In addition to the antibody chain genes and regulatory sequences, the recombinant expression vectors of the invention may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin or methotrexate, on a host cell into which the vector has been introduced. For example, selectable marker genes include the dihydrofolate reductase (DHFR) gene (for use in dhfr-host cells with methotrexate selection/amplification), the neo gene (for G418 selection), and the glutamate synthetase gene.

The term “expression control sequence” as used herein means polynucleotide sequences that are necessary to effect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences.

### Host Cells and Methods of Antibody and Antibody Composition Production

An additional aspect of the invention relates to methods for producing the antibody compositions and antibodies and antigen-binding fragments thereof of the invention. One embodiment of this aspect of the invention relates to a method for producing an antibody as defined herein, comprising providing a recombinant host cell capable of expressing the antibody, cultivating said host cell under conditions suitable for expression of the antibody, and isolating the resulting antibody. Antibodies produced by such expression in such recombinant host cells are referred to herein as “recombinant antibodies.” The invention also provides progeny cells of such host cells, and antibodies produced by same.

The term “recombinant host cell” (or simply “host cell”), as used herein, means a cell into which a recombinant expression vector has been introduced. The invention provides host cells that may comprise, e.g., a vector according to the invention described above. The invention also provides host cells that comprise, e.g., a nucleotide sequence encoding the heavy chain or an antigen-binding fragment thereof, a nucleotide sequence encoding the light chain or an antigen-binding fragment thereof, or both, of an anti-Gal3 antibody or antigen-binding fragment thereof of the invention. It should be understood that “recombinant host cell” and “host cell” mean not only the particular subject cell but also the progeny of such a cell. Because certain modifications may occur in succeeding generations due to either mutation or environmental influences, such progeny may not, in fact, be identical to the parent cell, but are still included within the scope of the term “host cell” as used herein.

Nucleic acid molecules encoding anti-Gal3 antibodies and vectors comprising these nucleic acid molecules can be used for transfection of a suitable mammalian, plant, bacterial or yeast host cell. Transformation can be by any known method for introducing polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, encapsulation of the polynucleotide(s) in liposomes, and direct microinjection of the DNA into nuclei. In addition, nucleic acid molecules may be introduced into mammalian cells by viral vectors. Methods of transforming cells are well known in the art. See, e.g., U.S. Pat. Nos. 4,399,216, 4,912,040, 4,740,461, and 4,959,455. Methods of transforming plant cells are well known in the art, including, e.g., Agrobacterium-mediated transformation, biolistic transformation, direct injection, electroporation and viral transformation. Methods of transforming bacterial and yeast cells are also well known in the art.

Mammalian cell lines available as hosts for expression are well known in the art and include many immortalized cell lines available from the American Type Culture Collection (ATCC). These include, inter alia, Chinese hamster ovary (CHO) cells, NS0 cells, SP2 cells, HEK-293T cells, 293 Freestyle cells (Invitrogen), NIH-3T3 cells, HeLa cells, baby hamster kidney (BHK) cells, African green monkey kidney cells (COS), human hepatocellular carcinoma cells (e.g., Hep G2), A549 cells, and a number of other cell lines. Cell lines of particular preference are selected by determining which cell lines have high expression levels. Other cell lines that may be used are insect cell lines, such as Sf9 or Sf21 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods. Plant host cells include, e.g., Nicotiana, Arabidopsis, duckweed, corn, wheat, potato, etc. Bacterial host cells include E. coli and Streptomyces species. Yeast host cells include Schizosaccharomyces pombe, Saccharomyces cerevisiae and Pichia pastoris.

Further, expression of antibodies of the invention or antigen-binding fragments thereof from production cell lines can be enhanced using a number of known techniques. For example, the glutamine synthetase gene expression system (the GS system) is a common approach for enhancing expression under certain conditions. The GS system is discussed in whole or part in connection with EP Patents 0 216 846, 0 256 055, 0 323 997 and 0 338 841.

It is likely that antibodies expressed by different cell lines or in transgenic animals will have different glycosylation patterns from each other. However, all antibodies encoded by the nucleic acid molecules provided herein, or comprising the amino acid sequences provided herein are part of the instant invention, regardless of the glycosylation state of the antibodies, and more generally, regardless of the presence or absence of post-translational modification(s).

### Pharmaceutical Compositions

Another aspect of the invention is a pharmaceutical composition comprising as an active ingredient (or as the sole active ingredient) an anti-Gal3 antibody or antigen-binding fragment thereof, bi-specific binding molecule, or antibody composition of the invention. The pharmaceutical composition may comprise any anti-Gal3 antibody or antigen-binding fragment thereof, bi-specific binding molecule, or antibody composition as described herein. In some embodiments, the pharmaceutical compositions are intended for amelioration, prevention, and/or treatment of a Gal3-related disorder or disease. As used herein, a Gal3-related disorder or Gal3-related disease refers to a condition that improves, or slows down in its progression, by modulation of Gal3 expression, interaction and/or activity. In certain embodiments, the compositions are intended for amelioration, prevention, and/or treatment of fibrotic diseases, inflammatory diseases, autoimmune diseases, immune-mediated disorders, neurodegenerative diseases, metabolic diseases, infectious diseases as well as several cancers.

Generally, the antibodies, antigen-binding fragments, and bi-specific binding molecules of the invention are suitable to be administered as a formulation in association with one or more pharmaceutically acceptable excipient(s), e.g., as described below.

Pharmaceutical compositions of the invention will comprise one or more anti-Gal3 antibodies or antigen-binding fragments thereof, or bi-specific binding molecules of the invention, e.g., one or two anti-Gal3 antibodies or antigen-binding fragments thereof, or bi-specific binding molecules. In one embodiment, the composition comprises a single anti-Gal3 antibody or antigen-binding fragment thereof of the invention. In one embodiment, the composition comprises multiple anti-Gal3 antibodies of the invention or antigen-binding fragments thereof.

In another embodiment, the pharmaceutical composition may comprise at least one anti-Gal3 antibody or antigen-binding fragment thereof, e.g., one anti-Gal3 antibody or antigen-binding fragment thereof, and one or more additional antibodies that target one or more relevant cell surface receptors, e.g., one or more cancer-relevant receptors.

The term “excipient” is used herein to describe any ingredient other than the compound(s) of the invention. The choice of excipient(s) will to a large extent depend on factors such as the particular mode of administration, the effect of the excipient on solubility and stability, and the nature of the dosage form. As used herein, “pharmaceutically acceptable excipient” includes any and all solvents, dispersion media, coatings, antibacterial and antifungal agents, isotonic and absorption delaying agents, and the like that are physiologically compatible. Some examples of pharmaceutically acceptable excipients are water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Additional examples of pharmaceutically acceptable substances are wetting agents or minor amounts of auxiliary substances such as wetting or emulsifying agents, preservatives or buffers, which enhance the shelf life or effectiveness of the antibody.

Pharmaceutical compositions of the present invention and methods for their preparation will be readily apparent to those skilled in the art. Such compositions and methods for their preparation may be found, for example, in Remington's Pharmaceutical Sciences, 19th Edition (Mack Publishing Company, 1995). Pharmaceutical compositions are preferably manufactured under GMP (good manufacturing practices) conditions.

A pharmaceutical composition of the invention may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

Any method for administering peptides, proteins or antibodies accepted in the art may suitably be employed for the antibodies and antigen-binding fragments of the invention.

The pharmaceutical compositions of the invention are typically suitable for parenteral administration. As used herein, “parenteral administration” of a pharmaceutical composition includes any route of administration characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal, intravenous, intraarterial, intrathecal, intraventricular, intraurethral, intracranial, intratumoral, and intrasynovial injection or infusions; and kidney dialytic infusion techniques. Regional perfusion is also contemplated. Particular embodiments include the intravenous and the subcutaneous routes.

Formulations of a pharmaceutical composition suitable for parenteral administration typically comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi dose containers containing a preservative. Formulations for parenteral administration include, but are not limited to, suspensions, solutions, emulsions in oily or aqueous vehicles, pastes, and the like. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen free water) prior to parenteral administration of the reconstituted composition. Parenteral formulations also include aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water. Exemplary parenteral administration forms include solutions or suspensions in sterile aqueous solutions, for example, aqueous propylene glycol or dextrose solutions. Such dosage forms can be suitably buffered, if desired. Other parentally-administrable formulations which are useful include those which comprise the active ingredient in microcrystalline form, or in a liposomal preparation. Formulations for parenteral administration may be formulated to be immediate and/or modified release. Modified release formulations include delayed-, sustained-, pulsed-, controlled-, targeted and programmed release.

For example, in one aspect, sterile injectable solutions can be prepared by incorporating the anti-Gal3 antibody, antigen-binding fragment thereof, bi-specific binding molecule, or antibody composition in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin, and/or by using modified-release coatings (e.g., slow-release coatings).

The antibodies of the invention can also be administered intranasally or by inhalation, typically in the form of a dry powder (either alone, as a mixture, or as a mixed component particle, for example, mixed with a suitable pharmaceutically acceptable excipient) from a dry powder inhaler, as an aerosol spray from a pressurised container, pump, spray, atomiser (preferably an atomiser using electrohydrodynamics to produce a fine mist), or nebuliser, with or without the use of a suitable propellant, or as nasal drops.

The pressurised container, pump, spray, atomizer, or nebuliser generally contains a solution or suspension of an antibody of the invention comprising, for example, a suitable agent for dispersing, solubilising, or extending release of the active, a propellant(s) as solvent.

Prior to use in a dry powder or suspension formulation, the drug product is generally micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules, blisters and cartridges for use in an inhaler or insufflator may be formulated to contain a powder mix of the compound of the invention, a suitable powder base and a performance modifier.

A suitable solution formulation for use in an atomiser using electrohydrodynamics to produce a fine mist may contain a suitable dose of the antibody of the invention per actuation and the actuation volume may for example vary from 1 μL to 100 μL.

Formulations for inhaled/intranasal administration may be formulated to be immediate and/or modified release. Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff” of an antibody of the invention. The overall daily dose will typically be administered in a single dose or, more usually, as divided doses throughout the day.

The antibodies and antibody fragments of the invention may also be formulated for an oral route administration. Oral administration may involve swallowing, so that the compound enters the gastrointestinal tract, and/or buccal, lingual, or sublingual administration by which the compound enters the blood stream directly from the mouth.

Formulations suitable for oral administration include solid, semi-solid and liquid systems such as tablets; soft or hard capsules containing multi- or nano-particulates, liquids, or powders; lozenges (including liquid-filled); chews; gels; fast dispersing dosage forms; films; ovules; sprays; and buccal/mucoadhesive patches.

Liquid formulations include suspensions, solutions, syrups and elixirs. Such formulations may be employed as fillers in soft or hard capsules (made, for example, from gelatin or hydroxypropylmethylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

### Therapeutic Uses of Antibodies and Compositions of the Invention

In one aspect, the anti-Gal3 antibodies and antigen-binding fragments thereof, anti-Gal3 compositions, and bi-specific binding molecules of the invention are used to inhibit Gal3 expression, interaction and/or activity in a human in need thereof. Anti-Gal3 antibody or antigen-binding fragment thereof of the present invention can be administered alone or in combination with other therapeutic agents (sequentially or concurrently).

In certain embodiments, the antibody or antigen-binding fragment thereof, composition, or bi-specific binding molecule is for use in the amelioration, prevention and/or treatment of a number of fibrotic diseases, inflammatory diseases, autoimmune diseases, immune-mediated disorders, neurodegenerative diseases, metabolic diseases, infectious diseases and several cancers.

The term “fibrosis” refers to the medical condition wherein tissues or organs harden or scar as a result of unregulated production of extracellular matrix, such as collagen proteins. Fibrosis has been associated with chronic inflammation, where immune cells such as macrophages signal fibroblasts to express extracellular matrix proteins in response. This signaling is achieved through pathways such as the IL-6 pathway, IL-5 pathway, although there are other pro-fibrotic pathways as well. Fibrosis includes but is not limited to liver fibrosis, bridging fibrosis, cirrhosis, kidney fibrosis, pulmonary fibrosis, asbestosis, silicosis, coal workers pneumoconiosis and diffuse dust fibrosis, chemotherapeutic agent-induced pulmonary fibrosis, idiopathic pulmonary fibrosis, cystic fibrosis, polycystic kidney disease, cardiovascular fibrosis, Chagas disease, arterial fibrosis, venous thrombosis, cardiac fibrosis, pulmonary arterial fibrosis, arthro fibrosis, Dupuytren's contracture, keloids, mediastinal fibrosis, myelofibrosis, Peyronie's disease, nephrogenic systemic fibrosis, tubulointerstitial fibrosis, glomerulosclerosis progressive massive fibrosis, aristolochic-acid and Balkan endemic nephropathies, progressive massive fibrosis, retroperitoneal fibrosis, or systemic sclerosis.

In some embodiments, cancers prevented or treated by the anti-Gal3 antibodies, antigen-binding fragments, bi-specific binding molecules, and/or antibody compositions of the invention may include cancer, such as carcinomas, sarcomas, leukemias and lymphomas, such as T-cell lymphomas; metastasizing cancers. It can further include melanoma (e.g., advanced or metastatic melanoma), non-small cell lung cancer, head and neck squamous cell cancer, renal cell carcinoma, Hodgkin's lymphoma, non-Hodgkin's lymphoma, glioblastoma, glioma, squamous cell lung cancer, small-cell lung cancer, hepatocellular carcinoma, bladder cancer, upper urinary tract cancer, esophageal cancer, gastroesophageal junction cancer, gastric cancer, liver cancer, colon cancer, colorectal carcinoma, multiple myeloma, sarcomas, acute myeloid leukemia, chronic myeloid leukemia, myelodysplastic syndrome, nasopharyngeal cancer, chronic lymphocytic leukemia, acute lymphoblastic leukemia, small lymphocytic lymphoma, ovarian cancer, gastrointestinal cancer, primary peritoneal cancer, fallopian tube cancer, urothelial cancer, HTLV-associated T-cell leukemia/lymphoma, prostate cancer, genitourinary cancer, meningioma, adrenocortical cancer, gliosarcoma, fibrosarcoma, kidney cancer, breast cancer, pancreatic cancer, endometrial cancer, skin basal cell cancer, cancer of the appendix, biliary tract cancer, salivary gland cancer, advanced Merkel cell cancer, diffuse large B cell lymphoma, follicular lymphoma, mesothelioma, or solid tumors. The cancer may be, e.g., at an early, intermediate, late, or metastatic stage.

The terms “cancer”, “tumor”, and “carcinoma” are used interchangeably herein to refer to cells which exhibit relatively autonomous growth, so that they exhibit an aberrant growth phenotype characterized by a significant loss of control of cell proliferation. In general, cells of interest for detection or treatment in the present application include precancerous (e.g., benign), malignant, pre-metastatic, metastatic, and non-metastatic cells. Detection of cancerous cells is of particular interest. The term “normal” as used in the context of “normal cell,” is meant to refer to a cell of an untransformed phenotype or exhibiting a morphology of a non-transformed cell of the tissue type being examined. “Cancerous phenotype” generally refers to any of a variety of biological phenomena that are characteristic of a cancerous cell, which phenomena can vary with the type of cancer. The cancerous phenotype is generally identified by abnormalities in, for example, cell growth or proliferation (e.g., uncontrolled growth or proliferation), regulation of the cell cycle, cell mobility, cell-cell interaction, or metastasis, etc.

In one aspect, the anti-Gal3 antibodies and antigen-binding fragments thereof, antibody compositions, or bi-specific binding molecules of the invention may be used to prevent or treat an inflammatory disease or autoimmune disease or autoimmune disorder or immune-mediated disease or immune-mediated disorder such as psoriasis, rheumatoid arthritis, juvenile idiopathic arthritis, osteoarthritis, ulcerative colitis, ankylosing spondylitis, autoimmune uveitis, primary biliary cholangitis, autoimmune and inflammatory nephropathies, sepsis, type 1 diabetes, inflammatory myopathies, myocarditis, pericarditis, allergy, asthma, gastric ulcer, gastritis, systemic lupus erythematosus, sjögren's syndrome, inflammatory bowel disease (IBD), Crohn's disease, atopic dermatitis, systemic sclerosis or cutaneous sclerosis.

In one aspect, the anti-Gal3 antibodies and antigen-binding fragments thereof, antibody compositions, or bi-specific binding molecules of the invention may be used to prevent or treat metabolic disorders, heart disease, heart failure, myocardial infarction, pathological angiogenesis, atherosclerosis, hypertension, pulmonary hypertension, venous thrombosis, metabolic diseases such as diabetes, type 2 diabetes, type 1 diabetes, insulin resistance, obesity, diastolic heart failure, asthma and other interstitial lung diseases, including Hermansky-Pudlak syndrome, mesothelioma, chronic obstructive pulmonary diseases, endometriosis, liver disorders, such as non-alcoholic steatohepatitis.

In one aspect, the anti-Gal3 antibodies and antigen-binding fragments thereof, antibody compositions, or bi-specific binding molecules of the invention may be used to prevent or treat a neurodegenerative disease or a disease affecting the central and/or peripheral nervous system of a patient. The anti-Gal3 antibodies and binding fragments thereof disclosed herein disrupt the interaction between Gal3 and proteins associated with neurodegenerative diseases. In some embodiments, the proteins associated with neurodegenerative diseases cause disease due to misfolding or aggregation of the proteins in a subject. These diseases may involve the death of neurons or other cell types associated with the nervous system. Non-limiting examples of neurological disorders include encephalitis, Alzheimer's disease, Parkinson's disease, Huntington's disease, traumatic brain injury, spinal injury, multiple sclerosis, amyotrophic lateral sclerosis, olfactory dysfunction, aphasia, Bell's palsy, transmissible spongiform encephalopathy, Creutzfeldt-Jakob disease, fatal familial insomnia, epilepsy, seizures, neurodevelopment, Tourette's syndrome, neuroinfectious disorders, meningitis, encephalitis, bovine spongiform encephalopathy, West Nile virus encephalitis, Neuro-AIDS, fragile X syndrome, Guillain-Barre syndrome, metastases to the brain, or brain cancer, or otherwise known by a person skilled in the art.

In one aspect, the anti-Gal3 antibodies and antigen-binding fragments thereof, antibody compositions, or bi-specific binding molecules of the invention may be used to prevent or treat infectious diseases, such as viral or parasitic or pathogen infections and their consequences, including those induced by coronavirus, papilloma virus, influenza virus, encephalomyocarditis virus, Trypanosoma cruzi.

“Treat”, “treating” and “treatment” refer to a method of alleviating or abrogating a biological disease or disorder and/or at least one of its attendant symptoms. As used herein, to “alleviate” a disease, disorder or condition means reducing the severity and/or occurrence frequency of the symptoms of the disease, disorder, or condition. Further, references herein to “treatment” include references to curative, palliative and prophylactic treatment.

“Therapeutically effective amount” refers to the amount of the therapeutic agent being administered that will relieve to some extent one or more of the symptoms of the disease or disorder being treated. A therapeutically effective amount of an anti-cancer therapeutic may, for example, result in tumor shrinkage, increased survival, elimination of cancer cells, decreased disease progression, reversal of metastasis, or other clinical endpoints desired by healthcare professionals.

In some embodiments, also described herein are methods of monitoring the progression of a tissue fibrosis by monitoring one or more fibrosis biomarkers, that comprises or consists of a-smooth muscle actin, fibronectin, collagen, elastin, laminin, hyaluronic acid, or proteoglycans, or any combination thereof.

In some embodiments, the anti-Gal3 antibody or antigen-binding fragment thereof results in reduced accumulation of extracellular matrix proteins in the tissue. In some embodiments, the anti-Gal3 antibody or antigen-binding fragment thereof is for use in the amelioration, prevention, and/or treatment of a disease, wherein the disease is a fibrotic disease or fibrosis. In some embodiments, the anti-Gal3 antibody or antigen-binding fragment thereof is for use in the amelioration, prevention, and/or treatment of a disease results in reduced accumulation of extracellular matrix proteins in a tissue. In some embodiments, the extracellular matrix is comprised or consists of agrin, nidogen, cadherins, clathrin, collagen, defensin, elastin, entactin, fibrillin, fibronectin, keratin, laminin, microtubule-actin cross-linking factor 1, SPARC-like protein, nesprin, fibrous sheath-interacting protein, myomesin, nebulin, plakophilin, integrin, talins, exportins, transportin, tenascin, perlecan, sortilin-related receptor, tensin, or titin or any combination thereof. In some embodiments, the extracellular matrix proteins comprise or consist of collagen. In some embodiments, the tissue comprises or consists of a collagen-producing cell. In some embodiments, the collagen-producing cell is a fibroblast cell. In some embodiments, the fibroblast cell is activated by a fibrogenic cytokine. In some embodiments, the fibrogenic cytokine is TGF-b, IL-1b, TNF-a, IL-5 or IL-6. In some embodiments, the tissue has an elevated fibrogenic cytokine expression.

The anti-Gal3 antibodies or antigen-binding fragments thereof, antibody compositions, or bi-specific binding molecules of the invention may be administered alone or in combination with one or more other drugs or antibodies (or as any combination thereof). The pharmaceutical compositions, methods and uses of the invention thus also encompass embodiments of combinations (co-administration) with other active agents, as detailed below.

As used herein, the terms “co-administration”, “co-administered” and “in combination with,” referring to the anti-Gal3 antibodies and antigen-binding fragments thereof, antibody compositions, and bi-specific binding molecules of the invention with one or more other therapeutic agents, is intended to mean, and does refer to and include the following:
a) simultaneous administration of such combination of antibody/antigen-binding fragment/antibody composition/bi-specific binding molecule of the invention and therapeutic agent(s) to a patient in need of treatment, when such components are formulated together into a single dosage form which releases said components at substantially the same time to said patient,
b) substantially simultaneous administration of such combination of antibody/antigen-binding fragment/antibody composition/bi-specific binding molecule of the invention and therapeutic agent(s) to a patient in need of treatment, when such components are formulated apart from each other into separate dosage forms which are taken at substantially the same time by said patient, whereupon said components are released at substantially the same time to said patient,
c) sequential administration of such combination of antibody/antigen-binding fragment/antibody composition/bi-specific binding molecule of the invention and therapeutic agent(s) to a patient in need of treatment, when such components are formulated apart from each other into separate dosage forms which are taken at consecutive times by said patient with a significant time interval between each administration, whereupon said components are released at substantially different times to said patient; and
d) sequential administration of such combination of antibody/antigen-binding fragment/antibody composition/bi-specific binding molecule of the invention and therapeutic agent(s) to a patient in need of treatment, when such components are formulated together into a single dosage form which releases said components in a controlled manner whereupon they are concurrently, consecutively, and/or overlappingly released at the same and/or different times to said patient, where each part may be administered by either the same or a different route.

The anti-Gal3 antibodies and antigen-binding fragments thereof, antibody compositions, or bi-specific binding molecules of the invention may be administered without additional therapeutic treatments, i.e., as a stand-alone therapy (monotherapy). Alternatively, treatment with the anti-Gal3 antibodies and antigen-binding fragments thereof, antibody compositions, or bi-specific binding molecules of the invention may include at least one additional further therapeutic treatment (combination therapy). The additional therapeutic treatment may comprise the administration to the patient of a further additional therapeutic agent, e.g., an immune checkpoint modulator, a chemotherapeutic agent, anti-neoplastic agent, anti-angiogenic agent, hormonal therapeutic agent, stem cell-based therapeutic agent, surgery, and/or radiation therapy. The additional therapeutic treatment may comprise PD1/PDL1 blockade therapies and/or a CTLA4 blockade therapy, comprising the administration to the patient of a further additional therapeutic agent. In some embodiments, the PD1/PDL1 blockade therapies comprise the administration to the patient of a therapeutic agent such as pembrolizumab, nivolumab, cemiplimab, spartalizumab, camrelizumab, sintilimab, tislelizumab, toripalimab, AMP-224, AMP-514, atezolizumab, avelumab, durvalumab, KN035, CK-301, AUNP12, CA-170, and/or BMS-986189. In some embodiments, the CTLA4 blockade therapy comprises comprise the administration to the patient of a therapeutic agent such as ipilimumab and/or tremilimumab.

It is also contemplated that an anti-Gal3 antibody or antigen-binding fragment thereof, antibody composition, or bi-specific binding molecule of the invention may be used in adjunctive therapy in connection with a further additional therapeutic agent which is tyrosine kinase inhibitors. These are synthetic, mainly quinazoline-derived, low molecular weight molecules that interact with the intracellular tyrosine kinase domain of receptors and inhibit ligand-induced receptor phosphorylation by competing for the intracellular Mg-ATP binding site.

In certain aspects, the antibodies and antigen-binding fragments, antibody compositions, or bi-specific binding molecules of the invention may be administered in combination with a further additional therapeutic agent which is another inhibitor of the Gal3 pathway, which may target Gal3 or one or more of its ligands. Examples of such inhibitors include other anti-Gal3 antibodies or Gal3-targeting small molecules.

It is understood that the antibodies and antigen-binding fragments thereof, antibody compositions, and bi-specific binding molecules of the invention may be used in a method of amelioration, prevention, and/or treatment as described herein, may be for use in a ameliorating, preventing, and/or treating as described herein, and/or may be for use in the manufacture of a medicament for an amelioration, prevention, and/or treatment as described herein. The invention also provides kits and articles of manufacture comprising the antibodies and antigen-binding fragments thereof, antibody compositions, and bi-specific binding molecules described herein.

### Dose and Route of Administration

The antibodies or antigen-binding fragments thereof, antibody compositions, or bi-specific binding molecules of the invention will be administered in an effective amount for amelioration, prevention, and/or treatment of the condition in question, i.e., at dosages and for periods of time necessary to achieve a desired result. A therapeutically effective amount may vary according to factors such as the particular condition being treated, the age, sex and weight of the patient, and whether the antibodies are being administered as a stand-alone treatment or in combination with one or more additional anti-cancer treatments.

Dosage regimens may be adjusted to provide the optimum desired response. For example, a single bolus may be administered, several divided doses may be administered over time or the dose may be profragmentally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the patients/subjects to be treated; each unit containing a predetermined quantity of active compound calculated to produce the desired therapeutic effect in association with the required pharmaceutical carrier. The specification for the dosage unit forms of the invention are generally dictated by and directly dependent on (a) the unique characteristics of the chemotherapeutic agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen are adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present invention.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the embodied composition. Further, the dosage regimen with the compositions of this invention may be based on a variety of factors, including the type of disease, the age, weight, sex, medical condition of the patient, the severity of the condition, the route of administration, and the particular antibody employed. Thus, the dosage regimen can vary widely, but can be determined routinely using standard methods. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan. Determining appropriate dosages and regimens are well-known in the relevant art and would be understood to be encompassed by the skilled artisan once provided the teachings disclosed herein.

It is contemplated that a suitable dose of an antibody, antigen-binding fragment, antibody composition, or bi-specific binding molecule of the invention will be in the range of 0.1-100 mg/kg, such as about 0.5-50 mg/kg, e.g., about 1-20 mg/kg. The antibody, antigen-binding fragment, antibody composition, or bi-specific binding molecule may for example be administered in a dosage of at least 0.25 mg/kg, e.g., at least 0.5 mg/kg, such as at least 1 mg/kg, e.g., at least 1.5 mg/kg, such as at least 2 mg/kg, e.g., at least 3 mg/kg, such as at least 4 mg/kg, e.g., at least 5 mg/kg; and e.g., up to at most 50 mg/kg, such as up to at the most 30 mg/kg, e.g., up to at the most 20 mg/kg, such as up to at the most 15 mg/kg. Administration will normally be repeated at suitable intervals, e.g., once every week, once every two weeks, once every three weeks, or once every four weeks, and for as long as deemed appropriate by the responsible doctor, who may optionally increase or decrease the dosage as necessary.

An effective amount for tumor therapy may be measured by its ability to stabilize disease progression and/or ameliorate symptoms in a patient, and preferably to reverse disease progression, e.g., by reducing tumor size. The ability of an antibody, antigen-binding fragment, antibody composition, or bi-specific binding molecule of the invention to inhibit cancer may be evaluated by in vitro assays, e.g., as described in the examples, as well as in suitable animal models that are predictive of the efficacy in human tumors. Suitable dosage regimens will be selected in order to provide an optimum therapeutic response in each particular situation, for example, administered as a single bolus or as a continuous infusion, and with possible adjustment of the dosage as indicated by the exigencies of each case.

### Diagnostic Uses and Compositions

The antibodies of the present invention also are useful in diagnostic processes (e.g., in vitro, ex vivo). For example, the antibodies can be used to detect and/or measure the level of Gal3 in a sample from a patient (e.g., a tissue sample, or a body fluid sample such as an inflammatory exudate, blood, serum, bowel fluid, saliva, or urine). Suitable detection and measurement methods include immunological methods such as flow cytometry, enzyme-linked immunosorbent assays (ELISA), chemiluminescence assays, radioimmunoassay, and immunohistology. The invention further encompasses kits (e.g., diagnostic kits) comprising the antibodies described herein.

Unless otherwise defined herein, scientific and technical terms used in connection with the present invention shall have the meanings that are commonly understood by those of ordinary skill in the art. Exemplary methods and materials are described below, although methods and materials similar or equivalent to those described herein can also be used in the practice or testing of the present invention. In case of conflict, the present specification, including definitions, will control.

Generally, nomenclature used in connection with, and techniques of, cell and tissue culture, molecular biology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein.

Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers.

In order that this invention may be better understood, the following examples are set forth. These examples are for purposes of illustration only and are not to be construed as limiting the scope of the invention in any manner.

### EXAMPLES

### Example 1: Novel Anti-Gal3 scFvs Production

New galectin-3 inhibitors monoclonal antibodies were obtained using Mabqi human phage display platform.

Several constructs have been produced for the panning experiments: hu-Gal3-FL (full length human Galectin-3, as shown in SEQ ID NO 106, and available under UniProt Accession No. P17931—LEG3_HUMAN), hu-Gal3-CRD (CRD domain of human Galectin-3, comprised in SEQ ID NO 107) and m-Gal3-FL (full length mouse Galectin-3, as shown in SEQ ID NO 110, and available under UniProt Accession No. P16110—LEG3_MOUSE), which have been previously controlled and which showed no protein degradation.

Recombinant proteins were immobilized on a nunc maxisorp plate using via their histidine tag: streptavidin was coated at 10 μg/mL in TBS, 100 μL/well and incubated 1 hour at room temperature. Plate was then blocked for 2 hours with 200 μL of TBS-BSA 4%. A solution with 5 mM NiSO4 and Tris-NTA-Biotin 1 μg/mL in TBS was incubated for 30 minutes at room temperature. Plate was washed 3 times using TBST 0.1% and 100 μL of NiSO4/Tris-NTA-biotin solution was added to the wells for 1 hour at room temperature. Plate was washed again and recombinant proteins were added at 20 μg/mL in TBS, 100 μL/well and incubated overnight at 4° C. Plate was washed 3 times using TBST 0.1% and phage library was depleted on wells coated with streptavidin and NiSO4/Tris-NTA-biotin only: 50 μL of library was incubated with 50 μL of TBS-BSA 4% for 1 hour at room temperature.

Depleted library was incubated on antigen coated wells for 2 hours at room temperature. Wells were washed 10 times with TBST 0.1% and 3 times with TBS to remove non-specific phages.

Bound phages were eluted by adding 100 μL of trypsin-EDTA (Gibco, 25300-054) and incubated 15 minutes at room temperature. 200 μL of TG1 (OD=0.3-0.5) were infected with 100 μL of eluted phages for 1 hour at 37° C.

Infected TG1 were centrifuged 10 minutes at 4000 rpm, pellet resuspended in 2×TY, ampicillin, Glc 2% and culture incubated overnight at 30° C., 200 rpm. 20 μL of infected TG1 were added to 2 mL 2×TY, ampicillin, Glc 2% and grown at 37° C., 200 rpm. When OD reached 0.5, TG1 were infected with helper phage for 1 hour at 37° C., 200 rpm.

TG1 culture was centrifugated 10 minutes at 4000 rpm, pellet resuspended in 1 mL 2×TY, ampicillin, kanamycin and incubated overnight at 30° C., 200 rpm.

Culture was centrifuged at 4000 rpm for 15 minutes, 4° C. and phages precipitated for supernatant using PEG-NaCl. These phages were used for the next round of panning.

Several rounds of panning allowed the successful enrichment of multiple antigen binders.

Monoclonal scFvs were then sequenced and selected when unique clones.

Monoclonal scFvs have then been produced in E. coli. HB2151 bacteria containing scFv expressing pHEN phagemid were grown overnight at 37° C. 220 rpm in 150 μL 2TY—2% glucose—ampicillin in 96 well plates. This starter was used to inoculate larger cultures in deep well plates containing 2TY—IPTG 1 mM—ampicillin for scFv expression induction. Induction cultures were grown overnight at 30° C. 220 rpm. Cultures were centrifuged and polymyxin B sulfate 1 mg/mL in Tris 20 mM pH 8 was used for periplasmic extraction of scFv from pellets.

An overview of the SEQ ID numbers of the CDRs, VH and VL domains is provided below in Tables 1 and 2, as well as DNA sequences corresponding to said VH and VL sequences in Table 2.

TABLE 1
SEQ ID NOs for the H-CDR1-3 and L-CDR1-3 amino
acid sequences of the anti-Gal3 antibodies

| Antibody | H-CDR1 | H-CDR2 | H-CDR3 | L-CDR1 | L-CDR2 | L-CDR3 |
|---|---|---|---|---|---|---|
| D06 | 1 | 6 | 12 | 20 | 27 | 30 |
| G03 | 2 | 6 | 16 | 24 | 27 | 34 |
| H07 | 1 | 9 | 14 | 24 | 27 | 34 |
| H10 | 5 | 7 | 3 | 21 | 28 | 32 |
| B12 | 4 | 11 | 18 | 21 | 29 | 36 |
| D11 | 1 | 6 | 13 | 21 | 27 | 31 |
| E02 | 2 | 10 | 17 | 25 | 27 | 35 |
| E07 | 1 | 6 | 14 | 22 | 27 | 31 |
| E12 | 2 | 10 | 19 | 26 | 27 | 37 |
| E01 | 3 | 8 | 15 | 23 | 27 | 33 |

TABLE 2
SEQ ID NOs for the VH and VL protein and DNA sequences of the
anti-Gal3 antibodies

| Antibody | VH (Protein) | VL (Protein) | VH (DNA) | VL (DNA) |
|---|---|---|---|---|
| D06 | 38 | 39 | 61 | 62 |
| G03 | 48 | 49 | 71 | 72 |
| H07 | 50 | 51 | 73 | 74 |
| H10 | 44 | 45 | 67 | 68 |
| B12 | 54 | 55 | 77 | 78 |
| D11 | 40 | 41 | 63 | 64 |
| E02 | 52 | 53 | 75 | 76 |
| E07 | 42 | 43 | 65 | 66 |
| E12 | 56 | 57 | 79 | 80 |
| E01 | 46 | 47 | 69 | 70 |

### Example 2: Human/Murine Cross-Reactive Anti-Gal3 scFvs, Binding Neither Gall Nor Gal7

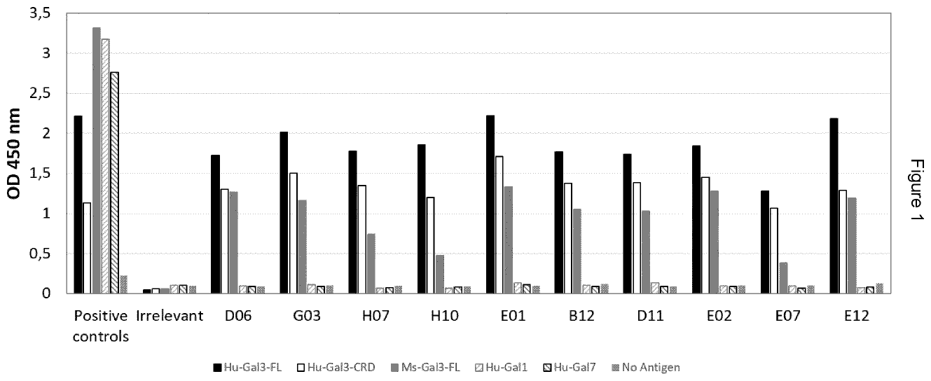

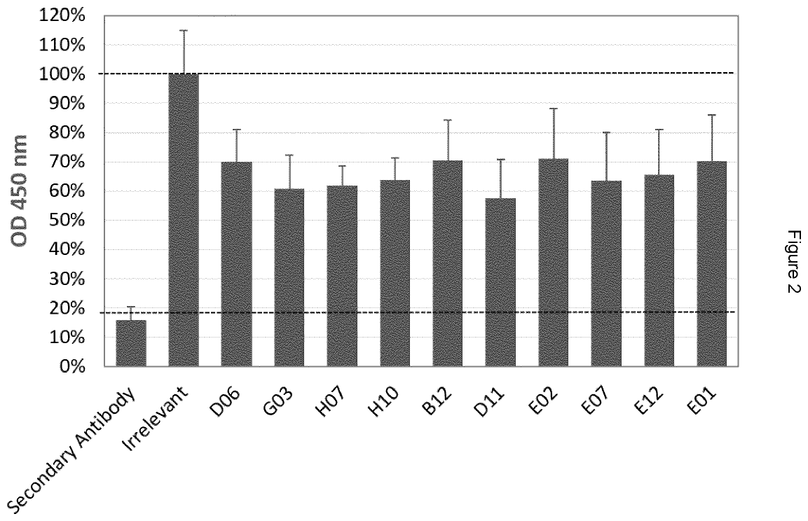

Binding cross-reactivity and specificity of scFvs were tested by ELISA on human Gal3 full-length and CRD domain (respectively Hu-Gal3-FL and Hu-Gal3-CRD), on human full-length Gall (Hu-Gall, as shown in SEQ ID NO 108, and available under UniProt Accession No. P09382—LEG1_HUMAN) and human full-length Gal7 (Hu-Gal7, as shown in SEQ ID NO 109, and available under UniProt Accession No. P47929—LEG7_HUMAN)), and on murine full-length Gal3 (m-Gal3-FL) (FIG. 1).

Hu-Gal3-FL, Hu-Gal3-CRD, m-Gal3-FL, Hu-Gall and Hu-Gal7 were immobilized at 3 μg/mL final concentration through their histidine tag on functionalized microplates following internal process. Periplasmic extracts containing scFvs were added into the wells and detected with an anti-scFvTag.HRP (horseradish peroxidase) antibody. Rabbit antibodies directed against Gal3 (ab53082-abcam reference), Gall (ab25138-abcam reference), and Gal7 (ab108623-abcam reference) were used at 5 μg/mL as antigen-coating positive controls and detected with an anti-rabbit.HRP secondary antibody (7074S-Cell Signaling reference).

All scFvs of the present invention show binding to both human and murine Gal3 proteins, and no binding to human Gall or Gal7.

### Example 3: Competitive Inhibition Assay of Gal3 Binding to Asialofetuin

Asialofetuin (ASF) glycoprotein is often studied as the model ligand of galectins. In this assay, the binding of ASF to immobilized human Gal3 (Hu-Gal3) is measured in the presence of scFvs or in their absence (controls with secondary antibody alone and with irrelevant scFvs). Human galectin-3 (Hu-Gal3-FL) was immobilized at 3 μg/mL through its histidine tag on functionalized microplates as before. ASF (A1908-Sigma reference) was added at 30 μg/mL in each well with or without periplasmic extracts containing the scFvs. ASF binding to Hu-Gal3 was detected with a sheep anti-ASF antibody (secondary antibody, ab35184-abcam reference) and a donkey anti-sheep.HRP antibody (tertiary antibody, ab6900-abcam reference).

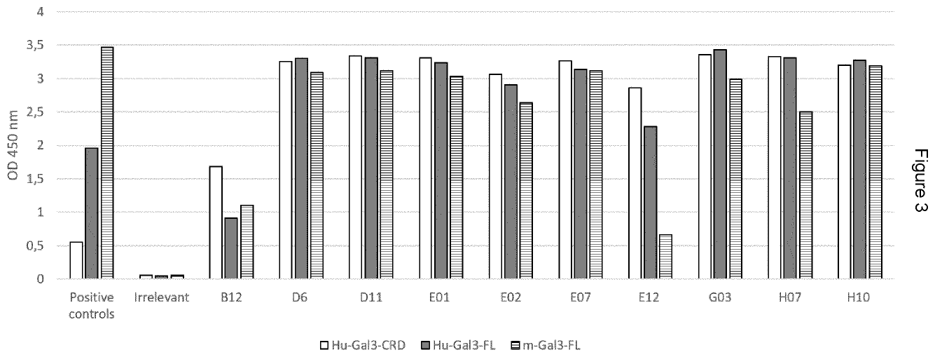

All of the scFvs of the present invention showed an inhibition of ASF-Gal3 interaction except for the irrelevant scFvs (FIG. 2).

### Example 4: Novel Anti-Gal3 Antibodies Production and Design of Anti-Gal3 Antibodies with Reduced Immunogenicity

Anti-Gal3 antibodies were then produced in IgG1 format using transient transfection in CHO-K1 cell line. Supernatants were harvested by centrifugation, filtered and subsequently purified using MabSelect SuRe™ (Cytiva).

Then, anti-Gal3 antibodies with reduced immunogenicity were designed in order to obtain antibody molecules having minimal immunogenicity when administered to humans, while substantially retaining the specificity and affinity of the parental antibodies.

VH and VL regions of the antibodies were blasted against human IgG databases in order to find the closest human germline sequence. Besides, sequences were run through EpiAnalyzer tool which highlighted immunogenic regions. It was observed that immunogenicity does not exceed tolerable levels.

After alignments of VH or VL sequences and germline sequences, variant antibodies “-G” were proposed correcting framework mutations to germline, whereas two variant antibodies, -G1 and -G2, were proposed in borderline cases where mutations are close to the CDR regions, G1 version having framework mutation to germline not being in proximity to the CDR regions and G2 including those in proximity to the CDR regions. SEQ ID numbers of variant antibodies VH and VL are provided in Table 3.

TABLE 3
SEQ ID NOs for the VH and VL protein sequences of the anti-Gal3
variant antibodies

| Antibody | VH | VL |
|---|---|---|
| D06-G1 | 81 | 83 |
| D06-G2 | 82 | 83 |
| D11-G1 | 84 | 86 |
| D11-G2 | 85 | 86 |
| E07-G1 | 87 | 89 |
| E07-G2 | 88 | 89 |
| H10-G | 90 | 91 |
| E01-G | 92 | 93 |
| G03-G1 | 94 | 96 |
| G03-G2 | 95 | 96 |
| H07-G | 97 | 98 |
| E02-G | 99 | 100 |
| B12-G | 101 | 102 |
| E12-G1 | 103 | 105 |
| E12-G2 | 104 | 105 |

### Example 5: Human/Murine Cross-Reactive Anti-Gal3 Antibodies

Anti-Gal3 antibodies human/mouse cross-reactivity was tested by ELISA on human Gal3 full-length (Hu-Gal3-FL) and CRD domain (Hu-Gal3-CRD), and on murine Gal3 (m-Gal3-FL).

Hu-Gal3-FL, Hu-Gal3-CRD and m-Gal3-FL were immobilized at 3 μg/mL via their histidine tag on functionalized microplates. Purified IgGs were added into the wells (15-20 μg/mL final concentration) and detected with an anti-Fab.HRP antibody. A control rabbit antibody directed against Hu-Gal3 (Abcam ab53082 reference) was used at 5 μg/mL as antigen-coating positive control and detected with an anti-rabbit.HRP secondary antibody (7074S-Cell Signaling reference).

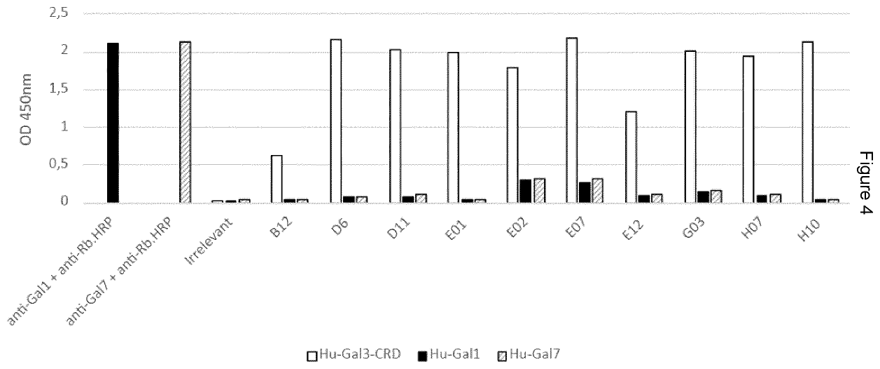

The results are presented on FIG. 3. Antibodies of the present invention show binding to both human and murine Gal3 proteins. Lower binding signals on human and mouse Gal3 were however observed for B12 antibody. Also, the signal of E12 antibody was much lower on the m-Gal3 than on the Hu-Gal3-FL and Hu-Gal3-CRD.

### Example 6: Antibodies Binding Specifically to Gal3, and Neither to Gall Nor to Gal7

IgG specificity to Gal3 was tested by checking the absence of binding on human Gall and Gal7 by ELISA.

Hu-Gall and Hu-Gal7 were immobilized at 3 μg/mL. Purified IgGs were added into the wells (15-20 μg/mL final concentration) and detected with an anti-Fab.HRP antibody. Rabbit antibodies directed against Gall (Abcam ab25138 reference) and Gal7 (Abcam ab108623 reference) were used at 5 μg/mL as antigen-coating positive controls and detected with an anti-rabbit.HRP secondary antibody (7074S-Cell Signaling reference).

ELISA results are presented on FIG. 4. All anti-Gal3 antibodies of the present invention specifically bind to Gal3 and do not bind to human Gall and Gal7.

### Example 7: Affinities of the Antibodies to Gal3 from Several Species

The interaction of the anti-Gal3 antibody with recombinant human Gal3 full-length (Hu-Gal3-FL) and murine Gal3 (m-Gal3-FL) (from Novalix) was monitored by surface plasmon resonance (SPR) detection using a Biacore T-200 instrument.

Binding studies were performed in freshly prepared, filtered and degassed running buffer containing 10 mM HEPES, 150 mM NaCl, and 0.05% P20 (HBS-P, Cytiva Life Sciences) at 25° C. Before performing the binding studies, the CM5 sensor surface immobilized with anti-human Fc mAb was equilibrated with HBS-P by priming the instrument at least three to four times.

The CM5 sensor surface was first immobilized with an anti-human Fc mAb using a standard protocol. The entire sensor surface was first activated by injecting a 1:1 (v/v) mixture of 400 mM EDC and 100 mM NHS at a flow rate of 10 μL/minute for 7 minutes. Monoclonal anti-human Fc antibody (Millipore AP113), 25 μg/mL in Na acetate pH5, 360 s were flowed at 10 μL/minute to reach 10000 RU on both flow cells followed by the injection of 1 M ethanolamine, pH8.5 for 7 minutes at a flow rate of 10 μL/minute. During the entire immobilization procedure, HBS-P was used as the running buffer.

Antibody samples were prepared at 5 μg/ml and were injected for 30 seconds at 10 μL/minute. In general, 400 to 680 resonance units of antibodies were captured on the chip.

The detailed Kd values for each tested antibody is represented below in Table 4.

TABLE 4
Determination of Kd values on human and mouse full-length Gal3

| | Kd on human Gal3 | Kd on murine Gal3 |
|---|---|---|
| Antibody | (M) | (M) |
| D06 | 1.3E−08 | 1.34E−09 |
| D06-G1 | 9.6E−09 | Not done |
| D06-G2 | 6.8E−09 | Not done |
| G03 | 6.00E−09 | 4.53E−09 |
| H07 | 5.65E−09 | 4.86E−09 |
| H10 | 4.5E−09 | 2.72E−09 |
| H10-G1 | 4.9E−09 | Not done |
| B12 | 8.80E−09 | 3.47E−09 |
| D11 | 1.5E−08 | 2.80E−09 |
| D11-G1 | 1.5E−08 | Not done |
| D11-G2 | 1.8E−08 | Not done |
| E02 | 7.38E−09 | 4.13E−09 |
| E07 | 9.0E−09 | 4.14E−09 |
| E07-G1 | 1.2E−08 | Not done |
| E07-G2 | 1.5E−08 | Not done |
| E12 | 6.64E−09 | 7.70E−09 |
| E01 | 6.26E−09 | 7.81E−09 |

On human Gal3, native antibodies of the present invention show Kd values between 4 nM and 15 nM and variants antibodies show values between 4 nM and 18 nM. On mouse Gal3, only native antibodies have been tested and they show lower Kd values between 1 nM and 8 nM. There are no significant difference in Kd values between human and mouse antigen.

Kd values are also similar for variants antibodies compared to native antibodies. These similar binding characteristics show that mutations in the framework of VH or VL domains do not have an impact on the interaction of the antibody to human Gal3.

Kd values were also determined by SPR using single-cell kinetics analysis. A total of 5 concentrations of Gal3 CRD domain from human, dog, rat and cynomolgus (Novalix) [0.5-50] nM were injected over the antibody capture surfaces for 210 seconds followed by a dissociation phase of 600 seconds at 30 μL/minute. At the end of each cycle, capture surfaces were regenerated by 2 injections of glycine pH 1.5, for 30 seconds at 10 μL/minute, followed by an extra wash with glycine pH 1.5. Fresh antibody was captured at the beginning of each cycle.

Kd values for each tested antibody is indicated in Table 5 below.

TABLE 5
Determination of Kd values on human, rat, cynomolgus and dog
Gal3-CRD

| | | | Kd on | |
|---|---|---|---|---|
| | Kd on human | Kd on rat | cynomolgus | Kd on dog |
| Anti- | Gal3-CRD | Gal3-CRD | Gal3-CRD | Gal3-CRD |
| body | (M) | (M) | (M) | (M) |
| D11 | 2.6E−08 | 8.6E−09 | 1.1E−08 | 2.6E−08 |
| E07 | 1.4E−08 | 1.5E−08 | 7.4E−09 | 2.3E−08 |
| D06 | 1.5E−08 | 4.8E−09 | 1E−08 | 1.2E−08 |
| H10 | 8.2E−09 | 1.2E−08 | 5.6E−09 | 2.4E−08 |

### Example 8: EC50 Determination by ELISA on Human and Mouse Gal3

To determine the half maximal effective concentration (EC50) of the antibodies, ELISA dose-response experiments were performed on Hu-Gal3-FL and on m-Gal3-FL.

Hu-Gal3-FL and m-Gal3-FL were immobilized at 3 μg/mL through their His-tag on functionalized microplates as before. A 3.3-fold serial dilution was carried-out with the purified IgGs, added to the wells containing immobilized Hu-Gal3-FL or m-Gal3-FL and detected with an anti-Fab.HRP antibody.

Human/mouse Gal3 EC50 ratio were estimated according to EC50 values (sigmoidal dose-response equation: Y=Bottom+X*(Top−Bottom)/(EC50+X)), a low ratio indicating a high cross-reactivity.

EC50 were measurable for eight out of the ten antibodies tested. EC50 for B12 and E12 antibodies were too high to be determined. Among the eight remaining antibodies, six had EC50 values below 10 nM on human Gal3 and four on mouse Gal3. Human/mouse Gal3 EC50 ratio were below four for D06, D11, E02, E07 and E01 antibodies, confirming a high cross-reactivity.

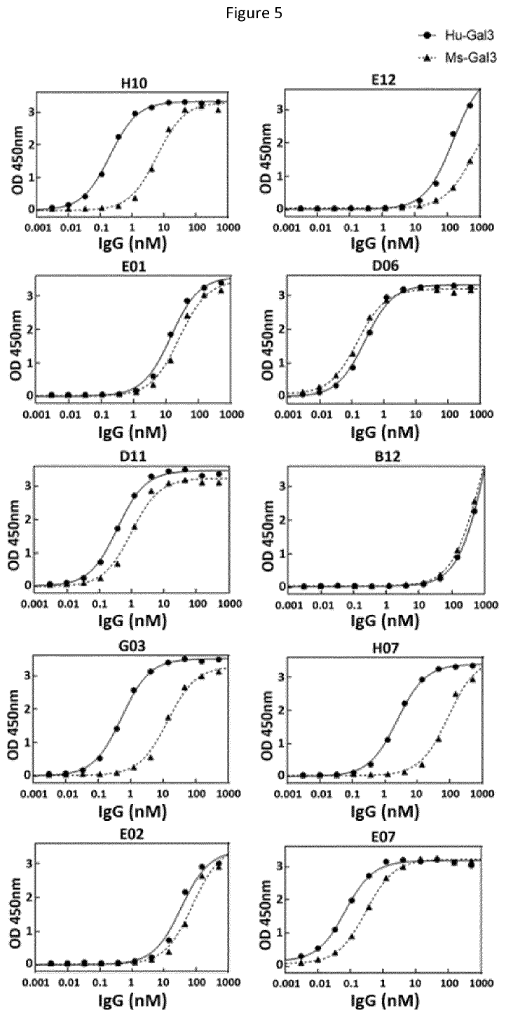

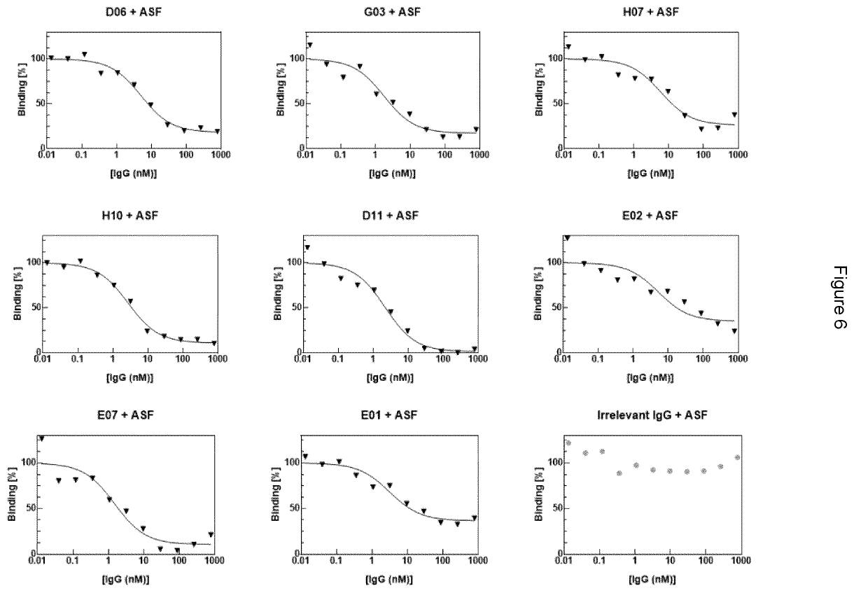

Results are presented in FIG. 5 and Table 6 below.

TABLE 6
EC50 calculation on human and mouse Gal3

| Antibody | hGal3 EC50 (nM) | mGal3 EC50 (nM) | Ratio |
|---|---|---|---|
| D06 | 0.26 | 0.16 | 2 |
| G03 | 0.52 | 13.6 | 26 |
| H07 | 2.4 | 88 | 37 |
| H10 | 0.20 | 5.9 | 30 |
| D11 | 0.37 | 0.99 | 3 |
| E02 | 34 | 76 | 2 |
| E07 | 0.07 | 0.30 | 4 |
| E01 | 14.3 | 25.9 | 2 |

### Example 9: Competitive Inhibition Assay of Gal3 Binding to Asialofetuin and IC50 Determination on Human and Mouse Gal3

The inhibitory properties and IC50 of the anti-Gal3 antibodies were determined in an ELISA competitive assay by measuring the binding of ASF to immobilized CRD domain of human Gal3 in absence or presence of the antibodies at different concentrations.

The inhibition assay was performed on Hu-Gal3-CRD which was immobilized at 1 μg/mL through its His-tag on functionalized microplates. A 2-fold or 3-fold serial dilution was carried-out with the purified IgGs and added to the wells with immobilized Hu-Gal3-CRD. ASF (A1908-Sigma reference) was added at 8 μg/mL in each well with or without IgGs. ASF binding to Hu-Gal3-CRD was detected with the HRP-labeled anti-ASF sheep antibody. IC50 were estimated according to the model equation Y=Bottom+(Top−Bottom)/(1+(X/IC50)). Indicative maximal percentages of inhibition were obtained with the formula: 100%−(((mean of bottom plateau values of IgGs)×100%)/mean of irrelevant IgG values)

All, except the negative control (the irrelevant IgG), showed inhibition of ASF binding to Hu-Gal3-CRD (FIG. 6). IC50 of the antibodies ranged from 1.5 to 6 nM and estimated relative maximal inhibition percentages ranged from 55 to 97%, as shown in Table 7 below.

Therefore, these 8 antibodies are antagonists of Gal3/ASF binding.

TABLE 7
IC50 calculation on human Gal3 CRD

| Antibody | hGal3-CRD IC50 (nM) | Max % inhibition |
|---|---|---|
| D06 | 5.1 | 82 |
| G03 | 1.8 | 83 |
| H07 | 6.0 | 74 |
| H10 | 2.7 | 89 |
| D11 | 2.2 | 99 |
| E02 | 5.2 | 65 |
| E07 | 1.5 | 89 |
| E01 | 3.2 | 63 |

### Example 10: Inhibitory Activity of the Antibodies in a Hypochlorous Acid HOCI-Induced Murine Model of Fibrosis

The therapeutic effect of anti-Gal3 antibodies on lung and skin fibrosis was evaluated in a murine model of HOCI-induced fibrosis in two independent experiments.

In a first experiment, control mice (n=12) received a daily intradermal injection of 300 μL of sterile phosphate buffer saline (PBS) for 5 consecutive days per week, for a duration of 42 days. HOCI alone and HOCI with antibody (HOCI-antibody) treated mice (n=12 per group) received a daily intradermal injection of 300 μL of HOCI for 5 consecutive days per week, for a duration of 42 days. Antibody-treated mice also received a subcutaneous injection of 20 mg/kg antibody, starting at day minus one and every 5 days between day 5 and day 40. Each antibody was solubilized in sterile, endotoxin-free PBS before injection. In a second experiment, the same protocol was applied, except that duration of the study was 63 days and antibodies were given every 7 days starting at day minus 1 until day 62.

Mice that received daily injection of HOCI were also treated by intratracheal instillation of 0.5 mg/kg of TD139 compound (HOCI-TD139), a small molecule Gal3 inhibitor. In a first experiment, the administrations started from day 21 and the compound was given every 3 days until day 39. In a second experiment, the administrations started from day 20 and the compound was given every 3 days until day 62. TD139 was initially dissolved in 100% DMSO then diluted 50-fold in endotoxin-free PBS before administration.

### A—Longitudinal Evaluation of Skin Thickness

The evolution of skin thickness was monitored by using an external caliper, every seven days until sacrifice.

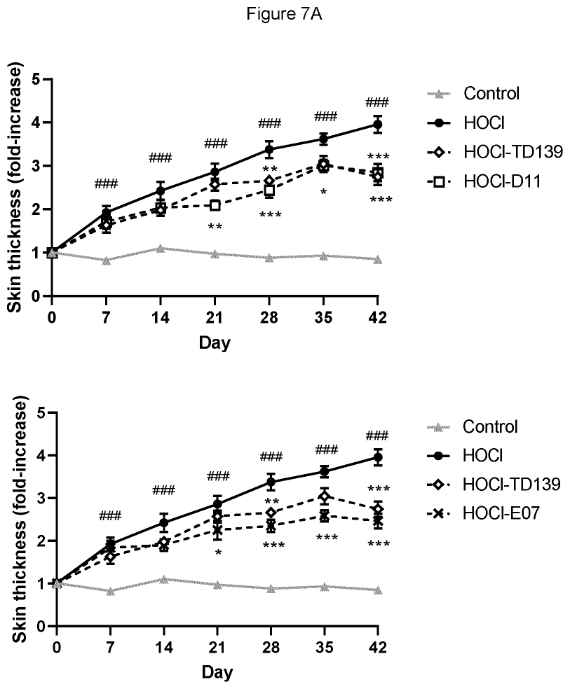

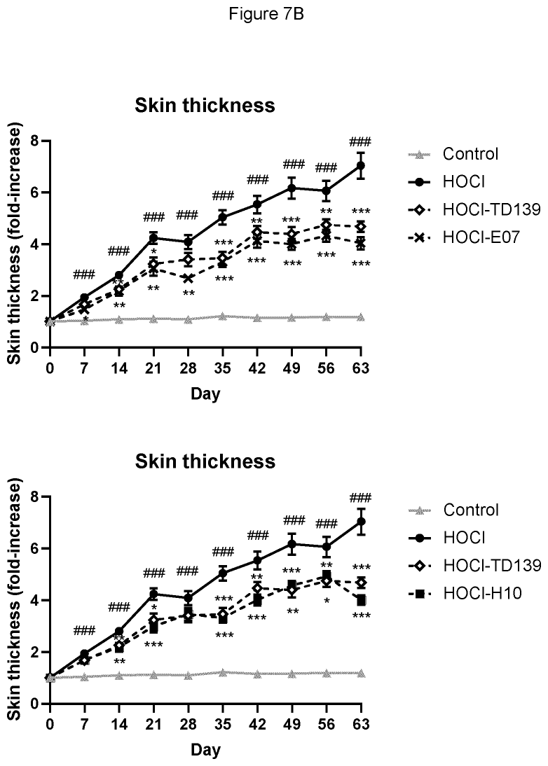

In the first experiment as shown in FIG. 7A, the daily HOCI injection was associated with a rapid, sustained and statistically significant increase in skin thickness compared to control mice, starting from day 7, and reaching a maximum of 3.9 fold at day 42. The HOCI-induced increase in skin thickness was significantly reduced by both D11 and E07 monoclonal antibodies starting from day 21, with a sustained effect until day 42. Mice receiving the reference item TD139 showed a lower decrease in skin thickness than with either antibody, reaching statistical significance only at days 28 and 42.

In the second experiment, the sustained and significant increase in skin thickness in HOCL-treated mice, compared to control mice, started from day 7 and reached a maximum of 5.9 fold at day 63 (FIG. 7B). The HOCI-induced increase in skin thickness was significantly reduced by E07 starting from day 7 and by H10 starting from day 14, with a sustained effect until day 63.

Consequently, the antibodies of the present invention have significant ability to decrease the fibrosis indicator of skin thickness measured longitudinally, at a similar or higher extent than TD139 (FIGS. 7A and 7B), for at least 9 weeks using a weekly scheme of administration (FIG. 7B).

### B—Quantification of Skin and Lung Collagen

At the day of study termination of the first experiment (day 42), skin and lung samples were taken, formalin-fixed and processed to individual paraffin wax blocks. Sections of skin and lung were cut from the blocks and, after rehydration, stained with Picrosirius Red coloration according to the manufacturer's protocol. Subsequently, each slide was examined and remotely analyzed employing the web-based Image J software

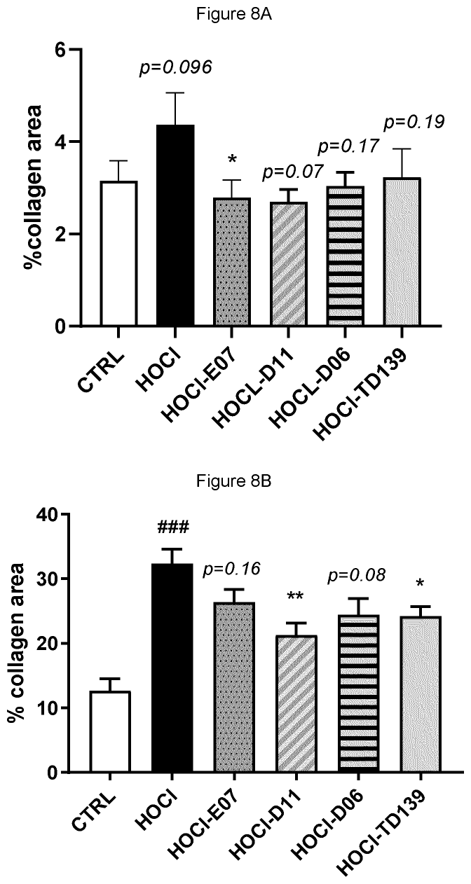

The daily intradermal injection of HOCI was associated with an increase of collagen deposition compared to vehicle mice in lungs (FIG. 8A) and in skin (FIG. 8B).

The subcutaneous injection of D06, D11 and E07 antibodies, as well as the intratracheal instillation of the reference compound TD139 restored lung collagen levels to those of control mice, reaching statistical significance for E07.

The subcutaneous injection of D11 antibody and reference compound TD139 significantly reduced the levels of skin collagen compared to the HOCI group. A non significant downward trend was also observed after treatment with E07 and D06 antibodies.

Consequently, the antibodies of the present invention have clear ability to decrease the fibrosis indicators of collagen deposition, both in lungs and skin, as measured histologically after 6 weeks of treatment.

### C—Effect on Plasma Interleukin-6 (IL-6) and Interleukin-5 (IL-5) Levels in Mice

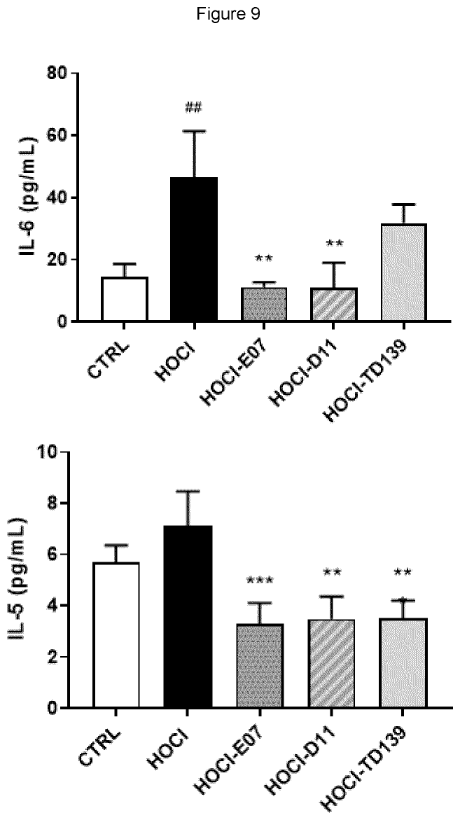

In the first experiment, a mixed effect model with repeated measurements was performed on Control group, HOCI treated group, HOCI-antibody- and HOCI-TD139-treated groups. The impact of HOCI administration on plasma IL-6 and IL-5 levels was evaluated compared to the control group. The impact of antibody and TD139 treatments was compared to the HOCI group (FIG. 9). IL-6 and IL-5 were quantified by using the MSD technology from Mesoscale.

The plasma levels of IL-6 were significantly increased after HOCI administration. Treatment with E07 or D11 antibodies significantly decreased the plasma IL-6 levels to values similar to those of control mice, whereas TD139 had no activity. Consequently, the antibodies of the present invention have superior ability than TD139 to decrease the levels of IL-6 in plasma after HOCI treatment.

E07 and D11 antibodies, as well as TD139 also decreased significantly the plasma IL-5 levels compared to the HOCI group.

### D—Antibody Treatment Resets Gal3 Plasma Levels to Those of Healthy Mice

In the first experiment, the plasma levels of Gal3 were measured at day minus 1 (before antibody administration) and at termination day (day 42) using a mouse Galectin-3 DuoSet ELISA kit (R&D Systems, DY1197) according to the manufacturer's instructions. Plasma samples were diluted 1:200 in order to fit with the dynamic range of the assay.

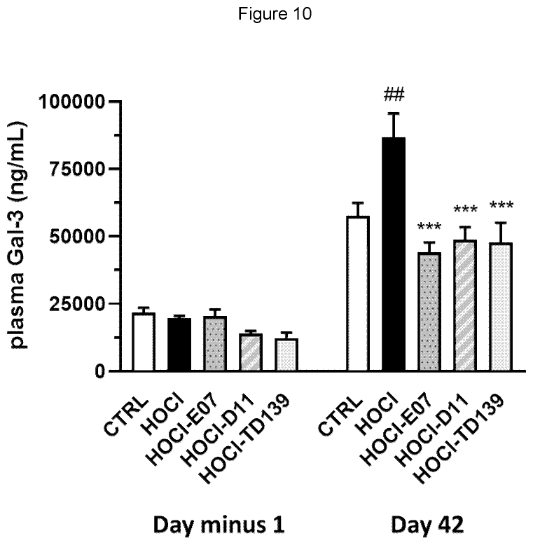

At day minus 1, as expected, no significant difference was observed between all groups (FIG. 10). At day 42, a significant increase of Gal3 was observed in plasma of HOCI-treated mice. Treatment with E07 and D11 antibodies, as well as with TD139 compound, restored the plasma Gal3 levels to values similar to those of the control group.

E—Genes Dysregulated in Pathological Conditions are Virtually Fully Normalized after Treatment with Anti-Gal3 Antibody

In the first experiment, global gene expression was examined at day 42 in whole blood cells by RNA sequencing.

Whole blood was collected in RNA protect tubes (Qiagen) and were processed for RNA extraction using the RNeasy Protect Animal blood kit with the optional on-column DNAse I digestion. Total RNA samples were quantified on Nanodrop 2000 (ThermoFisher Scientific™) and analyzed on Bioanalyzer (Agilent) using RNA 6000 Nano chips to determine RNA quality (RIN). Total RNA matching the QC criteria were included in the study. Purified RNA samples were used as input for the TruSeq Strand-specific RNA-seq (poly A selection and globin depletion) according to manufacturer's protocol (Illumina). Following libraries preparation, samples matching the requested criteria were sequenced on a NovaSeq 6000 sequencer (Illumina).

Raw reads were processed using the in-house RNAExp Expression pipeline 2.1 and the quality was assessed using FastQC tools. Then, reads were trimmed for adaptors using Cutadapt and trimmed reads were aligned to mouse mm39 reference genome using the STAR aligner. Aligned data were evaluated for quality using Samtools and Picard tools. Then, aligned reads per gene were quantified using FeatureCount. The sample filtering step identified 2 outliers, one in control and one in TD139-treated group which were discarded for further analysis. Read count were normalized by the variance stabilizing transformation vst function from DESeq2 R package.

55,416 genes were detected in the data. 29,947 non protein-coding genes were removed. Those with 0 count over all the samples or having an expression level below 1 in more than 95% were filtered. At the end, RNA-Seq data comprised 10,508 genes. Genes differentially expressed between groups were determined using a linear model (ImFit function from limma R package) on vst transformation gene expression dataset. Resulting p-values were adjusted for multiple hypothesis testing and filtered to retain differentially expressed genes with a False Discovery Rate (FDR) adjusted p-value<0.05 and a Fold-Change (FC) value≥1.3.

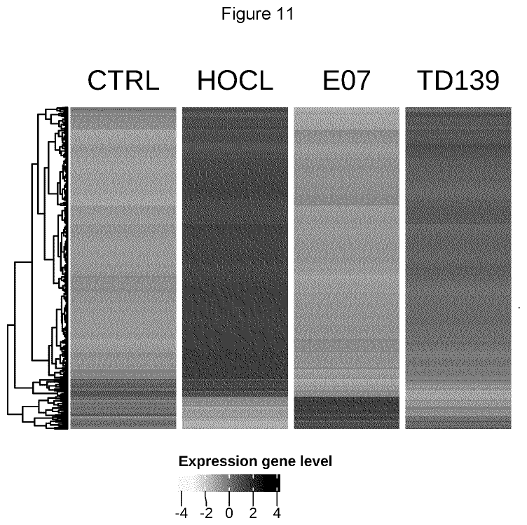

510 genes were found differentially regulated between the pathological HOCI group and the control group (484 up, 26 down). Treatment with E07 antibody counteracted this pathological pattern for 445 representing 87.2% of these genes, leading to a global gene expression profile very close to that of the control group (FIG. 11). By contrast, only 7 genes representing 1.37% of those dysregulated between the HOCI and control group were found counteracted after treatment with the reference compound TD139. Consequently, E07 antibody of the present invention shows clear superiority over TD139.

### Example 11: Activity of the Antibodies in a Bleomycin-Induced Murine Model of Lung Fibrosis

The therapeutic effect of anti-Gal3 antibodies on lung gene expression and lung fibrosis was evaluated in a murine model of bleomycin-induced fibrosis.

Control bleomycin mice named ‘Bleo’ received an administration of 1.65 mg/kg bleomycin by the oropharyngeal route on Day 1. Control non challenged mice, named ‘CTRL’ or ‘control’, did not receive bleomycin but the saline vehicle of bleomycin on day 1. Antibody-treated mice also received an administration of 1.65 mg/kg bleomycin by the oropharyngeal route on Day 1 and a subcutaneous injection of 20 mg/kg antibody, starting on day minus one and on days 4, 8, 12, 16 and 20. Each antibody was solubilized in sterile, endotoxin-free PBS before injection. Mice that received bleomycin were also treated by oropharyngeal administration of 0.5 mg/kg of TD139 compound (Bleo-TD139), a small molecule Gal3 inhibitor, starting on day 12 and every 2 days until day 20. TD139 was initially dissolved in 100% DMSO.

### A—Evolution of the Body Weight

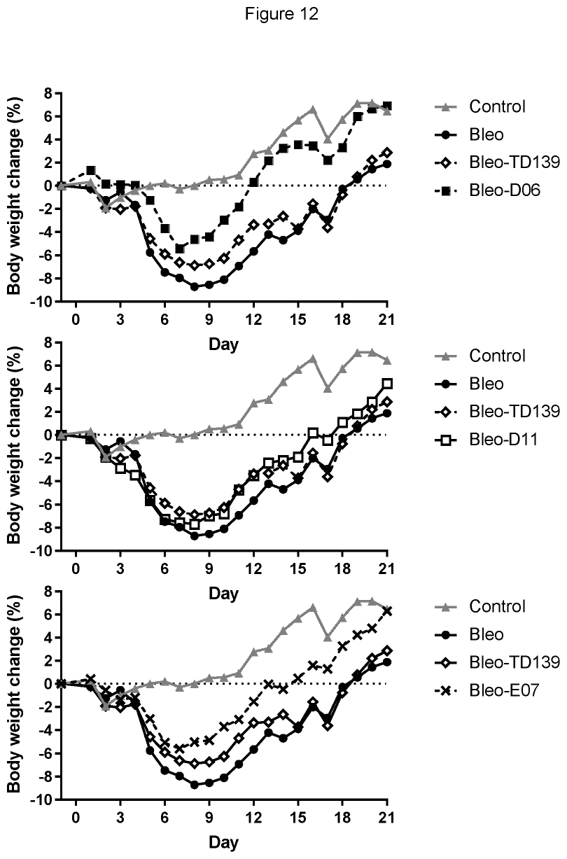

Mice were weighed daily. Their growth curves are represented on FIG. 12. Control mice had normal growth with a mean percent increase of 6.4% of their initial body weight measured on day minus 1. Mice treated with bleomycin showed a rapid decrease of their body weight reaching a maximum of 8.7% of weight loss on day 8, followed by a phase of weight increase from day 9 until day 21. The subcutaneous injection of antibodies D06 and E07 allowed a more rapid recovery of body weight, reaching values close to those of control mice from day 12 and day 18, respectively, and full weight recovery on day 21. D11 antibody showed a mean percent weight increase of 4.4% on day 21 compared to day minus 1. TD139 compound showed more modest efficacy on this parameter, with a mean percent weight increase of 2.9% on day 21 compared to day minus 1, slightly higher than for the untreated bleomycin group (+1.9%).

These results demonstrate that antibodies of the present invention acted by improving the global animal welfare reflected by an improvement of their body weight.

### B—Body Weight Metrics

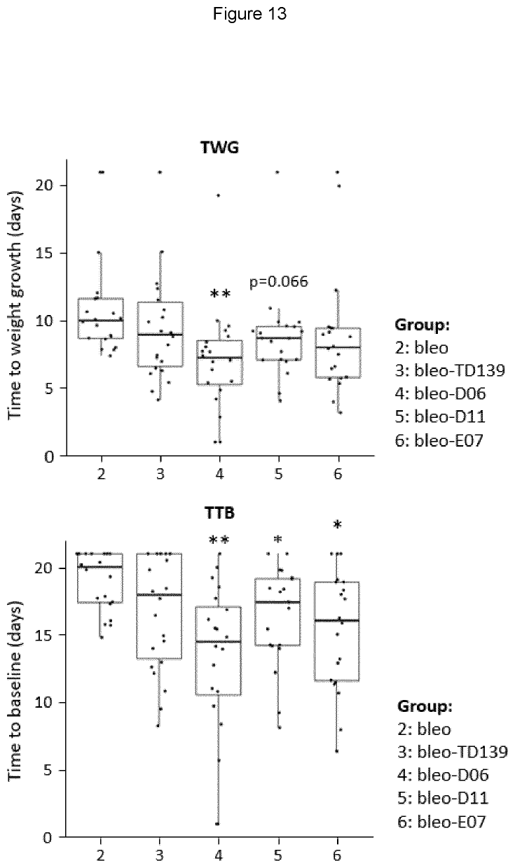

Body weight metrics were assessed by using two parameters called Time to weight growth (TWG) and Time to baseline (TTB), shown on FIG. 13. Time to weight growth corresponds to the point of inflection when mice show an ascending growth curve and start to regain weight within the course of the experiment. Time to baseline corresponds to the number of days required for mice to recover their initial body weight (day minus 1). The median TWG was 10 days for the untreated bleomycin group. It was significantly reduced by 2.8 days after treatment with D06 antibody and was also reduced by 1.3 and 2 days after treatment with D11 and E07 antibodies, respectively, with p-values just above the limit of statistical significance. TD139 compound had no efficacy on this parameter (p-value 0.53).

The median TTB was 20 days for the untreated bleomycin group. It was significantly reduced by 5.5 days, 2.7 days and 4 days after treatment with D06, D11 and E07 antibodies, respectively, and was also reduced by 2.7 and 4 days after treatment with D11 and E07 antibodies, respectively, with p-values just above the limit of statistical significance. TD139 compound had no efficacy on this parameter (p-value 0.29).

These results demonstrate that antibodies of the present invention acted by improving the global animal welfare reflected by an improvement of their body weight metrics

### C—Measurement of the Extent of Lung Fibrosis

Mice were submitted for terminal euthanasia on day 21. The lungs were collected for histopathological examination and gravity inflation-fixed with 10% neutral buffered formalin (NBF) before being immersion fixed in NBF.

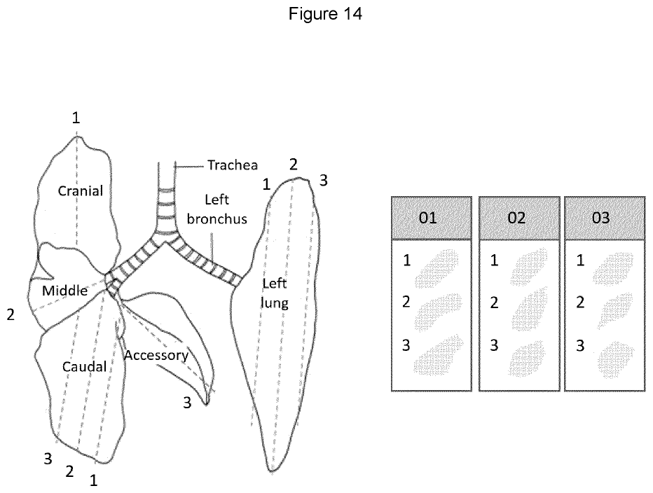

Sections were taken and then placed on three slides as indicated by the code numbers on FIG. 14.

Three longitudinal sections parallel to and bisecting the main stem bronchus were trimmed from the left lung and right caudal lung lobes and mounted in cassettes 01 and 02, respectively. The right cranial, middle and accessory lung lobes were sectioned longitudinally and mounted in cassette 03. All sections were stained with picrosirius red and scored in a blinded manner by a certified veterinary pathologist using the Modified Ashcroft fibrosis score (0-8), where “0” corresponds to “normality (no fibrosis) and “8” corresponds to the highest possible fibrosis score, as described by Hübner et al. (2008) but based on assessment across the whole section of lung.

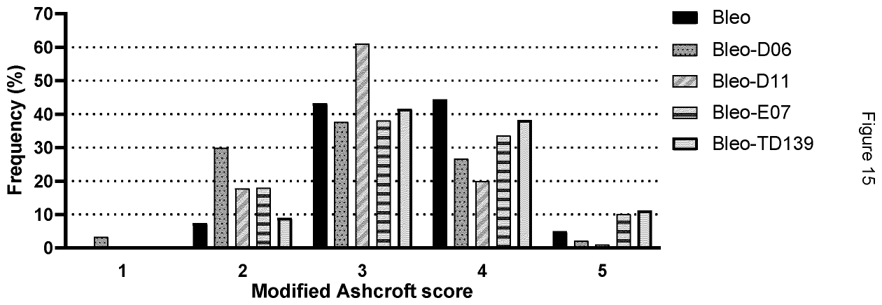

87.6% of the animals in mice having received bleomycin without any treatment had median fibrosis score of 3 or 4 and only 7.4% had a score of 2 (FIG. 15).

In mice treated with D06 antibody, the frequency of animals with media fibrosis scores of 3 or 4 was reduced to 64.5% while 30% of the animals had a low fibrosis score of 2.

Mice treated with D11 antibody also showed an improvement in the frequency of a score of 2 (17.8%), and there was a marked shift in the percent of mice that scored 4 or 5 towards lower scores compared to the untreated bleomycin group. In particular, the frequency of animals with a score of 4 was reduced to 20% compared to 44.4% in the bleo group and the frequency of animals with a score of 3 increased to 61.1% compared to 43.2% for the bleo group.

TD139 compound did not show any sign of anti-fibrotic efficacy, with 49.4% of mice having a lung fibrosis score of 4 and 5, compared to 49.3% for the untreated bleomycin group, and 50.6% of mice having a lung fibrosis score of 2 and 3, similar to that the untreated bleomycin group.

In conclusion, there was a clear evidence for attenuation of lung fibrosis in mice that received D06 antibody and to a lesser extent in mice that received D11 antibody.

### Example 12: Inhibitory Inflammatory Response of the Antibodies in Rat Model of ARDS

The therapeutic effect of anti-Gal3 antibodies on inflammation was evaluated in rat model of ARDS (Acute Respiratory Distress Syndrome).

Positive control rats received 3 doses of sterile phosphate buffer at day minus 11, minus 6 and minus 1 prior LPS challenge (0.25 mg/kg by subcutaneous route at day 1). Negative control rats received one oral dose of Dexamethasone at 10 mg/kg (dissolved in 0.5% carboxymethyl cellulose in deionized water) 2 hours prior LPS challenge (0.25 mg/kg by subcutaneous route at day 1). LPS (lipopolysaccharide) causes an acute inflammatory response, whereas dexamethasone has anti-inflammatory effects thus being the reference treatment in this model.

Antibody-treated rats received 3 doses of antibodies (14 mg/kg) at day minus 11, minus 6 and minus 1 prior to LPS challenge (0.25 mg/kg by subcutaneous route at day 1). Each antibody was solubilized in sterile PBS before injection.

Control rats of the experiment received 3 doses of sterile phosphate buffer at day minus 11, minus 6 and minus 1 without LPS challenge at day 1.

For all rats, plasma sampling was performed 4 h following LPS challenge.

### A—Effect on Neutrophils Counts Un BronchoAlveolar Lavage Fluid (BALF)

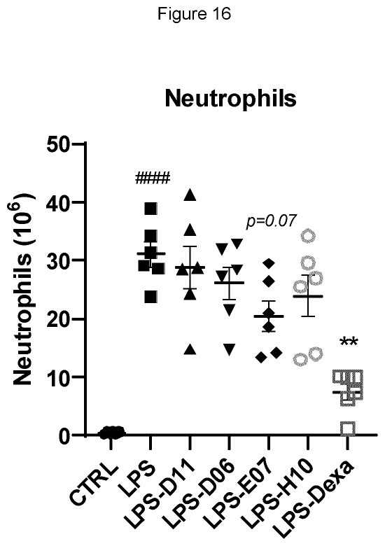

The impact of anti-Gal3 antibody treatment in LPS challenged rats were evaluated by quantification of neutrophils in Bronchoalveolar Lavage Fluid and compared to the positive control group (LPS challenge with PBS treatment) (FIG. 16). In rats of control group, the neutrophils count were significantly increased after LPS challenge, indicating that the rats produced an inflammatory reaction in response to LPS challenge. Neutrophils numeration levels were decreased after treatment with anti-inflammatory dexamethasone. In rats treated with anti-Gal3 antibodies D11, D06, E07 and H10, neutrophils counts were also decreased.

### Effect on Damage to the Air-Blood Barrier (BAL Protein Concentration)

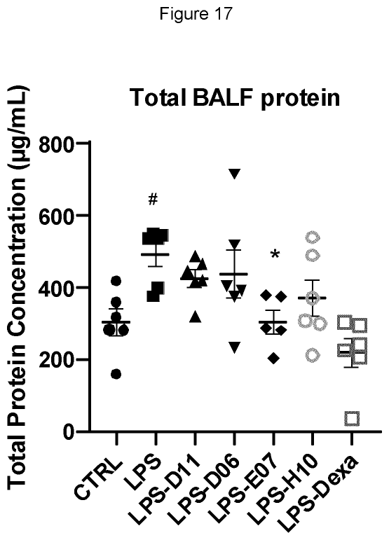

The impact of anti-Gal3 antibody treatment in LPS challenged rats were evaluated by quantification of proteins (by BCA assay) in BALF and compared to the positive control group (LPS challenge with PBS treatment) (FIG. 17). The presence of protein in BALF reflects the damage to the air-blood barrier. In rats of control group, the total BALF protein were significantly increased after LPS challenge, indicating that there is a rapid damage to the air-blood barrier in this group. Total BALF protein numeration levels were decreased after treatment with anti-inflammatory dexamethasone. In rats treated with anti-Gal3 antibodies D11, D06, E07 and H10, total BALF protein were also decreased with a statistic significance in the E07 group.

### B—Effect on Plasma TNF Alpha and KC-GRO Levels in Rats

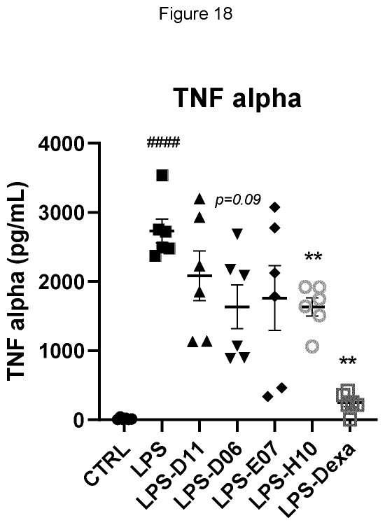

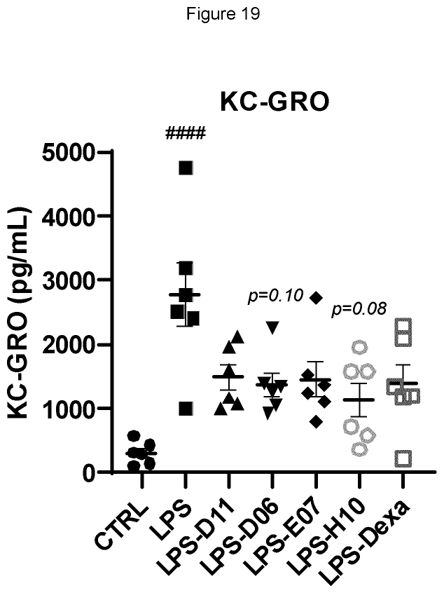

The impact of anti-Gal3 antibody treatment in LPS challenged rats were evaluated on plasma cytokine levels, namely TNF alpha and KC-GRO cytokines, and compared to the positive control group (LPS challenge with PBS treatment) (respectively FIG. 18A and FIG. 18B). TNF alpha and KC-GRO were quantified by using the MSD technology from Mesoscale.

In rats of control group, the plasma levels of TNF alpha were significantly increased after LPS challenge, indicating that the rats produced an inflammatory reaction in response to LPS challenge. TNF alpha levels were decreased after treatment with anti-inflammatory dexamethasone. In rats treated with anti-Gal3 antibodies D11, D06, E07 and H10, TNF alpha plasma levels were also decreased, displaying an intermediary profile between LPS challenged rat and LPS challenged rat treated with dexamethasone.

KC-GRO plasma levels were also reduced after treatment with the four antibodies at a similar extent than with dexamethasone.

Consequently, antibodies of the present invention have an anti-inflammatory impact by decreasing neutrophils counts, reversing air-blood barrier damage and decreasing TNF alpha and KC-GRO cytokine levels in plasma.

What is claimed is:

1. An anti-Galectin-3 (anti-Gal3) antibody or an antigen-binding fragment thereof, wherein the antibody binds specifically to Galectin-3 and not to Galectin-1 or Galectin-7.

2. The anti-Gal3 antibody or antigen-binding fragment of claim 1, wherein the anti-Gal3 antibody or antigen-binding fragment of the invention binds to extracellular Gal3.

3. The anti-Gal3 antibody or antigen-binding fragment of claim 1 or 2, wherein the anti-Gal3 antibody or antigen-binding fragment of the invention inhibits Gal3 binding to its ligands or is an antagonist of Gal3.

4. The anti-Gal3 antibody or antigen-binding fragment of any one of claim 1 to 3, wherein the anti-Gal3 antibody comprises:
a heavy chain complementarity-determining region (H-CDR):
a) HCDR-1 comprising or consisting of the amino acid sequence of any one of SEQ ID NO: 1-5,
b) (H-CDR) 2 comprising or consisting of the amino acid sequence of any one of SEQ ID NO: 6-11, and
c) (H-CDR) 3 comprising or consisting of the amino acid sequence of any one of SEQ ID NO: 12-19, and
a light chain complementarity-determining region (L-CDR):
a) (L-CDR) 1 comprising or consisting of the amino acid sequence of any one of SEQ ID NO: 20-26.
b) (L-CDR) 2 comprising or consisting of the amino acid sequence of any one of SEQ ID NO: 27-29, and
c) (L-CDR) 3 comprising or consisting of the amino acid sequence of any one of SEQ ID NO: 30-37.

5. The anti-Gal3 antibody or antigen-binding fragment of any one of claim 1 to 4, wherein the anti-Gal3 antibody comprises the H-CDR 1-3 and L-CDR 1-3 comprising or consisting of the amino acid sequences of:
a) SEQ ID NOs: 1, 6, 12, 20, 27 and 30, respectively;
b) SEQ ID NOs: 1, 6, 13, 21, 27 and 31, respectively;
c) SEQ ID NOs: 1, 6, 14, 22, 27 and 31, respectively;
d) SEQ ID NOs: 5, 7, 3, 21, 28 and 32, respectively;
e) SEQ ID NOs: 3, 8, 15, 23, 27 and 33, respectively;
f) SEQ ID NOs: 2, 6, 16, 24, 27 and 34, respectively;
g) SEQ ID NOs: 1, 9, 14, 24, 27 and 34, respectively;
h) SEQ ID NOs: 2, 10, 17, 25, 27 and 35, respectively;
i) SEQ ID NOs: 4, 11, 18, 21, 29 and 36, respectively; or
j) SEQ ID NOs: 2, 10, 19, 26, 27 and 37, respectively;

6. The anti-Gal3 antibody or antigen-binding fragment of any one of claim 1 to 5, wherein the anti-Gal3 antibody comprises:
a) a heavy chain variable domain VH that is at least 90% identical in sequence to the VH amino acid sequence of SEQ ID NO: 38, 40, 42, 44, 46, 48, 50, 52, 54, 56, 81, 82, 84, 85, 87, 88, 90, 92, 94, 95, 97, 99, 101, 103 or 104, and
b) a light chain variable domain VL that is at least 90% identical in sequence to the VL amino acid sequence of SEQ ID NO: 39, 41, 43, 45, 47, 49, 51, 53, 55, 57, 83, 86, 89, 91, 93, 96, 98, 100, 102 or 105.

7. The anti-Gal3 antibody or antigen-binding fragment of any one of claim 1 to 6, wherein
a) the heavy chain variable domain VH comprises or consists of SEQ ID NO: 38, 40, 42, 44, 46, 48, 50, 52, 54, 56, 81, 82, 84, 85, 87, 88, 90, 92, 94, 95, 97, 99,101,103 or 104, and
b) a light chain variable domain VL comprises or consists of SEQ ID NO: 39, 41, 43, 45, 47, 49, 51, 53, 55, 57, 83, 86, 89, 91, 93, 96, 98, 100, 102 or 105.

8. The anti-Gal3 antibody or antigen-binding fragment of any one of claim 1 to 7, wherein the anti-Gal3 antibody or antigen-binding fragment of the invention:
a) has a VH that comprises or consists of the amino acid sequence of SEQ ID NO: 38, and a VL that comprises or consists of the amino acid sequence of SEQ ID NO: 39;
b) has a VH that comprises or consists of the amino acid sequence of SEQ ID NO: 40, and a VL that comprises or consists of the amino acid sequence of SEQ ID NO: 41;
c) has a VH that comprises or consists of the amino acid sequence of SEQ ID NO: 42, and a VL that comprises or consists of the amino acid sequence of SEQ ID NO: 43;
d) has a VH that comprises or consists of the amino acid sequence of SEQ ID NO: 44, and a VL that comprises or consists of the amino acid sequence of SEQ ID NO: 45;
e) has a VH that comprises or consists of the amino acid sequence of SEQ ID NO: 46, and a VL that comprises or consists of the amino acid sequence of SEQ ID NO: 47;
f) has a VH that comprises or consists of the amino acid sequence of SEQ ID NO: 48, and a VL that comprises or consists of the amino acid sequence of SEQ ID NO: 49;
g) has a VH that comprises or consists of the amino acid sequence of SEQ ID NO: 50, and a VL that comprises or consists of the amino acid sequence of SEQ ID NO: 51;
h) has a VH that comprises or consists of the amino acid sequence of SEQ ID NO: 52, and a VL that comprises or consists of the amino acid sequence of SEQ ID NO: 53;
i) has a VH that comprises or consists of the amino acid sequence of SEQ ID NO: 54, and a VL that comprises or consists of the amino acid sequence of SEQ ID NO: 55;
j) has a VH that comprises or consists of the amino acid sequence of SEQ ID NO: 56, and a VL that comprises or consists of the amino acid sequence of SEQ ID NO: 57;
k) has a VH that comprises or consists of the amino acid sequence of SEQ ID NO: 81, and a VL that comprises or consists of the amino acid sequence of SEQ ID NO: 83;
l) has a VH that comprises or consists of the amino acid sequence of SEQ ID NO: 82, and a VL that comprises or consists of the amino acid sequence of SEQ ID NO: 83;
m) has a VH that comprises or consists of the amino acid sequence of SEQ ID NO: 84, and a VL that comprises or consists of the amino acid sequence of SEQ ID NO: 86;
n) has a VH that comprises or consists of the amino acid sequence of SEQ ID NO: 85, and a VL that comprises or consists of the amino acid sequence of SEQ ID NO: 86;
o) has a VH that comprises or consists of the amino acid sequence of SEQ ID NO: 87, and a VL that comprises or consists of the amino acid sequence of SEQ ID NO: 89;
p) has a VH that comprises or consists of the amino acid sequence of SEQ ID NO: 88, and a VL that comprises or consists of the amino acid sequence of SEQ ID NO: 89;
q) has a VH that comprises or consists of the amino acid sequence of SEQ ID NO: 90, and a VL that comprises or consists of the amino acid sequence of SEQ ID NO: 91;
r) has a VH that comprises or consists of the amino acid sequence of SEQ ID NO: 92, and a VL that comprises or consists of the amino acid sequence of SEQ ID NO: 93;
s) has a VH that comprises or consists of the amino acid sequence of SEQ ID NO: 94, and a VL that comprises or consists of the amino acid sequence of SEQ ID NO: 96;
t) has a VH that comprises or consists of the amino acid sequence of SEQ ID NO: 95, and a VL that comprises or consists of the amino acid sequence of SEQ ID NO: 96;
u) has a VH that comprises or consists of the amino acid sequence of SEQ ID NO: 97, and a VL that comprises or consists of the amino acid sequence of SEQ ID NO: 98;
v) has a VH that comprises or consists of the amino acid sequence of SEQ ID NO: 99, and a VL that comprises or consists of the amino acid sequence of SEQ ID NO: 100;
w) has a VH that comprises or consists of the amino acid sequence of SEQ ID NO: 101, and a VL that comprises or consists of the amino acid sequence of SEQ ID NO: 102;
x) has a VH that comprises or consists of the amino acid sequence of SEQ ID NO: 103, and a VL that comprises or consists of the amino acid sequence of SEQ ID NO: 105; or
y) has a VH that comprises or consists of the amino acid sequence of SEQ ID NO: 104, and a VL that comprises or consists of the amino acid sequence of SEQ ID NO: 105.

9. The anti-Gal3 antibody or antigen-binding fragment of any one of claims 1-8, wherein the antibody competes for binding to human Gal3 with antibody D06, D11, E01, E02, E07, G03, H07, H10, B12, E12, D06-G1, D06-G2, D11-G1, D11-G2, E01-G, E02-G, E07-G1, E07-G2, G03-G1, G03-G2, H07-G, H10-G, B12-G, E12-G1 or E12-G2.

10. The anti-Gal3 antibody of any one of claims 1-9, wherein the antibody is of isotype IgG.

11. The anti-Gal3 antibody of claim 10, wherein the antibody is of isotype IgG subclass IgG1 or IgG4.

12. The anti-Gal3 antibody of claim 11, wherein the antibody comprises at least one mutation in the Fc region.

13. The anti-Gal3 antibody of claim 12, wherein the antibody comprises a mutation in one or more of heavy chain amino acid positions 228, 234 and 235, which are numbered according to the EU numbering scheme.

14. The anti-Gal3 antibody of claim 13, wherein one or both of the amino acid residues at positions 234 and 235 are mutated from Leu to Ala, and/or wherein the amino acid residue at position 228 is mutated from Ser to Pro.

15. A pharmaceutical composition comprising at least one anti-Gal3 antibody or antigen-binding fragment thereof according to any one of claims 1-14 and a pharmaceutically acceptable excipient.

16. An isolated nucleic acid molecule comprising a nucleotide sequence that encodes the heavy chain or an antigen-binding fragment thereof, or a nucleotide sequence that encodes the light chain or an antigen-binding fragment thereof, or both, of the anti-Gal3 antibody of any one of claims 1-14.

17. A combination of isolated nucleic acid molecules encoding an anti-Gal3 antibody or antigen-binding fragment thereof according to any one of claims 1-14, in particular a first isolated nucleic acid molecule comprising or consisting of a sequence encoding a VH sequence and a second isolated nucleic acid molecule comprising or consisting of a sequence encoding a VL sequence.

18. A vector comprising the isolated nucleic acid molecule according to claim 16 or the combination of isolated nucleic acid molecules according to claim 17, wherein said vector further comprises an expression control sequence.

19. A host cell comprising the isolated nucleic acid molecule according to claim 16 or the combination of isolated nucleic acid molecules according to claim 17 or comprising the vector according to claim 18.

20. A method for producing an anti-Gal3 antibody or an antigen-binding fragment thereof, comprising providing a host cell according to claim 19, culturing said host cell under conditions suitable for expression of the antibody or fragment, and isolating the resulting antibody or fragment.

21. A bi-specific binding molecule comprising an anti-Gal3 antibody or antigen-binding fragment thereof according to any one of claims 1-14.

22. A method for treating a patient with a Gal3-related disease, comprising administering to said patient the anti-Gal3 antibody or antigen-binding fragment according to any one of claims 1-14, a pharmaceutical composition according to claim 15, or a bi-specific binding molecule according to claim 21.

23. The method according to claim 22, wherein the Gal3 related disease is a fibrotic disease, inflammatory diseases, autoimmune disease, immune-mediated disorder, neurodegenerative disease, metabolic disease, infectious disease or cancer.

24. The method of claim 23, wherein the fibrotic disease originates in a tissue selected from skin, lung, liver, heart, kidney and vessels.

25. The method of any one of claims 22-24, further comprising administering to the patient a further additional therapeutic agent.

26. The method of claim 25, wherein the further additional therapeutic agent is an immune checkpoint modulator, a chemotherapeutic agent, anti-neoplastic agent, anti-angiogenic agent, hormonal therapeutic agent, stem cell-based therapeutic agent, surgery, radiation therapy, another inhibitor of the Gal3 pathway or which is administered in PD1/PDL1 blockade therapies and/or a CTLA4 blockade therapy.

27. Use of an anti-Gal3 antibody or antigen-binding fragment according to any one of claims 1-14 for the manufacture of a medicament for:
a) treating a patient with a Gal3-related disease; or
b) treating fibrotic disease, inflammatory diseases, autoimmune disease, immune-mediated disorder, neurodegenerative disease, metabolic disease, infectious disease or cancer in a patient; or
c) inhibiting Gal3 interactions in a patient in need thereof.

28. An antibody or antigen-binding fragment of any one of claims 1-14 for use in:
a) treating a patient with a Gal3-related disease; or
b) treating fibrotic disease, inflammatory diseases, autoimmune disease, immune-mediated disorder, neurodegenerative disease, metabolic disease, infectious disease or cancer in a patient; or
c) inhibiting Gal3 interactions in a patient in need thereof.

29. An article of manufacture comprising the antibody or antigen-binding fragment of any one of claims 1-14, wherein said article of manufacture is suitable for:
a) treating a patient with a Gal3-related disease; or
b) treating fibrotic disease, inflammatory diseases, autoimmune disease, immune-mediated disorder, neurodegenerative disease, metabolic disease, infectious disease or cancer in a patient; or
c) inhibiting Gal3 interactions in a patient in need thereof.


SEQUENCE LISTING (110 sequences)

SEQ ID NO: 1 - protein, 8 aa, synthetic construct
GFTFSNYN

SEQ ID NO: 2 - protein, 8 aa, synthetic construct
GFTFSNYA

SEQ ID NO: 3 - protein, 8 aa, synthetic construct
GFTFSNYY

SEQ ID NO: 4 - protein, 8 aa, synthetic construct
GFTFSNDD

SEQ ID NO: 5 - protein, 16 aa, synthetic construct
VRSSYGYSNYGSGMDV

SEQ ID NO: 6 - protein, 8 aa, synthetic construct
IYGSSRYI

SEQ ID NO: 7 - protein, 8 aa, synthetic construct
INGASRDI

SEQ ID NO: 8 - protein, 8 aa, synthetic construct
INGASRNI

SEQ ID NO: 9 - protein, 8 aa, synthetic construct
IDGSSRYI

SEQ ID NO: 10 - protein, 8 aa, synthetic construct
IYGSSRSI

SEQ ID NO: 11 - protein, 8 aa, synthetic construct
IDGSSRSI

SEQ ID NO: 12 - protein, 10 aa, synthetic construct
VRSGYGGMDV

SEQ ID NO: 13 - protein, 10 aa, synthetic construct
VRSSYGGMDV

SEQ ID NO: 14 - protein, 10 aa, synthetic construct
VRSYYGGMDV

SEQ ID NO: 15 - protein, 12 aa, synthetic construct
VRSSDYDGGMDV

SEQ ID NO: 16 - protein, 10 aa, synthetic construct
VRSYNGGMDV

SEQ ID NO: 17 - protein, 15 aa, synthetic construct
VRSSYNYYYGNGMDV

SEQ ID NO: 18 - protein, 12 aa, synthetic construct
VRSSGYSGGGMD

SEQ ID NO: 19 - protein, 10 aa, synthetic construct
VRSNNAGMDV

SEQ ID NO: 20 - protein, 9 aa, synthetic construct
SSDVGGYNG

SEQ ID NO: 21 - protein, 9 aa, synthetic construct
SSDVGGSYN

SEQ ID NO: 22 - protein, 9 aa, synthetic construct
SSDVGGYYS

SEQ ID NO: 23 - protein, 9 aa, synthetic construct
SSDVGGNSY

SEQ ID NO: 24 - protein, 9 aa, synthetic construct
SSDVGGYYN

SEQ ID NO: 25 - protein, 9 aa, synthetic construct
SSDVGGYGG

SEQ ID NO: 26 - protein, 9 aa, synthetic construct
SSDVGGYYD

SEQ ID NO: 27-29 - skipped (3 entries)

SEQ ID NO: 30 - protein, 10 aa, synthetic construct
SSGTDYSTRV

SEQ ID NO: 31 - protein, 10 aa, synthetic construct
SSYTGYSTRV

SEQ ID NO: 32 - protein, 10 aa, synthetic construct
SSNTADSTRV

SEQ ID NO: 33 - protein, 9 aa, synthetic construct
SSSTYGSTR

SEQ ID NO: 34 - protein, 10 aa, synthetic construct
SSSTGYSTRV

SEQ ID NO: 35 - protein, 9 aa, synthetic construct
SSSTSYSTR

SEQ ID NO: 36 - protein, 9 aa, synthetic construct
SSYTSNSAR

SEQ ID NO: 37 - protein, 9 aa, synthetic construct
SSYTSYSTR

SEQ ID NO: 38 - protein, 117 aa, synthetic construct
EVQLVESGGSLVKPGGSLRLSCAASGFTFSNYNMNWVRQAPGKGLEWISSIYGSSRYIYYADFVKGRFTISRDNAKNSLYLQMNSLRAEDTAVYYCVRSGYGGMDVWGRGTLVTVSS

SEQ ID NO: 39 - protein, 110 aa, synthetic construct
QSVLTQPASVSGSPGQSITISCAGTSSDVGGYNGVSWYQQHPGKAPKLMIYYDSYRPSGVSNRFSGSKSGNTASLTISGLQAEDEADYYCSSGTDYSTRVFGGGTKLEIK

SEQ ID NO: 40 - protein, 117 aa, synthetic construct
EVQLVESGGSLVKPGGSLRLSCAASGFTFSNYNMNWVRQAPGKGLEWISSIYGSSRYIYYADFVKGRFTISRDNAKNSLYLQMNSLRAEDTAVYYCVRSSYGGMDVWGRGTLVTVSS

SEQ ID NO: 41 - protein, 110 aa, synthetic construct
QSVLTQPASVSGSPGQSITISCAGTSSDVGGSYNVAWYQQHPGKAPKLMIYYDSYRPSGVSNRFSGSKSGNTASLTISGLQAEDEADYYCSSYTGYSTRVFGGGTKLEIK

SEQ ID NO: 42 - protein, 117 aa, synthetic construct
EVQLVESGGSLVKPGGSLRLSCAASGFTFSNYNMNWVRQAPGKGLEWISNIYGSSRYIYYADFVKGRFTISRDNAKNSLYLQMNSLRAEDTAVYYCVRSYYGGMDVWGRGTLVTVSS

SEQ ID NO: 43 - protein, 110 aa, synthetic construct
QSVLTQPASVSGSPGQSITISCAGTSSDVGGYYSVSWYQQHPGKAPKLMIYYDSNRPSGVSNRFSGSKSGNTASLTISGLQAEDEADYYCSSYTGYSTRVFGGGTKLEIK

SEQ ID NO: 44 - protein, 123 aa, synthetic construct
EVQLVESGGSLVKPGGSLRLSCAASGFTFSNYYMNWVRQAPGKGLEWISYINGASRDISYADFVKGRFTISRDNAKNSLYLQMNSLRAEDTAVYYCVRSSYGYSNYGSGMDVWGRGTLVTVSS

SEQ ID NO: 45 - protein, 110 aa, synthetic construct
QSVLTQPASVSGSPGQSITISCAGTSSDVGGSYNVAWYQQHPGKAPKLMIYNDANRPSGVSNRFSGSKSGNTASLTISGLQAEDEADYYCSSNTADSTRVFGGGTKLEIK

SEQ ID NO: 46 - protein, 119 aa, synthetic construct
EVQLVESGGSLVKPGGSLRLSCAASGFTFSNNYMNWVRQAPGKGLEWISNINGASRNIAYADFVKGRFTISRDNAKNSLYLQMNSLRAEDTAVYYCVRSSDYDGGMDVWGRGTLVTVSS

SEQ ID NO: 47 - protein, 110 aa, synthetic construct
QSVLTQPASVSGSPGQSITISCAGTSSDVGGNSYVSWYQQHPGKAPKLMIYYDSYRPSGVSNRFSGSKSGNTASLTISGLQAEDEADYYCSSSTYGSTRVFGGGTKLEIK

SEQ ID NO: 48 - protein, 117 aa, synthetic construct
EVQLVESGGSLVKPGGSLRLSCAASGFTFSNYAMNWVRQAPGKGLEWISSIYGSSRYIYYADFVKGRFTISRDNAKNSLYLQMNSLRAEDTAVYYCVRSYNGGMDVWGRGTLVTVSS

SEQ ID NO: 49 - protein, 110 aa, synthetic construct
QSVLTQPASVSGSPGQSITISCAGTSSDVGGYYNVAWYQQHPGKAPKLMIYYDSYRPSGVSNRFSGSKSGNTASLTISGLQAEDEADYYCSSSTGYSTRVFGGGTKLEIK

SEQ ID NO: 50 - protein, 117 aa, synthetic construct
EVQLVESGGSLVKPGGSLRLSCAASGFTFSNYNMNWVRQAPGKGLEWISNIDGSSRYIYYADFVKGRFTISRDNAKNSLYLQMNSLRAEDTAVYYCVRSYYGGMDVWGRGTLVTVSS

SEQ ID NO: 51 - protein, 110 aa, synthetic construct; identical to SEQ ID NO: 49

SEQ ID NO: 52 - protein, 122 aa, synthetic construct
EVQLVESGGSLVKPGGSLRLSCAASGFTFSNYAMNWVRQAPGKGLEWISSIYGSSRSISYADFVKGRFTISRDNAKNSLYLQMNSLRAEDTAVYYCVRSSYNYYYGNGMDVWGRGTLVTVSS

SEQ ID NO: 53 - protein, 110 aa, synthetic construct
QSVLTQPASVSGSPGQSITISCAGTSSDVGGYGGVSWYQQHPGKAPKLMIYYDSYRPSGVSNRFSGSKSGNTASLTISGLQAEDEADYYCSSSTSYSTRVFGGGTKLEIK

SEQ ID NO: 54 - protein, 120 aa, synthetic construct
EVQLVESGGSLVKPGGSLRLSCAASGFTFSNDDMNWVRQAPGKGLEWISSIDGSSRSIYYADFVKGRFTISRDNAKNSLYLQMNSLRAEDTAVYYCVRSSGYSGGGMDVWGRGTLVTVSS

SEQ ID NO: 55 - protein, 110 aa, synthetic construct
QSVLTQPASVSGSPGQSITISCAGTSSDVGGSYNVAWYQQHPGKAPKLMIYSDSYRPSGVSNRFSGSKSGNTASLTISGLQAEDEADYYCSSYTSNSARVFGGGTKLEIK

SEQ ID NO: 56 - protein, 117 aa, synthetic construct
EVQLVESGGSLVKPGGSLRLSCAASGFTFSNYAMNWVRQAPGKGLEWISSIYGSSRSIYYADFVKGRFTISRDNAKNSLYLQMNSLRAEDTAVYYCVRSNNAGMDVWGRGTLVTVSS

SEQ ID NO: 57 - protein, 110 aa, synthetic construct
QSVLTQPASVSGSPGQSITISCAGTSSDVGGYYDVSWYQQHPGKAPKLMIYYDSYRPSGVSNRFSGSKSGNTASLTISGLQAEDEADYYCSSYTSYSTRVFGGGTKLEIK

SEQ ID NO: 58 - protein, 330 aa, synthetic construct [sequence omitted]

SEQ ID NO: 59 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 107 aa, synthetic construct [sequence omitted]

SEQ ID NO: 61 - DNA, 1356 bp, synthetic construct
  source 1..1356: mol_type = other DNA
gaggtccaactggtcgagagcgggggatcgctggtcaagccggggggaagccttagactatcatgcgccgcgagcggatttacatttagcaactacaatatgaactgggtcagacaagcaccgggaaaggggctggaatggatatcaagcatttatggaagctcgcgatatatttactatgcggactttgtgaaggggagattcactatatcgcgcgacaatgcgaaaaatagcctatacctgcaaatgaactcattgagggctgaggacacggcggtctattattgcgttcgaagcggatatggaggcatggatgtatggggaagagggactctggtcacggtctcgagcgctagcacaaagggccctagtgtgtttcctctggctccctcttccaaatccacttctggtggcactgctgctctgggatgcctggtgaaggattactttcctgaacctgtgactgtctcatggaactctggtgctctgacttctggtgtccacactttccctgctgtgctgcagtctagtggactgtactctctgtcatctgtggtcactgtgccctcttcatctctgggaacccagacctacatttgtaatgtgaaccacaaaccatccaacactaaagtggacaaaaaagtggaacccaaatcctgtgacaaaacccacacctgcccaccttgtcctgcccctgaagccgccggaggaccttctgtgtttctgttcccccccaaaccaaaggataccctgatgatctctagaacccctgaggtgacatgtgtggtggtggatgtgtctcatgaggaccctgaggtcaaattcaactggtacgtggatggagtggaagtccacaatgccaaaaccaagcctagagaggaacagtacaattcaacctacagagtggtcagtgtgctgactgtgctgcatcaggattggctgaatggcaaggaatacaagtgtaaagtctcaaacaaggccctgcctgctccaattgagaaaacaatctcaaaggccaagggacagcctagggaaccccaggtctacaccctgccaccttcaagagacgaactgaccaaaaaccaggtgtccctgacatgcctggtcaaaggcttctacccttctgacattgctgtggagtgggagtcaaatggacagcctgagaacaactacaaaacaaccccccctgtgctggattctgatggctctttctttctgtactccaaactgactgtggacaagtctagatggcagcaggggaatgtcttttcttgctctgtcatgcatgaggctctgcataaccactacactcagaaatccctgtctctgtctcccgggaaatgatagtaaaagctt

SEQ ID NO: 62 - DNA, 666 bp, synthetic construct
  source 1..666: mol_type = other DNA
caaagcgtcctgacgcaaccggcatcggtcagcgggagtccgggacaatcgattacaataagctgcgcggggactagctcggacgtcgggggatataatggcgtctcatggtatcaacaacaccccggaaaagctcctaaactgatgatttattacgattcctaccggccatcgggggtctcgaatcgatttagcggaagcaagagcgggaatacggcatcgctgacaatctccgggctgcaagccgaggatgaggccgattactactgttcgagcgggacggactattcgacgagagtatttggaggaggtaccaaactcgagatcaaacgtacggtcgcggcgccttctgtgttcattttccccccatctgatgaacagctgaaatctggcactgcttctgtggtctgtctgctgaacaacttctaccctagagaggccaaagtccagtggaaagtggacaatgctctgcagagtgggaattcccaggaatctgtcactgagcaggactctaaggatagcacatactccctgtcctctactctgacactgagcaaggctgattacgagaaacacaaagtgtacgcctgtgaagtcacacatcaggggctgtctagtcctgtgaccaaatccttcaataggggagagtgctgatagtaaaagctt

SEQ ID NO: 63 - DNA, 1356 bp, synthetic construct
  source 1..1356: mol_type = other DNA
gaggtacaactggtcgaatcgggcggctcgctggtgaagccgggagggagcttgcggctatcatgcgcggcctcgggatttacattctcgaactataatatgaactgggtccggcaagctccgggaaaggggcttgaatggatttcgagcatatacggatcaagccgatatatatactacgcggattttgtgaaagggcgattcacaatatcacgggataacgcgaagaatagcctatacctgcaaatgaactctctgagggccgaggataccgcggtatattattgcgtgcggagctcatacggaggcatggacgtatggggacgggggacgctcgtgacggtctcgagcgctagcacaaagggccctagtgtgtttcctctggctccctcttccaaatccacttctggtggcactgctgctctgggatgcctggtgaaggattactttcctgaacctgtgactgtctcatggaactctggtgctctgacttctggtgtccacactttccctgctgtgctgcagtctagtggactgtactctctgtcatctgtggtcactgtgccctcttcatctctgggaacccagacctacatttgtaatgtgaaccacaaaccatccaacactaaagtggacaaaaaagtggaacccaaatcctgtgacaaaacccacacctgcccaccttgtcctgcccctgaagccgccggaggaccttctgtgtttctgttcccccccaaaccaaaggataccctgatgatctctagaacccctgaggtgacatgtgtggtggtggatgtgtctcatgaggaccctgaggtcaaattcaactggtacgtggatggagtggaagtccacaatgccaaaaccaagcctagagaggaacagtacaattcaacctacagagtggtcagtgtgctgactgtgctgcatcaggattggctgaatggcaaggaatacaagtgtaaagtctcaaacaaggccctgcctgctccaattgagaaaacaatctcaaaggccaagggacagcctagggaaccccaggtctacaccctgccaccttcaagagacgaactgaccaaaaaccaggtgtccctgacatgcctggtcaaaggcttctacccttctgacattgctgtggagtgggagtcaaatggacagcctgagaacaactacaaaacaaccccccctgtgctggattctgatggctctttctttctgtactccaaactgactgtggacaagtctagatggcagcaggggaatgtcttttcttgctctgtcatgcatgaggctctgcataaccactacactcagaaatccctgtctctgtctcccgggaaatgatagtaaaagctt

SEQ ID NO: 64 - DNA, 666 bp, synthetic construct
  source 1..666: mol_type = other DNA
caaagcgttctgacgcaaccggcgagcgtcagcggatcacccggccaatccataacgataagctgcgccgggacatcatccgatgtggggggatcatataacgtcgcatggtatcaacaacatcctgggaaggctccaaaattaatgatttattacgactcataccggccaagcggcgtttcaaatagatttagcggctccaagagcgggaatacggcaagcctgacgataagcgggctacaagcggaggatgaggctgactattattgctcatcatataccggatactcgactagagtattcggaggaggtaccaaactcgagatcaaacgtacggtcgcggcgccttctgtgttcattttccccccatctgatgaacagctgaaatctggcactgcttctgtggtctgtctgctgaacaacttctaccctagagaggccaaagtccagtggaaagtggacaatgctctgcagagtgggaattcccaggaatctgtcactgagcaggactctaaggatagcacatactccctgtcctctactctgacactgagcaaggctgattacgagaaacacaaagtgtacgcctgtgaagtcacacatcaggggctgtctagtcctgtgaccaaatccttcaataggggagagtgctgatagtaaaagctt

SEQ ID NO: 65 - DNA, 1356 bp, synthetic construct
  source 1..1356: mol_type = other DNA
gaggtacaactagtagaatcggggggaagcctggttaagcccggaggatcgctgcggcttagctgcgcggcatccggctttacatttagcaactacaatatgaattgggtccggcaagcgcctgggaaaggactggaatggataagcaatatctacgggagcagcagatatatatactacgcggatttcgtgaaagggcgattcacgatctcgcgggataacgcgaagaactcgctatacctgcaaatgaactctctgcgcgccgaggataccgcggtttactactgcgtgcgatcatactacggaggcatggacgtatggggccgggggacactagtgaccgtctcgagcgctagcacaaagggccctagtgtgtttcctctggctccctcttccaaatccacttctggtggcactgctgctctgggatgcctggtgaaggattactttcctgaacctgtgactgtctcatggaactctggtgctctgacttctggtgtccacactttccctgctgtgctgcagtctagtggactgtactctctgtcatctgtggtcactgtgccctcttcatctctgggaacccagacctacatttgtaatgtgaaccacaaaccatccaacactaaagtggacaaaaaagtggaacccaaatcctgtgacaaaacccacacctgcccaccttgtcctgcccctgaagccgccggaggaccttctgtgtttctgttcccccccaaaccaaaggataccctgatgatctctagaacccctgaggtgacatgtgtggtggtggatgtgtctcatgaggaccctgaggtcaaattcaactggtacgtggatggagtggaagtccacaatgccaaaaccaagcctagagaggaacagtacaattcaacctacagagtggtcagtgtgctgactgtgctgcatcaggattggctgaatggcaaggaatacaagtgtaaagtctcaaacaaggccctgcctgctccaattgagaaaacaatctcaaaggccaagggacagcctagggaaccccaggtctacaccctgccaccttcaagagacgaactgaccaaaaaccaggtgtccctgacatgcctggtcaaaggcttctacccttctgacattgctgtggagtgggagtcaaatggacagcctgagaacaactacaaaacaaccccccctgtgctggattctgatggctctttctttctgtactccaaactgactgtggacaagtctagatggcagcaggggaatgtcttttcttgctctgtcatgcatgaggctctgcataaccactacactcagaaatccctgtctctgtctcccgggaaatgatagtaaaagctt

SEQ ID NO: 66 - DNA, 666 bp, synthetic construct
  source 1..666: mol_type = other DNA
caaagcgtcctgacacaaccggcatccgtcagcgggagtccggggcaaagcatcacgattagctgcgccgggacatcaagcgatgtggggggatactactcggtctcatggtatcaacaacatccgggaaaggcaccgaaattgatgatatattatgactccaaccggccatcgggggtctccaatagattttcgggaagcaaatcggggaacaccgcgagcctgacaatttcgggactacaagccgaggatgaggcggactattattgctcatcatatacgggatactctacgagggtctttggaggaggtaccaaactcgagatcaaacgtacggtcgcggcgccttctgtgttcattttccccccatctgatgaacagctgaaatctggcactgcttctgtggtctgtctgctgaacaacttctaccctagagaggccaaagtccagtggaaagtggacaatgctctgcagagtgggaattcccaggaatctgtcactgagcaggactctaaggatagcacatactccctgtcctctactctgacactgagcaaggctgattacgagaaacacaaagtgtacgcctgtgaagtcacacatcaggggctgtctagtcctgtgaccaaatccttcaataggggagagtgctgatagtaaaagctt

SEQ ID NO: 67 - DNA, 1374 bp, synthetic construct
  source 1..1374: mol_type = other DNA
gaggtccaactagtcgaatcggggggatcgctcgtcaagcctggggggagcctgagactatcatgcgcggcctccggcttcacatttagcaactactacatgaactgggtcaggcaagcaccgggaaaggggctggaatggatatcatatattaatggggcaagccgggatatctcatacgccgacttcgtcaaggggcgattcacgatatcaagggacaatgcgaagaatagcctatacctgcaaatgaatagcctgagggccgaggacacggcggtttactactgcgtcagatcatcatacggatattcgaactacggatcggggatggatgtatggggacgcggaacattggtcacggtctcgagcgctagcacaaagggccctagtgtgtttcctctggctccctcttccaaatccacttctggtggcactgctgctctgggatgcctggtgaaggattactttcctgaacctgtgactgtctcatggaactctggtgctctgacttctggtgtccacactttccctgctgtgctgcagtctagtggactgtactctctgtcatctgtggtcactgtgccctcttcatctctgggaacccagacctacatttgtaatgtgaaccacaaaccatccaacactaaagtggacaaaaaagtggaacccaaatcctgtgacaaaacccacacctgcccaccttgtcctgcccctgaagccgccggaggaccttctgtgtttctgttcccccccaaaccaaaggataccctgatgatctctagaacccctgaggtgacatgtgtggtggtggatgtgtctcatgaggaccctgaggtcaaattcaactggtacgtggatggagtggaagtccacaatgccaaaaccaagcctagagaggaacagtacaattcaacctacagagtggtcagtgtgctgactgtgctgcatcaggattggctgaatggcaaggaatacaagtgtaaagtctcaaacaaggccctgcctgctccaattgagaaaacaatctcaaaggccaagggacagcctagggaaccccaggtctacaccctgccaccttcaagagacgaactgaccaaaaaccaggtgtccctgacatgcctggtcaaaggcttctacccttctgacattgctgtggagtgggagtcaaatggacagcctgagaacaactacaaaacaaccccccctgtgctggattctgatggctctttctttctgtactccaaactgactgtggacaagtctagatggcagcaggggaatgtcttttcttgctctgtcatgcatgaggctctgcataaccactacactcagaaatccctgtctctgtctcccgggaaatgatagtaaaagctt

SEQ ID NO: 68 - DNA, 666 bp, synthetic construct
  source 1..666: mol_type = other DNA
caaagcgtcttaacccaaccggcatcggtctctggctcgccggggcaatcaatcacgatttcatgcgccggcacatcaagcgatgtaggggggagctataatgtcgcatggtatcaacaacatccggggaaagcaccgaagctcatgatatataacgacgcaaaccggccaagcggagtatcaaatcgattttcgggctccaagagcgggaatacggcaagcctgacaatctctgggctgcaagcggaggacgaggccgactactattgctcatcgaacaccgcggactcaacgcgagtatttggaggaggtaccaaactcgagatcaaacgtacggtcgcggcgccttctgtgttcattttccccccatctgatgaacagctgaaatctggcactgcttctgtggtctgtctgctgaacaacttctaccctagagaggccaaagtccagtggaaagtggacaatgctctgcagagtgggaattcccaggaatctgtcactgagcaggactctaaggatagcacatactccctgtcctctactctgacactgagcaaggctgattacgagaaacacaaagtgtacgcctgtgaagtcacacatcaggggctgtctagtcctgtgaccaaatccttcaataggggagagtgctgatagtaaaagctt

SEQ ID NO: 69 - DNA, 1362 bp, synthetic construct
  source 1..1362: mol_type = other DNA
gaggtgcagctggtggaatctgggggatctctagtaaaacccggaggatcgctacgcttatcttgcgctgcatctggatttacatttagcaataattacatgaactgggtccgacaagctccgggcaaggggctggaatggatctccaatatcaacggggcgagccgcaacatcgcatatgcggattttgtgaaagggagatttacgatctcgcgggacaacgcgaaaaattcattatacctgcaaatgaactcgctgcgagcggaggataccgccgtttattactgcgtgcggagcagcgattacgacggaggcatggacgtatgggggcggggcacgcttgtcacggtctcgagcgctagcacaaagggccctagtgtgtttcctctggctccctcttccaaatccacttctggtggcactgctgctctgggatgcctggtgaaggattactttcctgaacctgtgactgtctcatggaactctggtgctctgacttctggtgtccacactttccctgctgtgctgcagtctagtggactgtactctctgtcatctgtggtcactgtgccctcttcatctctgggaacccagacctacatttgtaatgtgaaccacaaaccatccaacactaaagtggacaaaaaagtggaacccaaatcctgtgacaaaacccacacctgcccaccttgtcctgcccctgaagccgccggaggaccttctgtgtttctgttcccccccaaaccaaaggataccctgatgatctctagaacccctgaggtgacatgtgtggtggtggatgtgtctcatgaggaccctgaggtcaaattcaactggtacgtggatggagtggaagtccacaatgccaaaaccaagcctagagaggaacagtacaattcaacctacagagtggtcagtgtgctgactgtgctgcatcaggattggctgaatggcaaggaatacaagtgtaaagtctcaaacaaggccctgcctgctccaattgagaaaacaatctcaaaggccaagggacagcctagggaaccccaggtctacaccctgccaccttcaagagacgaactgaccaaaaaccaggtgtccctgacatgcctggtcaaaggcttctacccttctgacattgctgtggagtgggagtcaaatggacagcctgagaacaactacaaaacaaccccccctgtgctggattctgatggctctttctttctgtactccaaactgactgtggacaagtctagatggcagcaggggaatgtcttttcttgctctgtcatgcatgaggctctgcataaccactacactcagaaatccctgtctctgtctcccgggaaatgatagtaaaagctt

SEQ ID NO: 70 - DNA, 666 bp, synthetic construct
  source 1..666: mol_type = other DNA
caatctgtgcttacacaacccgcatctgtgagcggaagtccggggcaatcgattactataagctgcgcgggcacgagctccgacgtcggagggaactcatatgtgagctggtatcaacaacatccggggaaggcaccgaagctgatgatatattacgatagctaccgaccatcgggcgtcagcaatagattctcgggatcgaaatccggcaatacggcaagcctgacaatttcggggctgcaagccgaggatgaggccgactactattgctcatcttcgacctatgggagcacgcgggtctttggaggaggtaccaaactcgagatcaaacgtacggtcgcggcgccttctgtgttcattttccccccatctgatgaacagctgaaatctggcactgcttctgtggtctgtctgctgaacaacttctaccctagagaggccaaagtccagtggaaagtggacaatgctctgcagagtgggaattcccaggaatctgtcactgagcaggactctaaggatagcacatactccctgtcctctactctgacactgagcaaggctgattacgagaaacacaaagtgtacgcctgtgaagtcacacatcaggggctgtctagtcctgtgaccaaatccttcaataggggagagtgctgatagtaaaagctt

SEQ ID NO: 71 - DNA, 1356 bp, synthetic construct
  source 1..1356: mol_type = other DNA
gaagtccaactggtcgaatccggcggatcgctcgtcaagccgggcggaagcctccggctatcatgcgcggcatcgggatttacatttagcaattacgcaatgaattgggtccggcaagcccccggcaaagggctggaatggatttcatcgatttatgggagctcgcgatatatctattacgcggattttgtgaaggggcgattcacgatctctagggacaacgcgaagaactcactatacctgcaaatgaactccttgagagccgaagacacggcggtctactattgcgtcaggagctacaatggaggcatggacgtctggggccggggcacgctcgtcacggtctcgagcgctagcacaaagggccctagtgtgtttcctctggctccctcttccaaatccacttctggtggcactgctgctctgggatgcctggtgaaggattactttcctgaacctgtgactgtctcatggaactctggtgctctgacttctggtgtccacactttccctgctgtgctgcagtctagtggactgtactctctgtcatctgtggtcactgtgccctcttcatctctgggaacccagacctacatttgtaatgtgaaccacaaaccatccaacactaaagtggacaaaaaagtggaacccaaatcctgtgacaaaacccacacctgcccaccttgtcctgcccctgaagccgccggaggaccttctgtgtttctgttcccccccaaaccaaaggataccctgatgatctctagaacccctgaggtgacatgtgtggtggtggatgtgtctcatgaggaccctgaggtcaaattcaactggtacgtggatggagtggaagtccacaatgccaaaaccaagcctagagaggaacagtacaattcaacctacagagtggtcagtgtgctgactgtgctgcatcaggattggctgaatggcaaggaatacaagtgtaaagtctcaaacaaggccctgcctgctccaattgagaaaacaatctcaaaggccaagggacagcctagggaaccccaggtctacaccctgccaccttcaagagacgaactgaccaaaaaccaggtgtccctgacatgcctggtcaaaggcttctacccttctgacattgctgtggagtgggagtcaaatggacagcctgagaacaactacaaaacaaccccccctgtgctggattctgatggctctttctttctgtactccaaactgactgtggacaagtctagatggcagcaggggaatgtcttttcttgctctgtcatgcatgaggctctgcataaccactacactcagaaatccctgtctctgtctcccgggaaatgatagtaaaagctt

SEQ ID NO: 72 - DNA, 666 bp, synthetic construct
  source 1..666: mol_type = other DNA
caatcggttctgacacaacccgcgagcgtctccggctcgccgggccaaagcattacgattagctgcgcggggactagctcggatgtggggggatactacaacgtcgcatggtatcaacaacatccggggaaggcacccaagctgatgatatactacgattcatataggccatcgggtgtcagcaatcgattttcggggagcaaatcggggaacacggcatcgctgacgatctccgggctgcaagccgaagatgaggcggattactactgctcatcgagcacgggatactctacaagggtcttcggaggaggtaccaaactcgagatcaaacgtacggtcgcggcgccttctgtgttcattttccccccatctgatgaacagctgaaatctggcactgcttctgtggtctgtctgctgaacaacttctaccctagagaggccaaagtccagtggaaagtggacaatgctctgcagagtgggaattcccaggaatctgtcactgagcaggactctaaggatagcacatactccctgtcctctactctgacactgagcaaggctgattacgagaaacacaaagtgtacgcctgtgaagtcacacatcaggggctgtctagtcctgtgaccaaatccttcaataggggagagtgctgatagtaaaagctt

SEQ ID NO: 73 - DNA, 1356 bp, synthetic construct
  source 1..1356: mol_type = other DNA
gaagtccaactcgtcgaatcgggaggatcattggtcaagccgggggggagcctgcggctatcatgcgccgcgagcggatttacatttagcaattacaatatgaactgggtccggcaagcccctgggaaggggctggaatggatttcgaacatagatgggagctcccgatatatttattatgctgactttgtgaaaggccgattcacgataagccgagataacgcgaagaactcactatatctgcaaatgaactcgctgagggcggaggataccgccgtctactactgcgtccgatcctattacggaggcatggacgtctgggggagggggactttggttacggtctcgagcgctagcacaaagggccctagtgtgtttcctctggctccctcttccaaatccacttctggtggcactgctgctctgggatgcctggtgaaggattactttcctgaacctgtgactgtctcatggaactctggtgctctgacttctggtgtccacactttccctgctgtgctgcagtctagtggactgtactctctgtcatctgtggtcactgtgccctcttcatctctgggaacccagacctacatttgtaatgtgaaccacaaaccatccaacactaaagtggacaaaaaagtggaacccaaatcctgtgacaaaacccacacctgcccaccttgtcctgcccctgaagccgccggaggaccttctgtgtttctgttcccccccaaaccaaaggataccctgatgatctctagaacccctgaggtgacatgtgtggtggtggatgtgtctcatgaggaccctgaggtcaaattcaactggtacgtggatggagtggaagtccacaatgccaaaaccaagcctagagaggaacagtacaattcaacctacagagtggtcagtgtgctgactgtgctgcatcaggattggctgaatggcaaggaatacaagtgtaaagtctcaaacaaggccctgcctgctccaattgagaaaacaatctcaaaggccaagggacagcctagggaaccccaggtctacaccctgccaccttcaagagacgaactgaccaaaaaccaggtgtccctgacatgcctggtcaaaggcttctacccttctgacattgctgtggagtgggagtcaaatggacagcctgagaacaactacaaaacaaccccccctgtgctggattctgatggctctttctttctgtactccaaactgactgtggacaagtctagatggcagcaggggaatgtcttttcttgctctgtcatgcatgaggctctgcataaccactacactcagaaatccctgtctctgtctcccgggaaatgatagtaaaagctt

SEQ ID NO: 74 - DNA, 666 bp, synthetic construct; identical to SEQ ID NO: 72
  source 1..666: mol_type = other DNA

SEQ ID NO: 75 - DNA, 1371 bp, synthetic construct
  source 1..1371: mol_type = other DNA
gaggtgcaattagtcgagagcggaggatcgctggtgaagccggggggaagcctgaggcttagctgcgccgcatcgggattcacatttagcaattatgcgatgaattgggtcagacaagcgccggggaagggcttggaatggatatcgagcatttacggatcatcacggagcatctcatatgctgatttcgtgaaggggcgatttacaatctcgcgggataacgcgaaaaactcactatacctgcaaatgaactcactgagggcggaggacaccgcggtatactattgcgttcgatcaagctacaactactactacgggaacgggatggatgtctgggggagaggaaccctggtaacggtctcgagcgctagcacaaagggccctagtgtgtttcctctggctccctcttccaaatccacttctggtggcactgctgctctgggatgcctggtgaaggattactttcctgaacctgtgactgtctcatggaactctggtgctctgacttctggtgtccacactttccctgctgtgctgcagtctagtggactgtactctctgtcatctgtggtcactgtgccctcttcatctctgggaacccagacctacatttgtaatgtgaaccacaaaccatccaacactaaagtggacaaaaaagtggaacccaaatcctgtgacaaaacccacacctgcccaccttgtcctgcccctgaagccgccggaggaccttctgtgtttctgttcccccccaaaccaaaggataccctgatgatctctagaacccctgaggtgacatgtgtggtggtggatgtgtctcatgaggaccctgaggtcaaattcaactggtacgtggatggagtggaagtccacaatgccaaaaccaagcctagagaggaacagtacaattcaacctacagagtggtcagtgtgctgactgtgctgcatcaggattggctgaatggcaaggaatacaagtgtaaagtctcaaacaaggccctgcctgctccaattgagaaaacaatctcaaaggccaagggacagcctagggaaccccaggtctacaccctgccaccttcaagagacgaactgaccaaaaaccaggtgtccctgacatgcctggtcaaaggcttctacccttctgacattgctgtggagtgggagtcaaatggacagcctgagaacaactacaaaacaaccccccctgtgctggattctgatggctctttctttctgtactccaaactgactgtggacaagtctagatggcagcaggggaatgtcttttcttgctctgtcatgcatgaggctctgcataaccactacactcagaaatccctgtctctgtctcccgggaaatgatagtaaaagctt

SEQ ID NO: 76 - DNA, 666 bp, synthetic construct
  source 1..666: mol_type = other DNA
caaagcgtcctgacgcaaccggcatctgtgagcgggagccctgggcaatcaataacaatctcatgcgcgggcacatcatcggacgtcgggggatatgggggcgtcagctggtatcaacaacaccctgggaaggctccaaagctgatgatttactacgactcataccggccgagcggggtctcaaatagatttagcggatcgaaaagcggaaataccgcgagcttgaccatatccggcctacaagccgaggacgaggccgactattattgctcatcatcaacgagctatagcacgagggtctttggaggaggtaccaaactcgagatcaaacgtacggtcgcggcgccttctgtgttcattttccccccatctgatgaacagctgaaatctggcactgcttctgtggtctgtctgctgaacaacttctaccctagagaggccaaagtccagtggaaagtggacaatgctctgcagagtgggaattcccaggaatctgtcactgagcaggactctaaggatagcacatactccctgtcctctactctgacactgagcaaggctgattacgagaaacacaaagtgtacgcctgtgaagtcacacatcaggggctgtctagtcctgtgaccaaatccttcaataggggagagtgctgatagtaaaagctt

SEQ ID NO: 77 - DNA, 1353 bp, synthetic construct
  source 1..1353: mol_type = other DNA
gaggtgcagctggtggagtctgggggaagcctggtcaagcctggggggtccctgagactctcctgtgcagcctctggattcaccttcagtaacgacgacatgaactgggtccgccaggctccagggaaggggctggagtggatctcatccattgacggtagtagtagaagcatatactacgcagacttcgtgaagggccgattcaccatctccagagacaacgccaagaactcactgtacctgcaaatgaacagcctgagagccgaggacacggctgtttattactgtgtgagatccagtggctacagcggcggtggtatggacgtctggggcagaggcaccctggtcaccgtctcctcagctagtaccaagggcccaagtgtctttcccctggcaccctcctccaagagcacctctgggggcacagcagccctgggctgcctggtgaaggactacttccccgaaccggtgacggtgtcatggaactcaggagccctgaccagcggcgtgcacaccttcccggctgtcttgcagtcctcaggactctactccctcagcagcgtggtgaccgtccccagcagctccttgggcacccagacctacatctgcaacgtgaatcacaagcccagcaacaccaaggtggacaagaaagttgagcccaaatcttgtgacaaaactcacacatgcccaccgtgcccagcacctgagctcctggggggaccgtcagtgttcctcttccccccaaaacccaaggacaccctcatgatctcccggacccccgaggtcacatgcgtggtggtggacgtgagccacgaggaccctgaagtgaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtcagcaacaaagccctcccagcccccatcgagaaaaccattagcaaagccaaagggcagccccgagaaccacaggtttacaccctgcccccatccagagatgagctgaccaagaaccaggtcagcctgacctgcctcgtgaaaggcttctatcccagcgacatcgccgtggagtgggagagcaatgggcagccggagaacaactacaagaccacgcctcccgtgctggactccgacggctccttcttcctctacagcaagctcaccgtggataagagcaggtggcagcaggggaacgtgttctcatgctccgtgatgcacgaggctctgcacaaccactacacgcagaagagcctctccctgtctccggggaaatga

SEQ ID NO: 78 - DNA, 654 bp, synthetic construct
  source 1..654: mol_type = other DNA
cagtctgtgctgactcagcctgcctccgtgtctgggagccctggacagtcgatcaccatctcctgcgctggaaccagcagtgacgttggtggtagctacaacgtcgcctggtaccaacaacacccaggcaaagcccccaaactcatgatttatagcgacagttaccggccctcaggggtttctaatcgcttctctggctccaagtctggcaacacggcctccctgacaatctctgggctccaggctgaggacgaggctgattattactgcagctcatatacaagcaacagcgcccgagttttcggcggagggaccaagctggagatcaaaagaactgtggctgccccatctgttttcatcttcccgccatctgatgagcagttgaaatctggaactgcctctgttgtgtgcctgctgaataacttctatcccagagaggccaaagtccagtggaaggtggataacgccctccaatctggtaactcccaggagagtgtcacagagcaggacagcaaggacagcacctacagcctcagcagcaccctgacgctgagcaaagcagactacgagaaacacaaagtgtacgcctgcgaagtcacccatcagggcctgagcagtcccgtcacaaagagcttcaacaggggagagtgttag

SEQ ID NO: 79 - DNA, 1344 bp, synthetic construct
  source 1..1344: mol_type = other DNA
gaggtgcagctggtggagtctgggggaagcctggtcaagcctggggggtccctgagactctcctgtgcagcctctggattcaccttcagtaactatgccatgaactgggtccgccaggctccagggaaggggctggagtggatctcatccatttacggtagtagtagaagcatatactacgcagacttcgtgaagggccgattcaccatctccagagacaacgccaagaactcactgtacctgcaaatgaacagcctgagagccgaggacacggctgtttattactgtgtgagatccaacaacgccggtatggacgtctggggcagaggcaccctggtcaccgtctcctcagctagtaccaagggcccaagtgtctttcccctggcaccctcctccaagagcacctctgggggcacagcagccctgggctgcctggtgaaggactacttccccgaaccggtgacggtgtcatggaactcaggagccctgaccagcggcgtgcacaccttcccggctgtcttgcagtcctcaggactctactccctcagcagcgtggtgaccgtccccagcagctccttgggcacccagacctacatctgcaacgtgaatcacaagcccagcaacaccaaggtggacaagaaagttgagcccaaatcttgtgacaaaactcacacatgcccaccgtgcccagcacctgagctcctggggggaccgtcagtgttcctcttccccccaaaacccaaggacaccctcatgatctcccggacccccgaggtcacatgcgtggtggtggacgtgagccacgaggaccctgaagtgaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtcagcaacaaagccctcccagcccccatcgagaaaaccattagcaaagccaaagggcagccccgagaaccacaggtttacaccctgcccccatccagagatgagctgaccaagaaccaggtcagcctgacctgcctcgtgaaaggcttctatcccagcgacatcgccgtggagtgggagagcaatgggcagccggagaacaactacaagaccacgcctcccgtgctggactccgacggctccttcttcctctacagcaagctcaccgtggataagagcaggtggcagcaggggaacgtgttctcatgctccgtgatgcacgaggctctgcacaaccactacacgcagaagagcctctccctgtctccggggaaatga

SEQ ID NO: 80 - DNA, 654 bp, synthetic construct
  source 1..654: mol_type = other DNA
cagtctgtgctgactcagcctgcctccgtgtctgggagccctggacagtcgatcaccatctcctgcgctggaaccagcagtgacgttggtggttattacgacgtctcctggtaccaacaacacccaggcaaagcccccaaactcatgatttattacgacagttaccggccctcaggggtttctaatcgcttctctggctccaagtctggcaacacggcctccctgacaatctctgggctccaggctgaggacgaggctgattattactgcagctcatatacaagctacagcactcgagttttcggcggagggaccaagctggagatcaaaagaactgtggctgccccatctgttttcatcttcccgccatctgatgagcagttgaaatctggaactgcctctgttgtgtgcctgctgaataacttctatcccagagaggccaaagtccagtggaaggtggataacgccctccaatctggtaactcccaggagagtgtcacagagcaggacagcaaggacagcacctacagcctcagcagcaccctgacgctgagcaaagcagactacgagaaacacaaagtgtacgcctgcgaagtcacccatcagggcctgagcagtcccgtcacaaagagcttcaacaggggagagtgttag

SEQ ID NO: 81 - protein, 117 aa, synthetic construct
EVQLVESGGGLVKPGGSLRLSCAASGFTFSNYNMNWVRQAPGKGLEWISSIYGSSRYIYYADSVKGRFTISRDNAKNSLYLQMNSLRAEDTAVYYCVRSGYGGMDVWGRGTLVTVSS

SEQ ID NO: 82 - protein, 117 aa, synthetic construct
EVQLVESGGGLVKPGGSLRLSCAASGFTFSNYNMNWVRQAPGKGLEWVSSIYGSSRYIYYADSVKGRFTISRDNAKNSLYLQMNSLRAEDTAVYYCVRSGYGGMDVWGRGTLVTVSS

SEQ ID NO: 83 - protein, 110 aa, synthetic construct
QSALTQPASVSGSPGQSITISCTGTSSDVGGYNGVSWYQQHPGKAPKLMIYYDSYRPSGVSNRFSGSKSGNTASLTISGLQAEDEADYYCSSGTDYSTRVFGQGTKLEIK

SEQ ID NO: 84 - protein, 117 aa, synthetic construct
EVQLVESGGGLVKPGGSLRLSCAASGFTFSNYNMNWVRQAPGKGLEWISSIYGSSRYIYYADSVKGRFTISRDNAKNSLYLQMNSLRAEDTAVYYCVRSSYGGMDVWGRGTLVTVSS

SEQ ID NO: 85 - protein, 117 aa, synthetic construct
EVQLVESGGGLVKPGGSLRLSCAASGFTFSNYNMNWVRQAPGKGLEWVSSIYGSSRYIYYADSVKGRFTISRDNAKNSLYLQMNSLRAEDTAVYYCVRSSYGGMDVWGRGTLVTVSS

SEQ ID NO: 86 - protein, 110 aa, synthetic construct
QSALTQPASVSGSPGQSITISCTGTSSDVGGSYNVAWYQQHPGKAPKLMIYYDSYRPSGVSNRFSGSKSGNTASLTISGLQAEDEADYYCSSYTGYSTRVFGGGTKLEIK

SEQ ID NO: 87 - protein, 117 aa, synthetic construct
EVQLVESGGGLVKPGGSLRLSCAASGFTFSNYNMNWVRQAPGKGLEWISNIYGSSRYIYYADSVKGRFTISRDNAKNSLYLQMNSLRAEDTAVYYCVRSYYGGMDVWGRGTLVTVSS

SEQ ID NO: 88 - protein, 117 aa, synthetic construct
EVQLVESGGGLVKPGGSLRLSCAASGFTFSNYNMNWVRQAPGKGLEWVSNIYGSSRYIYYADSVKGRFTISRDNAKNSLYLQMNSLRAEDTAVYYCVRSYYGGMDVWGRGTLVTVSS

SEQ ID NO: 89 - protein, 110 aa, synthetic construct
QSALTQPRSVSGSPGQSVTISCTGTSSDVGGYYSVSWYQQHPGKAPKLMIYYDSNRPSGVPDRFSGSKSGNTASLTISGLQAEDEADYYCSSYTGYSTRVFGQGTKLEIK

SEQ ID NO: 90 - protein, 123 aa, synthetic construct
EVQLVESGGGLVKPGGSLRLSCAASGFTFSNYYMNWVRQAPGKGLEWISYINGASRDISYADSVKGRFTISRDNAKNSLYLQMNSLRAEDTAVYYCVRSSYGYSNYGSGMDVWGRGTLVTVSS

SEQ ID NO: 91 - protein, 110 aa, synthetic construct
QSALTQPASVSGSPGQSITISCTGTSSDVGGSYNVAWYQQHPGKAPKLMIYNDANRPSGVSNRFSGSKSGNTASLTISGLQAEDEADYYCSSNTADSTRVFGGGTKLEIK

SEQ ID NO: 92 - protein, 119 aa, synthetic construct
EVQLVESGGGLVKPGGSLRLSCAASGFTFSNNYMNWVRQAPGKGLEWISNINGASRNIAYADSVKGRFTISRDNAKNSLYLQMNSLRAEDTAVYYCVRSSDYDGGMDVWGRGTLVTVSS

SEQ ID NO: 93 - protein, 110 aa, synthetic construct
QSALTQPASVSGSPGQSITISCTGTSSDVGGNSYVSWYQQHPGKAPKLMIYYDSYRPSGVSNRFSGSKSGNTASLTISGLQAEDEADYYCSSSTYGSTRVFGGGTKLEIK

SEQ ID NO: 94 - protein, 117 aa, synthetic construct
EVQLVESGGGLVKPGGSLRLSCAASGFTFSNYAMNWVRQAPGKGLEWISSIYGSSRYIYYADSVKGRFTISRDNAKNSLYLQMNSLRAEDTAVYYCVRSYNGGMDVWGRGTLVTVSS

SEQ ID NO: 95 - protein, 117 aa, synthetic construct
EVQLVESGGGLVKPGGSLRLSCAASGFTFSNYAMNWVRQAPGKGLEWVSSIYGSSRYIYYADSVKGRFTISRDNAKNSLYLQMNSLRAEDTAVYYCVRSYNGGMDVWGRGTLVTVSS

SEQ ID NO: 96 - protein, 110 aa, synthetic construct
QSALTQPRSVSGSPGQSVTISCTGTSSDVGGYYNVSWYQQHPGKAPKLMIYYDSYRPSGVPDRFSGSKSGNTASLTISGLQAEDEADYYCSSSTGYSTRVFGGGTKLEIK

SEQ ID NO: 97 - protein, 117 aa, synthetic construct
EVQLVESGGGLVKPGGSLRLSCAASGFTFSNYNMNWVRQAPGKGLEWISNIDGSSRYIYYADSVKGRFTISRDNAKNSLYLQMNSLRAEDTAVYYCVRSYYGGMDVWGRGTLVTVSS

SEQ ID NO: 98 - protein, 110 aa, synthetic construct
QSALTQPRSVSGSPGQSVTISCAGTSSDVGGYYNVSWYQQHPGKAPKLMIYYDSYRPSGVPDRFSGSKSGNTASLTISGLQAEDEADYYCSSSTGYSTRVFGGGTKLEIK

SEQ ID NO: 99 - protein, 122 aa, synthetic construct
EVQLVESGGGLVKPGGSLRLSCAASGFTFSNYAMNWVRQAPGKGLEWISSIYGSSRSISYADFVKGRFTISRDNAKNSLYLQMNSLRAEDTAVYYCVRSSYNYYYGNGMDVWGRGTLVTVSS

SEQ ID NO: 100 - protein, 110 aa, synthetic construct
QSALTQPASVSGSPGQSITISCTGTSSDVGGYGGVSWYQQHPGKAPKLMIYYDSYRPSGVSNRFSGSKSGNTASLTISGLQAEDEADYYCSSSTSYSTRVFGQGTKLEIK

SEQ ID NO: 101 - protein, 120 aa, synthetic construct
EVQLVESGGGLVKPGGSLRLSCAASGFTFSNDDMNWVRQAPGKGLEWISSIDGSSRSIYYADSVKGRFTISRDNAKNSLYLQMNSLRAEDTAVYYCVRSSGYSGGGMDVWGRGTLVTVSS

SEQ ID NO: 102 - protein, 110 aa, synthetic construct
QSALTQPASVSGSPGQSITISCAGTSSDVGGSYNVAWYQQHPGKAPKLMIYSDSYRPSGVSNRFSGSKSGNTASLTISGLQAEDEADYYCSSYTSNSARVFGGGTKLEIK

SEQ ID NO: 103 - protein, 117 aa, synthetic construct
EVQLVESGGGLVQPGGSLRLSCAASGFTFSNYAMNWVRQAPGKGLEWISSIYGSSRSIYYADSVKGRFTISRDNAKNSLYLQMNSLRAEDTAVYYCVRSNNAGMDVWGRGTLVTVSS

SEQ ID NO: 104 - protein, 117 aa, synthetic construct
EVQLVESGGGLVQPGGSLRLSCAASGFTFSNYAMNWVRQAPGKGLEWVSSIYGSSRSIYYADSVKGRFTISRDNAKNSLYLQMNSLRAEDTAVYYCVRSNNAGMDVWGRGTLVTVSS

SEQ ID NO: 105 - protein, 110 aa, synthetic construct
QSALTQPASVSGSPGQSITISCTGTSSDVGGYYDVSWYQQHPGKAPKLMIYYDSYRPSGVSNRFSGSKSGNTASLTISGLQAEDEADYYCSSYTSYSTRVFGQGTKLEIK

SEQ ID NO: 106 - protein, 250 aa, Homo sapiens [sequence omitted]

SEQ ID NO: 107 - protein, 145 aa, Homo sapiens
PYGAPAGPLIVPYNLPLPGGVVPRMLITILGTVKPNANRIALDFQRGNDVAFHFNPRFNENNRRVIVCNTKLDNNWGREERQSVFPFESGKPFKIQVLVEPDHFKVAVNDAHLLQYNHRVKKLNEISKLGISGDIDLTSASYTMI

SEQ ID NO: 108 - protein, 135 aa, Homo sapiens
MACGLVASNLNLKPGECLRVRGEVAPDAKSFVLNLGKDSNNLCLHFNPRFNAHGDANTIVCNSKDGGAWGTEQREAVFPFQPGSVAEVCITFDQANLTVKLPDGYEFKFPNRLNLEAINYMAADGDFKIKCVAFD

SEQ ID NO: 109 - protein, 136 aa, Homo sapiens
MSNVPHKSSLPEGIRPGTVLRIRGLVPPNASRFHVNLLCGEEQGSDAALHFNPRLDTSEVVFNSKEQGSWGREERGPGVPFQRGQPFEVLIIASDDGFKAVVGDAQYHHFRHRLPLARVRLVEVGGDVQLDSVRIF

SEQ ID NO: 110 - protein, 264 aa, Mus musculus
MADSFSLNDALAGSGNPNPQGYPGAWGNQPGAGGYPGAAYPGAYPGQAPPGAYPGQAPPGAYPGQAPPSAYPGPTAPGAYPGPTAPGAYPGQPAPGAFPGQPGAPGAYPQCSGGYPAAGPYGVPAGPLTVPYDLPLPGGVMPRMLITIMGTVKPNANRIVLDFRRGNDVAFHFNPRFNENNRRVIVCNTKQDNNWGKEERQSAFPFESGKPFKIQVLVEADHFKVAVNDAHLLQYNHRMKNLREISQLGISGDITLTSANHAMI